(19) United States
(12) Patent Application Publication
(10) Pub. No.: US 2023/0128259 A1
(43) Pub. Date: Apr. 27, 2023

(54) COMPOUND AND ORGANIC LIGHT-EMITTING DEVICE COMPRISING SAME

(71) Applicant: LG CHEM, LTD., Seoul (KR)

(72) Inventors: Miyeon HAN, Daejeon (KR); Sung Kil HONG, Daejeon (KR); Dong Uk HEO, Daejeon (KR); Jung Min YOON, Daejeon (KR); Heekyung YUN, Daejeon (KR)

(21) Appl. No.: 17/911,901

(22) PCT Filed: Apr. 29, 2021
(86) PCT No.: PCT/KR2021/005434

§ 371 Date: Sep. 15, 2022

(30) Foreign Application Priority Data
May 28, 2020 (KR) 10-2020-0064408

(51) Int. Cl. H10K 85/60; C07C 255/50; C09K 11/06; C07C 255/51; C07D 213/57; C07C 255/52; C07D 239/74; C07D 307/91; C07D 209/88; C07D 251/24; C07C 255/54; C07D 333/76

(52) U.S. Cl. CPC H01L 51/0054; C07C 255/50; C09K 11/06; C07C 255/51; C07D 213/57; H01L 51/0067; C07C 255/52; H01L 51/0058; C07D 239/74; H01L 51/0072; C07D 307/91; H01L 51/0073; C07D 209/88; C07D 251/24; C07C 255/54; H01L 51/0055; H01L 51/0056; C07D 333/76; H01L 51/0074; H01L 51/5072

(57) ABSTRACT

Provided is a compound of Chemical Formula 1:

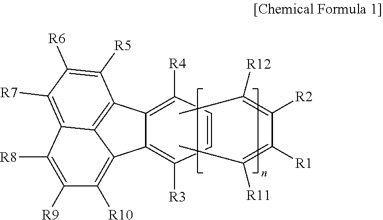

wherein n is 0 or 1, when n is 0, at least one of R1 to R10 is a group of Chemical Formula A, and when n is 1, at least one of R1 to R12 is a group of Chemical Formula A:

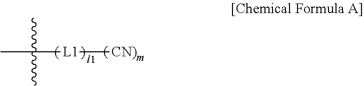

wherein L1 is a direct bond, or a substituted or unsubstituted arylene or heteroarylene group; l1 is an integer of 1 to 5; m is an integer of 1 to 3; when l1 is 2 or greater, the two or more L1s are the same as or different from each other, and an organic light emitting device including the same.

### CROSS-REFERENCE TO RELATED APPLICATION(S)

This application is a National Stage Application of International Application No. PCT/KR2021/005434 filed on Apr. 29, 2021, which claims priority to and the benefits of Korean Patent Application No. 10-2020-0064408, filed with the Korean Intellectual Property Office on May 28, 2020, the entire contents of which are incorporated herein by reference.

### TECHNICAL FIELD

The present specification relates to a compound, and an organic light emitting device including the same.

### BACKGROUND

An organic light emission phenomenon generally refers to a phenomenon converting electrical energy to light energy using an organic material. An organic light emitting device using an organic light emission phenomenon normally has a structure including an anode, a cathode, and an organic material layer therebetween. Herein, the organic material layer is often formed in a multilayer structure formed with different materials in order to increase efficiency and stability of the organic light emitting device, and for example, can be formed with a hole injection layer, a hole transfer layer, a light emitting layer, an electron transfer layer, an electron injection layer and the like. When a voltage is applied between the two electrodes in such an organic light emitting device structure, holes and electrons are injected to the organic material layer from the anode and the cathode, respectively, and when the injected holes and electrons meet, excitons are formed, and light emits when these excitons fall back to the ground state.

Development of new materials for such an organic light emitting device has been continuously required.

### BRIEF DESCRIPTION

### Technical Problem

The present specification is directed to providing a compound, and an organic light emitting device including the same.

### Technical Solution

One embodiment of the present specification provides a compound of Chemical Formula 1:

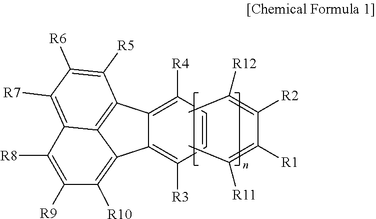

wherein in Chemical Formula 1:

n is 0 or 1;

when n is 0, at least one of R1 to R10 is a group of the following Chemical Formula A;

when n is 1, at least one of R1 to R12 is a group of the following Chemical Formula A; and

the rest of the groups among R1 to R12 other than the group of the following Chemical Formula A are the same as or different from each other, and each independently is hydrogen, deuterium, a halogen group, a cyano group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group, or adjacent groups bond to each other to form a substituted or unsubstituted ring;

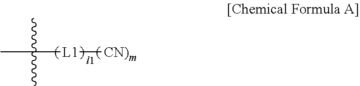

wherein in Chemical Formula A:

L1 is a direct bond, a substituted or unsubstituted arylene group, or a substituted or unsubstituted heteroarylene group;

l1 is an integer of 1 to 5;

m is an integer of 1 to 3;

when l1 is 2 or greater, the two or more L1s are the same as or different from each other;

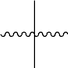

means a site directly bonding to at least one of R1 to R10 or at least one of R1 to R12 of Chemical Formula 1;

when n is 1 and R1 and R2 are the rest of the groups other than the group of Chemical Formula A, at least one of R1 and R2 is deuterium, a halogen group, a cyano group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group, or adjacent groups bond to each other to form a substituted or unsubstituted; and

when n is 0, R1 or R2 is the group of Chemical Formula A, l1 is 1, m is 1 and L1 is a phenylene group, R1 or R2 is a group of the following Chemical Formula A-1 or A-2:

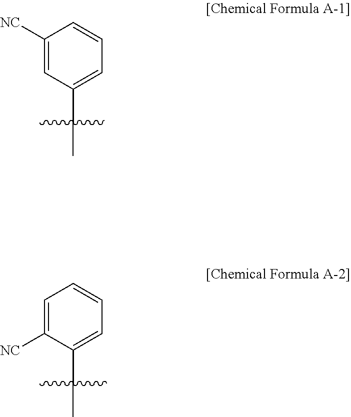 [SMILES: *c1cccc(C#N)c1 ; *c1ccccc1C#N]

wherein in Chemical Formulae A-1 and A-2:

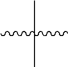

means a site directly bonding to R1 or R2 of Chemical Formula 1.

Another embodiment of the present specification provides an organic light emitting device including a first electrode; a second electrode provided opposite to the first electrode; and one or more organic material layers provided between the first electrode and the second electrode, wherein one or more layers of the organic material layers include the compound.

### Advantageous Effects

A compound according to one embodiment of the present specification can be used as a material of an organic material layer of an organic light emitting device, and by using the same, efficiency can be enhanced, low driving voltage can be obtained and/or lifetime properties can be enhanced in the organic light emitting device.

### DESCRIPTION OF DRAWINGS

FIG. 1 and FIG. 2 illustrate examples of an organic light emitting device according to one embodiment of the present specification.

### REFERENCE NUMERALS

101: Substrate
102: First Electrode
111: Organic Material Layer
110: Second Electrode
103: Hole Injection Layer
104: First Hole Transfer Layer
105: Second Hole Transfer Layer
106: Light Emitting Layer
107: Electron Injection and Transfer Layer

### DETAILED DESCRIPTION

Hereinafter, the present specification will be described in more detail.

One embodiment of the present specification provides a compound of Chemical Formula 1.

Chemical Formula 1 according to one embodiment of the present specification is a compound in which a fluoranthene or benzofluoranthene core includes a cyano group, and, by being included in an organic material layer of an organic light emitting device, is capable of enhancing efficiency, obtaining a low driving voltage, enhancing lifetime properties, and the like. Specifically, the fluoranthene or benzofluoranthene structure that is the core structure of Chemical Formula 1 has effects of preventing crystallization occurring when forming a film through steric hinderance, and being very stable even at a high deposition temperature by maintaining high thermal stability. In addition, since the cyano group is an electron withdrawing group and is thereby capable of increasing molecular polarity (dipole moment), electron mobility can be smoothly controlled when manufacturing an organic light emitting device including the compound of Chemical Formula 1, and as a result, efficiency and lifetime of the organic light emitting device including the compound of Chemical Formula 1 can be enhanced.

In addition, when n is 0, R1 or R2 is a group of Chemical Formula A, l1 is 1, m is 1 and L1 is a phenylene group in Chemical Formula 1, Chemical Formula A includes a structure of Chemical Formula A-1 (the cyano group is linked to a meta position of the phenylene group) or A-2 (the cyano group is linked to an ortho position of the phenylene group), which decreases structural flatness compared to a compound including a structure having a cyano group linked to a para position of a phenylene group in R1 or R2 due to steric hindrance, and provides flexibility in bonding allowing to readily laminate the compound, and as a result, efficiency and voltage properties of an organic light emitting device including the same are improved since electrons are readily transferred.

Examples of substituents in the present specification are described below, however, the substituents are not limited thereto.

In the present specification,

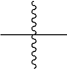

means a linking site.

The term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents can be the same as or different from each other.

In the present specification, a term “substituted or unsubstituted” means being substituted with one or more substituents selected from the group consisting of deuterium, a halogen group, a cyano group, an alkyl group, a cycloalkyl group, an alkoxy group, an alkenyl group, a haloalkyl group, a silyl group, a boron group, an amine group, an aryl group, and a heteroaryl group, or being substituted with a substituent linking two or more substituents among the substituents illustrated above, or having no substituents.

In the present specification, linking two or more substituents refers to linking hydrogen of any one substituent to another substituent. For example, linking two or more substituents can include a phenyl group and a naphthyl group being linked to become a substituent of

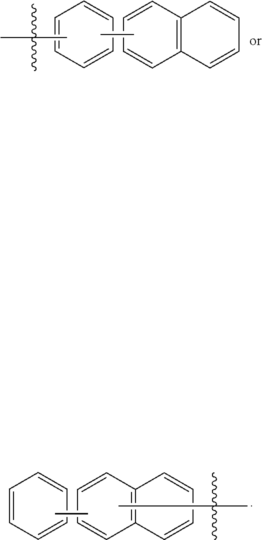

In addition, linking three substituents includes not only continuously linking (substituent 1)-(substituent 2)-(substituent 3), but also linking (substituent 2) and (substituent 3) to (substituent 1). For example, a phenyl group, a naphthyl group and an isopropyl group can be linked to become a substituent of

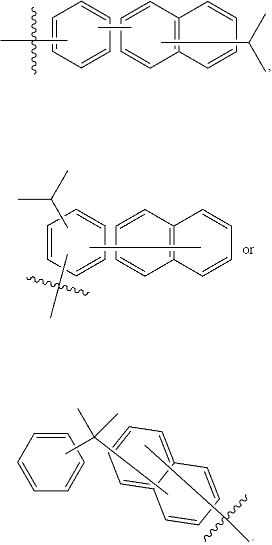

The same rule described above applies to cases of linking four or more substituents.

In the present specification, examples of the halogen group can include fluorine, chlorine, bromine or iodine.

In the present specification, the alkyl group can be linear or branched, and although not particularly limited thereto, the number of carbon atoms is preferably from 1 to 30. Specific examples thereof can include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl and the like, but are not limited thereto.

In the present specification, the cycloalkyl group is not particularly limited, but preferably has 3 to 30 carbon atoms. Specific examples thereof can include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, an adamantyl group and the like, but are not limited thereto.

In the present specification, the alkoxy group can be linear, branched or cyclic. The number of carbon atoms of the alkoxy group is not particularly limited, but is preferably from 1 to 30. Specific examples thereof can include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the alkenyl group can be linear or branched, and although not particularly limited thereto, the number of carbon atoms is preferably from 2 to 30. Specific examples thereof can include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group and the like, but are not limited thereto.

In the present specification, the haloalkyl group means, in the definition of the alkyl group, hydrogen of the alkyl group being substituted with at least one halogen group.

In the present specification, the aryl group is not particularly limited, but preferably has 6 to 30 carbon atoms, and the aryl group can be monocyclic or polycyclic.

When the aryl group is a monocyclic aryl group, the number of carbon atoms is not particularly limited, but is preferably from 6 to 30. Specific examples of the monocyclic aryl group can include a phenyl group, a biphenyl group, a terphenyl group and the like, but are not limited thereto.

When the aryl group is a polycyclic aryl group, the number of carbon atoms is not particularly limited, but is preferably from 10 to 30. Specific examples of the polycyclic aryl group can include a naphthyl group, an anthracene group, a phenanthrene group, a triphenylene group, a pyrene group, a phenalene group, a perylene group, a chrysene group, a fluorene group and the like, but are not limited thereto.

In the present specification, the fluorene group can be substituted, and adjacent groups can bond to each other to form a ring.

When the fluorene group is substituted,

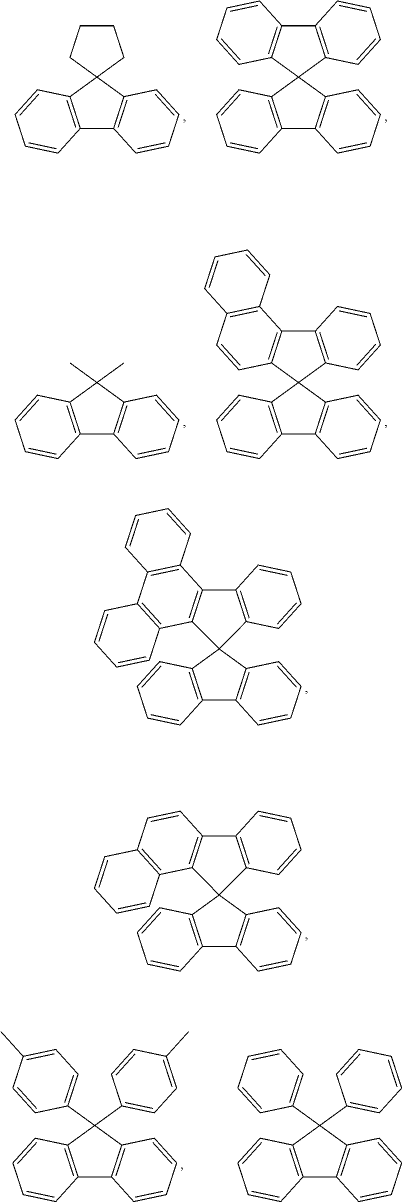

and the like can be included, however, the structure is not limited thereto.

In the present specification, an “adjacent” group can mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring can be interpreted as groups “adjacent” to each other.

In the present specification, the heteroaryl group is a group including one or more atoms that are not carbon, that is, heteroatoms, and specifically, the heteroatom can include one or more atoms selected from the group consisting of O, N, Se, S and the like. The number of carbon atoms is not particularly limited, but is preferably from 2 to 30, and the heteroaryl group can be monocyclic or polycyclic. Examples of the heterocyclic group can include a thiophene group, a furan group, a pyrrole group, an imidazole group, a thiazole group, an oxazole group, an oxadiazole group, a pyridine group, a bipyridine group, a pyrimidine group, a triazine group, a triazole group, an acridine group, a pyridazine group, a pyrazine group, a quinoline group, a quinazoline group, a quinoxaline group, a phthalazine group, a pyridopyrimidine group, a pyridopyrazine group, a pyrazinopyrazine group, an isoquinoline group, an indole group, a carbazole group, a benzoxazole group, a benzimidazole group, a benzothiazole group, a benzocarbazole group, a benzothiophene group, a dibenzothiophene group, a benzofuran group, a phenanthridine group, a phenanthroline group, an isoxazole group, a thiadiazole group, a dibenzofuran group, dibenzosilole group, a phenoxanthine group, a phenoxazine group, a phenothiazine group, a dihydroindenocarbazole group, a spirofluorenexanthene group, a spirofluorenethioxanthene group and the like, but are not limited thereto.

In the present specification, the silyl group can be an alkylsilyl group, an arylsilyl group, a heteroarylsilyl group or the like. As the alkyl group in the alkylsilyl group, the examples of the alkyl group described above can be applied, and as the aryl group in the arylsilyl group, the examples of the aryl group described above can be applied, and as the heteroaryl group in the heteroarylsilyl group, the examples of the heteroaryl group can be applied.

In the present specification, the boron group can be —BR₁₀₀R₁₀₁. R₁₀₀ and R₁₀₁ are the same as or different from each other, and can be each independently selected from the group consisting of hydrogen, deuterium, halogen, a nitrile group, a substituted or unsubstituted monocyclic or polycyclic cycloalkyl group having 3 to 30 carbon atoms, a substituted or unsubstituted linear or branched alkyl group having 1 to 30 carbon atoms, a substituted or unsubstituted monocyclic or polycyclic aryl group having 6 to 30 carbon atoms, and a substituted or unsubstituted monocyclic or polycyclic heteroaryl group having 2 to 30 carbon atoms. Specific examples of the boron group can include a trimethylboron group, a triethylboron group, a t-butyldimethylboron group, a triphenylboron group, a phenylboron group and the like, but are not limited thereto.

In the present specification, the amine group can be selected from the group consisting of —NH₂, an alkylamine group, an N-alkylarylamine group, an arylamine group, an N-arylheteroarylamine group, an N-alkylheteroarylamine group and a heteroarylamine group, and although not particularly limited thereto, the number of carbon atoms is preferably from 1 to 30. Specific examples of the amine group can include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, a ditolylamine group, an N-phenyltolylamine group, a triphenylamine group, an N-phenylbiphenylamine group, an N-phenylnaphthylamine group, an N-biphenylnaphthylamine group, an N-naphthylfluorenylamine group, an N-phenylphenanthrenylamine group, an N-biphenylphenanthrenylamine group, an N-phenylfluorenylamine group, an N-phenylterphenylamine group, an N-phenanthrenylfluorenylamine group, an N-biphenylfluorenylamine group and the like, but are not limited thereto.

In the present specification, the N-alkylarylamine group means an amine group in which N of the amine group is substituted with an alkyl group and an aryl group. The alkyl group and the aryl group in the N-alkylarylamine group are the same as the examples of the alkyl group and the aryl group described above.

In the present specification, the N-arylheteroarylamine group means an amine group in which N of the amine group is substituted with an aryl group and a heteroaryl group. The aryl group and the heteroaryl group in the N-arylheteroarylamine group are the same as the examples of the aryl group and the heteroaryl group described above.

In the present specification, the N-alkylheteroarylamine group means an amine group in which N of the amine group is substituted with an alkyl group and a heteroaryl group. The alkyl group and the heteroaryl group in the N-alkylheteroarylamine group are the same as the examples of the alkyl group and the heteroaryl group described above.

In the present specification, examples of the arylamine group include a substituted or unsubstituted monoarylamine group, or a substituted or unsubstituted diarylamine group. The arylamine group including two or more aryl groups can include monocyclic aryl groups, polycyclic aryl groups, or both monocyclic aryl groups and polycyclic aryl groups. For example, the aryl group in the arylamine group can be selected from among the examples of the aryl group described above.

In the present specification, examples of the heteroarylamine group include a substituted or unsubstituted monoheteroarylamine group, or a substituted or unsubstituted diheteroarylamine group. The heteroarylamine group including two or more heteroaryl groups can include monocyclic heteroaryl groups, polycyclic heteroaryl groups, or both monocyclic heteroaryl groups and polycyclic heteroaryl groups. For example, the heteroaryl group in the heteroarylamine group can be selected from among the examples of the heteroaryl group described above. For example, the heteroaryl group in the heteroarylamine group can be selected from among the examples of the heteroaryl group described above.

In the present specification, the alkyl group in the alkylthioxy group and the alkylsulfoxy group is the same as the examples of the alkyl group described above. Specific examples of the alkylthioxy group can include a methylthioxy group, an ethylthioxy group, a tert-butylthioxy group, a hexylthioxy group, an octylthioxy group and the like, and specific examples of the alkylsulfoxy group can include a methylsulfoxy group, an ethylsulfoxy group, a propylsulfoxy group, a butylsulfoxy group and the like, however, the alkylthioxy group and the alkylsulfoxy group are not limited thereto.

In the present specification, the phosphine oxide group can specifically be an alkylphosphine oxide group, an arylphosphine oxide group or the like, and more specifically be a diphenylphosphine oxide group, a dinaphthylphosphine oxide group or the like, however, the phosphine oxide group is not limited thereto.

In the present specification, the aryl group in the aryloxy group, the arylthioxy group, the arylsulfoxy group and the arylphosphine group is the same as the examples of the aryl group described above. Specific examples of the aryloxy group can include a phenoxy group, a p-tolyloxy group, an m-tolyloxy group, a 3,5-dimethyl-phenoxy group, a 2,4,6-trimethylphenoxy group, a p-tert-butylphenoxy group, a 3-biphenyloxy group, a 4-biphenyloxy group, a 1-naphthyloxy group, a 2-naphthyloxy group, a 4-methyl-1-naphthyloxy group, a 5-methyl-2-naphthyloxy group, a 1-anthryloxy group, a 2-anthryloxy group, a 9-anthryloxy group, a 1-phenanthryloxy group, a 3-phenanthryloxy group, a 9-phenanthryloxy group and the like, examples of the arylthioxy group can include a phenylthioxy group, a 2-methylphenylthioxy group, a 4-tert-butylphenylthioxy group and the like, and examples of the arylsulfoxy group can include a benzenesulfoxy group, a p-toluenesulfoxy group and the like, however, the aryloxy group, the arylthioxy group and the arylsulfoxy group are not limited thereto.

In the present specification, the meaning of “adjacent two of substituents bonding to each other to form a ring” means bonding to adjacent groups to form a substituted or unsubstituted hydrocarbon ring; or a substituted or unsubstituted heteroring.

In the present specification, the “ring” in the substituted or unsubstituted ring formed by bonding to each other means a substituted or unsubstituted hydrocarbon ring; or a substituted or unsubstituted heteroring.

In the present specification, the hydrocarbon ring can be an aromatic hydrocarbon ring, an aliphatic hydrocarbon ring, or a fused ring of aromatic hydrocarbon and aliphatic hydrocarbon, and can be selected from among the examples of the cycloalkyl group or the aryl group except for those that are not monovalent.

In the present specification, the heteroring means a ring including one or more atoms that are that carbon, that is, heteroatoms, and specifically, the heteroatom can include one or more atoms selected form the group consisting of O, N, Se, S and the like. The heteroring can be monocyclic or polycyclic, and can be aromatic, aliphatic, or a fused ring of aromatic and aliphatic. The aromatic heteroring can be selected from among the examples of the heteroaryl group except for those that are not monovalent.

In the present specification, the aliphatic heteroring means an aliphatic ring including one or more of heteroatoms. Examples of the aliphatic heteroring can include oxirane, tetrahydrofuran, 1,4-dioxane, pyrrolidine, piperidine, morpholine, oxepane, azokane, thiokane and the like, but are not limited thereto.

In the present specification, the arylene group means the aryl group having two bonding sites, that is, a divalent group. The descriptions on the aryl group provided above can be applied thereto except for those that are each a divalent group.

In the present specification, the heteroarylene group means the heteroaryl group having two bonding sites, that is, a divalent group. The descriptions on the heteroaryl group provided above can be applied thereto except for those that are each a divalent group.

Hereinafter, the compound of Chemical Formula 1 will be described in detail.

According to one embodiment of the present specification, n is 0.

According to one embodiment of the present specification, n is 1.

According to one embodiment of the present specification, Chemical Formula 1 is the following Chemical Formula 1-1 or 1-2:

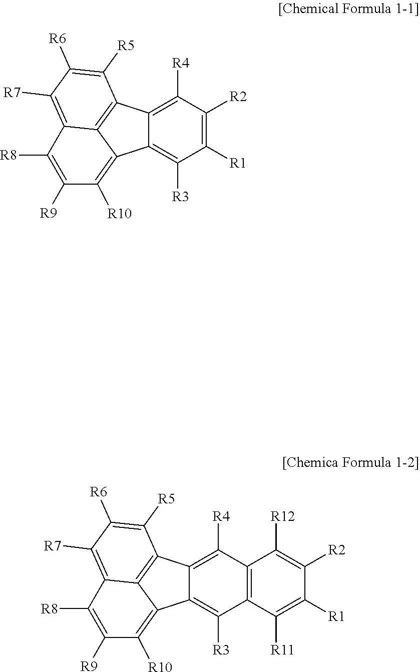

wherein in Chemical Formulae 1-1 and 1-2:

R1 to R12 have the same definitions as in Chemical Formula 1.

According to one embodiment of the present specification, Chemical Formula 1 is the following Chemical Formula 1-3 or 1-4:

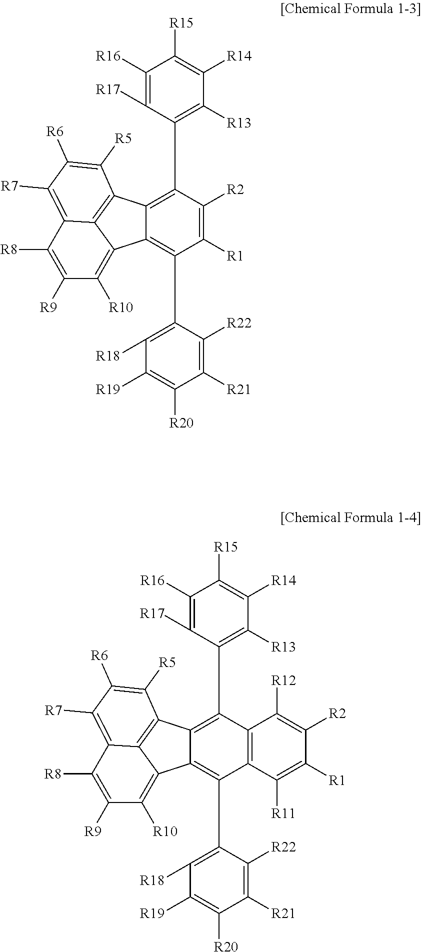

wherein in Chemical Formulae 1-3 and 1-4:

R1, R2 and R5 to R12 have the same definitions as in Chemical Formula 1; and

R13 to R22 are the same as or different from each other, and each independently is hydrogen, deuterium, a halogen group, a cyano group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group, or bond to adjacent groups to form a substituted or unsubstituted ring.

According to one embodiment of the present specification, Chemical Formula A is any one of the following Chemical Formulae A-3 to A-5:

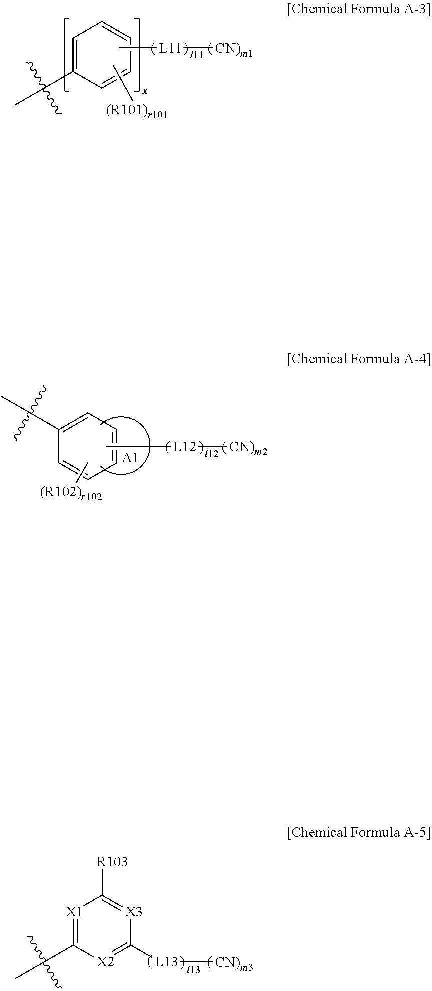

wherein in Chemical Formulae A-3 to A-5:

x is 0 or 1;

L11 to L13 are the same as or different from each other, and each independently is a direct bond, a substituted or unsubstituted arylene group, or a substituted or unsubstituted heteroarylene group;

r101 is an integer of 1 to 4;

r102 is an integer of 1 to 3;

R101 and R102 are the same as or different from each other, and each independently is hydrogen, deuterium, a halogen group, a cyano group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group, or bond to adjacent groups to form a substituted or unsubstituted ring;

when r101 is 2 or greater, the two or more R101s are the same as or different from each other;

when r102 is 2 or greater, the two or more R102s are the same as or different from each other;

A1 is a substituted or unsubstituted hydrocarbon ring or a substituted or unsubstituted heteroring;

at least one of X1 to X3 is N, and the rest are CR;

R and R103 are the same as or different from each other, and each independently is hydrogen, deuterium, a halogen group, a cyano group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group, or adjacent two or more groups among R, R103 and L13 bond to each other to form a substituted or unsubstituted ring;

m1 to m3 are each an integer of 1 to 3;

l11 is an integer of 1 to 4;

l12 is an integer of 1 to 4;

l13 is an integer of 1 to 4;

when l11 is 2 or greater, the two or more L11s are the same as or different from each other;

when l12 is 2 or greater, the two or more L12s are the same as or different from each other; and

when l13 is 2 or greater, the two or more L13s are the same as or different from each other.

According to one embodiment of the present specification, l1 is 1.

According to one embodiment of the present specification, l1 is 2.

According to one embodiment of the present specification, l1 is 3.

According to one embodiment of the present specification, l1 is 4.

According to one embodiment of the present specification, l1 is 5.

In the present specification, when l1 is 2 or greater in Chemical Formula A, the two or more L2s are the same as or different from each other, and each L2 is connected in series. For example, when l1 is 3 and the L1s are each a phenylene group, a naphthylene group and a phenylene group, they can be connected as follows. However, the structure is not limited thereto, and the order or the connection position of each L1 can vary.

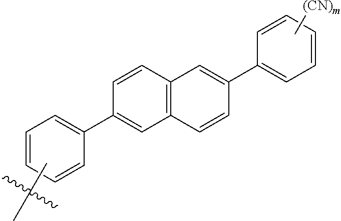

In addition, CN of Chemical Formula A means, when l1 is 3 as an example, bonding to the substituent positioned at the third end in the structure illustrated above, that is, the phenylene group.

According to one embodiment of the present specification, the rest of the groups among R1 to R12 other than the group of Chemical Formula A are the same as or different from each other, and each independently is hydrogen, a substituted or unsubstituted alkyl group, a substituted or unsubstituted aryl group, or a substituted or unsubstituted heteroaryl group, or adjacent groups bond to each other to form a substituted or unsubstituted ring.

According to one embodiment of the present specification, the rest of the groups among R1 to R12 other than the group of Chemical Formula A are the same as or different from each other, and each independently is hydrogen, a substituted or unsubstituted linear or branched alkyl group having 1 to 30 carbon atoms, a substituted or unsubstituted monocyclic or polycyclic aryl group having 6 to 30 carbon atoms, or a substituted or unsubstituted monocyclic or polycyclic heteroaryl group having 2 to 30 carbon atoms, or adjacent groups bond to each other to form a substituted or unsubstituted monocyclic or polycyclic aromatic hydrocarbon ring having 6 to 30 carbon atoms.

According to one embodiment of the present specification, the rest of the groups among R1 to R12 other than the group of Chemical Formula A are the same as or different from each other, and each independently is hydrogen, a substituted or unsubstituted linear or branched alkyl group having 1 to 20 carbon atoms, a substituted or unsubstituted monocyclic or polycyclic aryl group having 6 to 20 carbon atoms, or a substituted or unsubstituted monocyclic or polycyclic heteroaryl group having 2 to 20 carbon atoms, or adjacent groups bond to each other to form a substituted or unsubstituted monocyclic or polycyclic aromatic hydrocarbon ring having 6 to 20 carbon atoms.

According to one embodiment of the present specification, the rest of the groups among R1 to R12 other than the group of Chemical Formula A are the same as or different from each other, and each independently hydrogen; an alkyl group; an aryl group that is unsubstituted or substituted with an alkyl group, an alkoxy group, a haloalkyl group, an aryl group, or a heteroaryl group unsubstituted or substituted with an aryl group; or a heteroaryl group that is unsubstituted or substituted with an aryl group, or adjacent groups bond to each other to form a ring.

According to one embodiment of the present specification, the rest of the groups among R1 to R12 other than the group of Chemical Formula A are the same as or different from each other, and each independently is hydrogen; a linear or branched alkyl group having 1 to 30 carbon atoms; a monocyclic or polycyclic aryl group having 6 to 30 carbon atoms that is unsubstituted or substituted with a linear or branched alkyl group having 1 to 30 carbon atoms, a linear or branched alkoxy group having 1 to 30 carbon atoms, a monocyclic or polycyclic aryl group having 6 to 30 carbon atoms, or a monocyclic or polycyclic heteroaryl group having 2 to 30 carbon atoms that is unsubstituted or substituted with a monocyclic or polycyclic aryl group having 6 to 30 carbon atoms; or a monocyclic or polycyclic heteroaryl group having 2 to 30 carbon atoms that is unsubstituted or substituted with a monocyclic or polycyclic aryl group having 6 to 30 carbon atoms, or adjacent groups bond to each other to form a monocyclic or polycyclic aromatic hydrocarbon ring having 6 to 30 carbon atoms.

According to one embodiment of the present specification, the rest of the groups among R1 to R12 other than the group of Chemical Formula A are the same as or different from each other, and each independently is hydrogen; a linear or branched alkyl group having 1 to 20 carbon atoms; a monocyclic or polycyclic aryl group having 6 to 20 carbon atoms that is unsubstituted or substituted with a linear or branched alkyl group having 1 to 20 carbon atoms, a linear or branched alkoxy group having 1 to 20 carbon atoms, a monocyclic or polycyclic aryl group having 6 to 20 carbon atoms, or a monocyclic or polycyclic heteroaryl group having 2 to 20 carbon atoms that is unsubstituted or substituted with a monocyclic or polycyclic aryl group having 6 to 20 carbon atoms; or a monocyclic or polycyclic heteroaryl group having 2 to 20 carbon atoms that is unsubstituted or substituted with a monocyclic or polycyclic aryl group having 6 to 20 carbon atoms, or adjacent groups bond to each other to form a monocyclic or polycyclic aromatic hydrocarbon ring having 6 to 20 carbon atoms.

According to one embodiment of the present specification, the rest of the groups among R1 to R12 other than the group of Chemical Formula A are the same as or different from each other, and each independently is hydrogen; an ethyl group; a tert-butyl group; an n-pentyl group; a phenyl group that is unsubstituted or substituted with a tert-butyl group, a methoxy group, a trifluoromethyl group, a phenyl group, a naphthyl group, a pyridine group, a carbazole group, or a carbazole group substituted with a phenyl group; a biphenyl group; a naphthyl group; a phenalene group; a fluoranthene group; a triphenylene group; a pyridine group; or a triazine group that is unsubstituted or substituted with a phenyl group, and adjacent groups bond to each other to form a benzene ring.

According to one embodiment of the present specification, the rest of the groups among R1 to R10 other than the group of Chemical Formula A are the same as or different from each other, and each independently is hydrogen, a substituted or unsubstituted alkyl group, a substituted or unsubstituted aryl group, or a substituted or unsubstituted heteroaryl group, or adjacent groups bond to each other to form a substituted or unsubstituted ring.

According to one embodiment of the present specification, the rest of the groups among R1 to R10 other than the group of Chemical Formula A are the same as or different from each other, and each independently is hydrogen, a substituted or unsubstituted linear or branched alkyl group having 1 to 30 carbon atoms, a substituted or unsubstituted monocyclic or polycyclic aryl group having 6 to 30 carbon atoms, or a substituted or unsubstituted monocyclic or polycyclic heteroaryl group having 2 to 30 carbon atoms, or adjacent groups bond to each other to form a substituted or unsubstituted monocyclic or polycyclic aromatic hydrocarbon ring having 6 to 30 carbon atoms.

According to one embodiment of the present specification, the rest of the groups among R1 to R10 other than the group of Chemical Formula A are the same as or different from each other, and each independently is hydrogen, a substituted or unsubstituted linear or branched alkyl group having 1 to 20 carbon atoms, a substituted or unsubstituted monocyclic or polycyclic aryl group having 6 to 20 carbon atoms, or a substituted or unsubstituted monocyclic or polycyclic heteroaryl group having 2 to 20 carbon atoms, or adjacent groups bond to each other to form a substituted or unsubstituted monocyclic or polycyclic aromatic hydrocarbon ring having 6 to 20 carbon atoms.

According to one embodiment of the present specification, the rest of the groups among R1 to R10 other than the group of Chemical Formula A are the same as or different from each other, and each independently is hydrogen; an alkyl group; an aryl group that is unsubstituted or substituted with an alkyl group, an alkoxy group, a haloalkyl group, an aryl group, or a heteroaryl group that is unsubstituted or substituted with an aryl group; or a heteroaryl group that is unsubstituted or substituted with an aryl group, or adjacent groups bond to each other to form a ring.

According to one embodiment of the present specification, the rest of the groups among R1 to R10 other than the group of Chemical Formula A are the same as or different from each other, and each independently is hydrogen; a linear or branched alkyl group having 1 to 30 carbon atoms; a monocyclic or polycyclic aryl group having 6 to 30 carbon atoms that is unsubstituted or substituted with a linear or branched alkyl group having 1 to 30 carbon atoms, a linear or branched alkoxy group having 1 to 30 carbon atoms, a monocyclic or polycyclic aryl group having 6 to 30 carbon atoms, or a monocyclic or polycyclic heteroaryl group having 2 to 30 carbon atoms that is unsubstituted or substituted with a monocyclic or polycyclic aryl group having 6 to 30 carbon atoms; or a monocyclic or polycyclic heteroaryl group having 2 to 30 carbon atoms that is unsubstituted or substituted with a monocyclic or polycyclic aryl group having 6 to 30 carbon atoms, or adjacent groups bond to each other to form a monocyclic or polycyclic aromatic hydrocarbon ring having 6 to 30 carbon atoms.

According to one embodiment of the present specification, the rest of the groups among R1 to R10 other than the group of Chemical Formula A are the same as or different from each other, and each independently is hydrogen; a linear or branched alkyl group having 1 to 20 carbon atoms; a monocyclic or polycyclic aryl group having 6 to 20 carbon atoms that is unsubstituted or substituted with a linear or branched alkyl group having 1 to 20 carbon atoms, a linear or branched alkoxy group having 1 to 20 carbon atoms, a monocyclic or polycyclic aryl group having 6 to 20 carbon atoms, or a monocyclic or polycyclic heteroaryl group having 2 to 20 carbon atoms that is unsubstituted or substituted with a monocyclic or polycyclic aryl group having 6 to 20 carbon atoms; or a monocyclic or polycyclic heteroaryl group having 2 to 20 carbon atoms that is unsubstituted or substituted with a monocyclic or polycyclic aryl group having 6 to 20 carbon atoms, or adjacent groups bond to each other to form a monocyclic or polycyclic aromatic hydrocarbon ring having 6 to 20 carbon atoms.

According to one embodiment of the present specification, the rest of the groups among R1 to R10 other than the group of Chemical Formula A are the same as or different from each other, and each independently is hydrogen; an ethyl group; a tert-butyl group; an n-pentyl group; a phenyl group that is unsubstituted or substituted with a tert-butyl group, a methoxy group, a trifluoromethyl group, a phenyl group, a naphthyl group, a pyridine group, a carbazole group, or a carbazole group substituted with a phenyl group; a biphenyl group; a naphthyl group; a phenalene group; a fluoranthene group; a triphenylene group; a pyridine group; or a triazine group that is unsubstituted or substituted with a phenyl group, and adjacent groups bond to each other to form a benzene ring.

According to one embodiment of the present specification, L1 is a direct bond, a substituted or unsubstituted monocyclic or polycyclic arylene group having 6 to 30 carbon atoms, or a substituted or unsubstituted monocyclic or polycyclic heteroarylene group having 2 to 30 carbon atoms.

According to one embodiment of the present specification, L1 is a direct bond, a substituted or unsubstituted monocyclic or polycyclic arylene group having 6 to 20 carbon atoms, or a substituted or unsubstituted monocyclic or polycyclic heteroarylene group having 2 to 20 carbon atoms.

According to one embodiment of the present specification, L1 is a direct bond; an arylene group that is unsubstituted or substituted with an alkyl group or an aryl group; or a heteroarylene group that is unsubstituted or substituted with an aryl group that is unsubstituted or substituted with a cyano group.

According to one embodiment of the present specification, L1 is a direct bond; an arylene group having 6 to 30 carbon atoms that is unsubstituted or substituted with a linear or branched alkyl group having 1 to 30 carbon atoms or a monocyclic or polycyclic aryl group having 6 to 30 carbon atoms; or a monocyclic or polycyclic heteroarylene group having 2 to 30 carbon atoms that is unsubstituted or substituted with a monocyclic or polycyclic aryl group having 6 to 30 carbon atoms that is unsubstituted or substituted with a cyano group.

According to one embodiment of the present specification, L1 is a direct bond; an arylene group having 6 to 20 carbon atoms that is unsubstituted or substituted with a linear or branched alkyl group having 1 to 20 carbon atoms or a monocyclic or polycyclic aryl group having 6 to 20 carbon atoms; or a monocyclic or polycyclic heteroarylene group having 2 to 20 carbon atoms that is unsubstituted or substituted with a monocyclic or polycyclic aryl group having 6 to 20 carbon atoms that is unsubstituted or substituted with a cyano group.

According to one embodiment of the present specification, L1 is a direct bond; a phenylene group; a biphenylylene group; a terphenylylene group; a naphthylene group; a divalent phenanthrene group; a divalent triphenylene group; a divalent fluoranthene group; a divalent fluorene group substituted with a methyl group or a phenyl group; a divalent pyridine group that is unsubstituted or substituted with a phenyl group; a divalent pyrimidine group that is unsubstituted or substituted with a phenyl group that is unsubstituted or substituted with a cyano group; a divalent pyridazine group; a divalent triazine group that is unsubstituted or substituted with a phenyl group; a divalent carbazole group that is unsubstituted or substituted with a phenyl group; a divalent quinazoline group; a divalent dibenzofuran group; or a divalent dibenzothiophene group.

According to one embodiment of the present specification, the rest of the groups among R1 to R12 other than the group of Chemical Formula A are the same as or different from each other, and each independently is hydrogen; a linear or branched alkyl group having 1 to 30 carbon atoms; an aryl group having 6 to 30 carbon atoms that is unsubstituted or substituted with a linear or branched alkyl group having 1 to 30 carbon atoms, a linear or branched alkoxy group having 1 to 30 carbon atoms, a linear or branched haloalkyl group having 1 to 30 carbon atoms, a monocyclic or polycyclic aryl group having 6 to 30 carbon atoms, or a monocyclic or polycyclic heteroaryl group having 2 to 30 carbon atoms that is unsubstituted or substituted with a monocyclic or polycyclic aryl group having 6 to 30 carbon atoms; or a monocyclic or polycyclic heteroaryl group having 2 to 30 carbon atoms that is unsubstituted or substituted with a monocyclic or polycyclic aryl group having 6 to 30 carbon atoms, or adjacent groups bond to each other to form a monocyclic or polycyclic aromatic hydrocarbon ring having 6 to 30 carbon atoms; and L1 is a direct bond; an arylene group having 6 to 30 carbon atoms that is unsubstituted or substituted with a linear or branched alkyl group having 1 to 30 carbon atoms or a monocyclic or polycyclic aryl group having 6 to 30 carbon atoms; or a monocyclic or polycyclic heteroarylene group having 2 to 30 carbon atoms that is unsubstituted or substituted with a monocyclic or polycyclic aryl group having 6 to 30 carbon atoms that is unsubstituted or substituted with a cyano group.

According to one embodiment of the present specification, A1 is a substituted or unsubstituted monocyclic or polycyclic aromatic hydrocarbon ring having 6 to 30 carbon atoms, or a substituted or unsubstituted monocyclic or polycyclic heteroring having 2 to 30 carbon atoms.

According to one embodiment of the present specification, A1 is a substituted or unsubstituted monocyclic or polycyclic aromatic hydrocarbon ring having 6 to 20 carbon atoms, or a substituted or unsubstituted monocyclic or polycyclic heteroring having 2 to 20 carbon atoms.

According to one embodiment of the present specification, A1 is a monocyclic or polycyclic aromatic hydrocarbon ring having 6 to 30 carbon atoms that is unsubstituted or substituted with a linear or branched alkyl group having 1 to 30 carbon atoms or a monocyclic or polycyclic aryl group having 6 to 30 carbon atoms; or a monocyclic or polycyclic heteroring having 2 to 30 carbon atoms that is unsubstituted or substituted with a monocyclic or polycyclic aryl group having 6 to 30 carbon atoms.

According to one embodiment of the present specification, A1 is a monocyclic or polycyclic aromatic hydrocarbon ring having 6 to 20 carbon atoms that is unsubstituted or substituted with a linear or branched alkyl group having 1 to 20 carbon atoms or a monocyclic or polycyclic aryl group having 6 to 20 carbon atoms; or a monocyclic or polycyclic heteroring having 2 to 20 carbon atoms that is unsubstituted or substituted with a monocyclic or polycyclic aryl group having 6 to 20 carbon atoms.

According to one embodiment of the present specification, A1 is benzene; naphthalene; phenanthrene; indene that is unsubstituted or substituted with a methyl group or a phenyl group; benzimidazole that is unsubstituted or substituted with a phenyl group; fluorene; benzofuran; or benzothiophene.

According to one embodiment of the present specification, R is hydrogen.

According to one embodiment of the present specification, R and R103 bond to each other to form a substituted or unsubstituted ring.

According to one embodiment of the present specification, R and R103 bond to each other to form a substituted or unsubstituted monocyclic or polycyclic aromatic hydrocarbon ring having 6 to 30 carbon atoms.

According to one embodiment of the present specification, R and R103 bond to each other to form a substituted or unsubstituted monocyclic or polycyclic aromatic hydrocarbon ring having 6 to 20 carbon atoms.

According to one embodiment of the present specification, R and R103 bond to each other to form a monocyclic or polycyclic aromatic hydrocarbon ring having 6 to 30 carbon atoms.

According to one embodiment of the present specification, R and R103 bond to each other to form a monocyclic or polycyclic aromatic hydrocarbon ring having 6 to 20 carbon atoms.

According to one embodiment of the present specification, R and R103 bond to each other to form a benzene ring.

According to one embodiment of the present specification, the compound of Chemical Formula 1 is any one compound selected from among the following compounds:

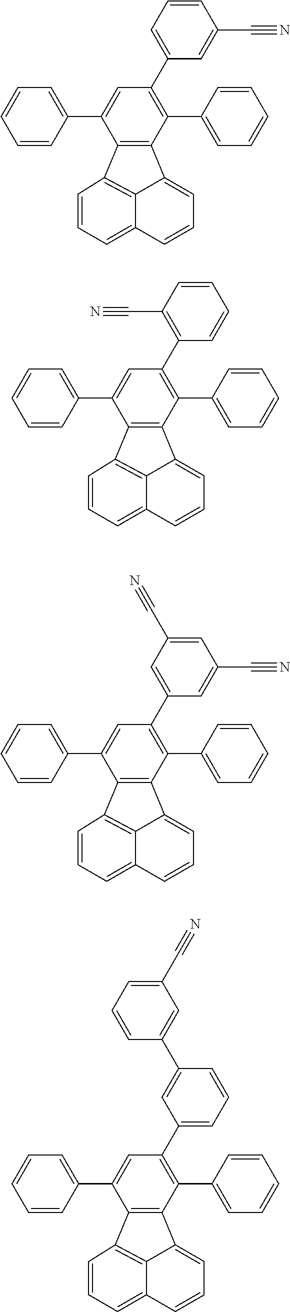

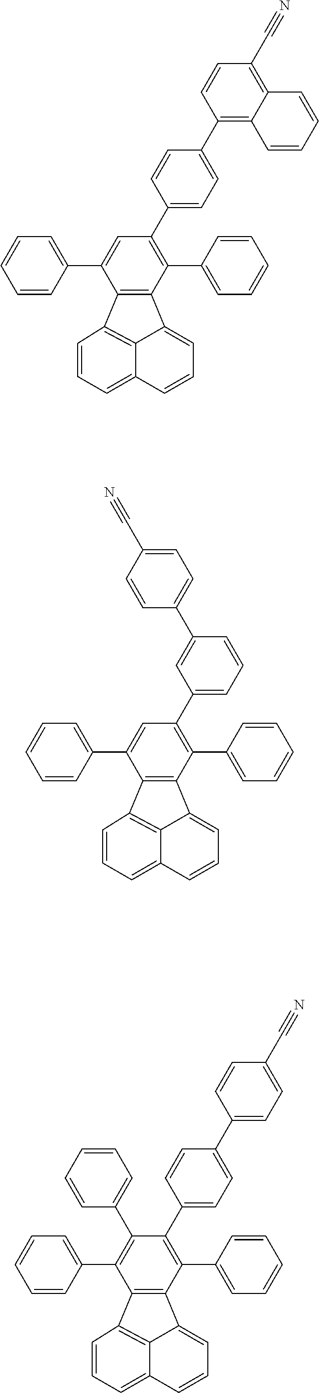 [SMILES: N#Cc1ccc(-c2ccc(-c3cc(-c4ccccc4)c4c(c3-c3ccccc3)-c3cccc5cccc-4c35)cc2)c2ccccc12 ; N#Cc1ccc(-c2cccc(-c3cc(-c4ccccc4)c4c(c3-c3ccccc3)-c3cccc5cccc-4c35)c2)cc1 ; N#Cc1ccc(-c2ccc(-c3c(-c4ccccc4)c(-c4ccccc4)c4c(c3-c3ccccc3)-c3cccc5cccc-4c35)cc2)cc1]

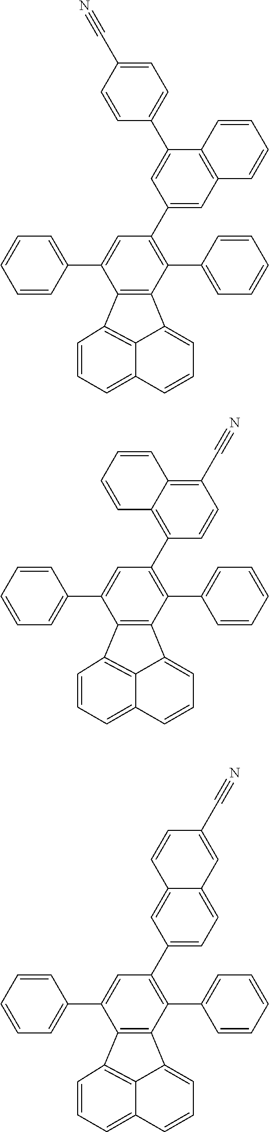 [SMILES: N#Cc1ccc(-c2cc(-c3cc(-c4ccccc4)c4c(c3-c3ccccc3)-c3cccc5cccc-4c35)cc3ccccc23)cc1 ; N#Cc1ccc(-c2cc(-c3ccccc3)c3c(c2-c2ccccc2)-c2cccc4cccc-3c24)c2ccccc12 ; N#Cc1ccc2cc(-c3cc(-c4ccccc4)c4c(c3-c3ccccc3)-c3cccc5cccc-4c35)ccc2c1]

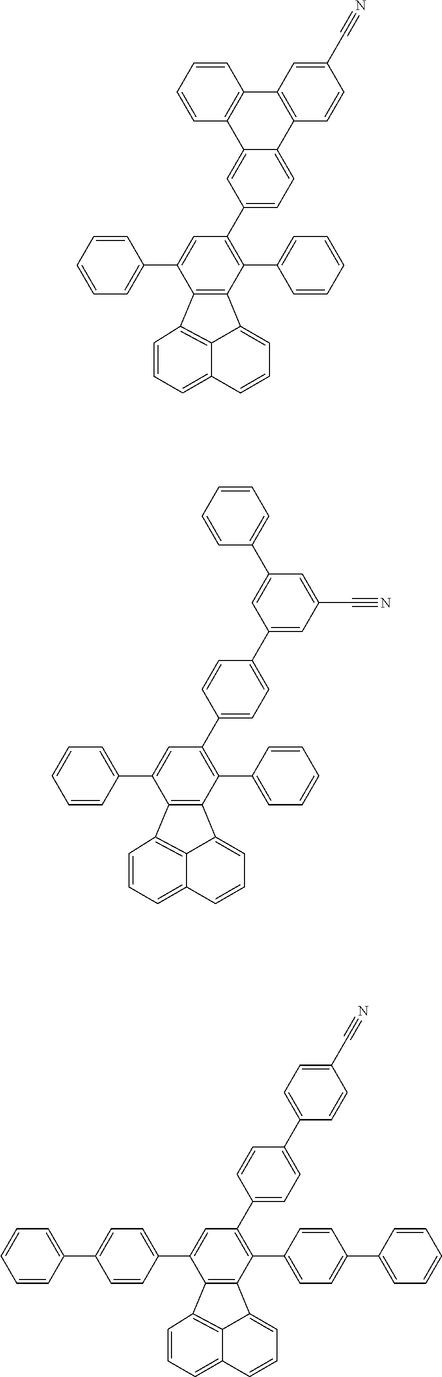 [SMILES: N#Cc1ccc2c3ccc(-c4cc(-c5ccccc5)c5c(c4-c4ccccc4)-c4cccc6cccc-5c46)cc3c3ccccc3c2c1 ; N#Cc1cc(-c2ccccc2)cc(-c2ccc(-c3cc(-c4ccccc4)c4c(c3-c3ccccc3)-c3cccc5cccc-4c35)cc2)c1 ; N#Cc1ccc(-c2ccc(-c3cc(-c4ccc(-c5ccccc5)cc4)c4c(c3-c3ccc(-c5ccccc5)cc3)-c3cccc5cccc-4c35)cc2)cc1]

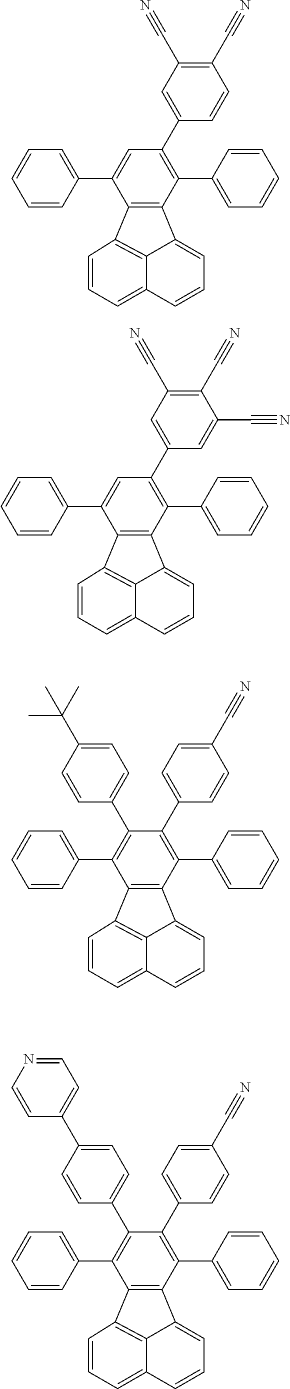

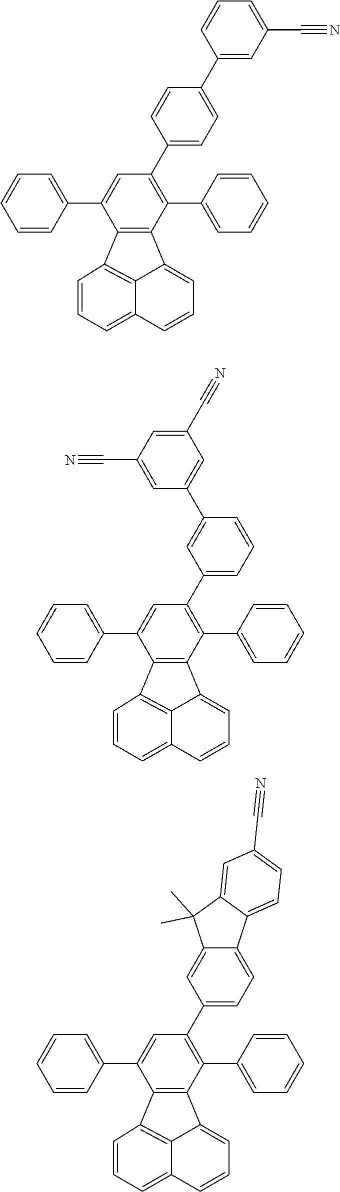 [SMILES: N#Cc1cccc(-c2ccc(-c3cc(-c4ccccc4)c4c(c3-c3ccccc3)-c3cccc5cccc-4c35)cc2)c1 ; N#Cc1cc(C#N)cc(-c2cccc(-c3cc(-c4ccccc4)c4c(c3-c3ccccc3)-c3cccc5cccc-4c35)c2)c1 ; CC1(C)c2cc(C#N)ccc2-c2ccc(-c3cc(-c4ccccc4)c4c(c3-c3ccccc3)-c3cccc5cccc-4c35)cc21]

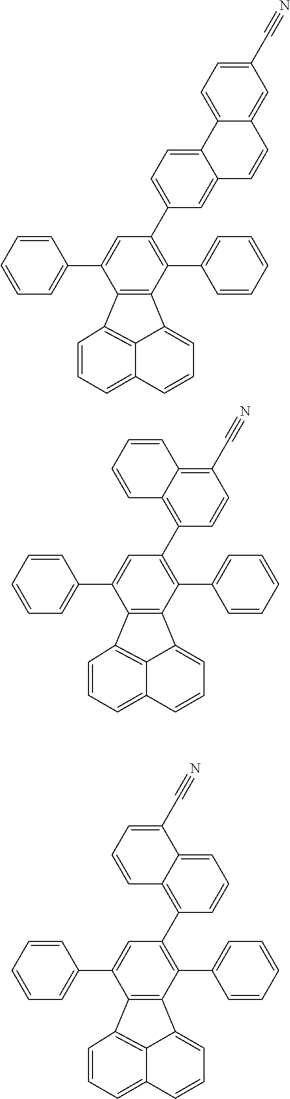

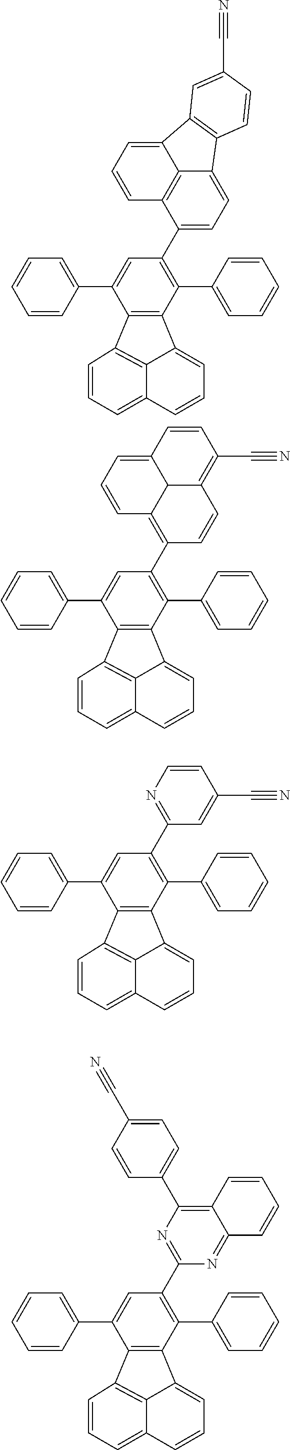

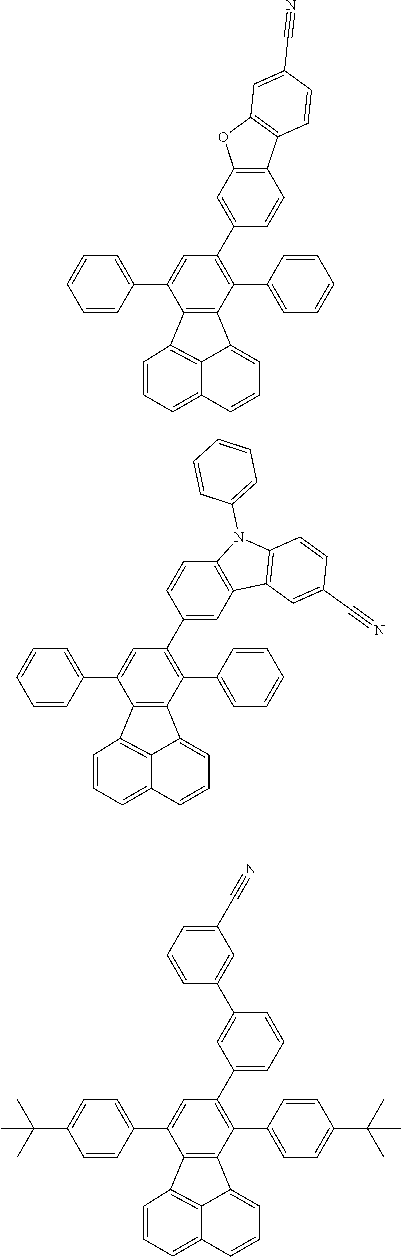

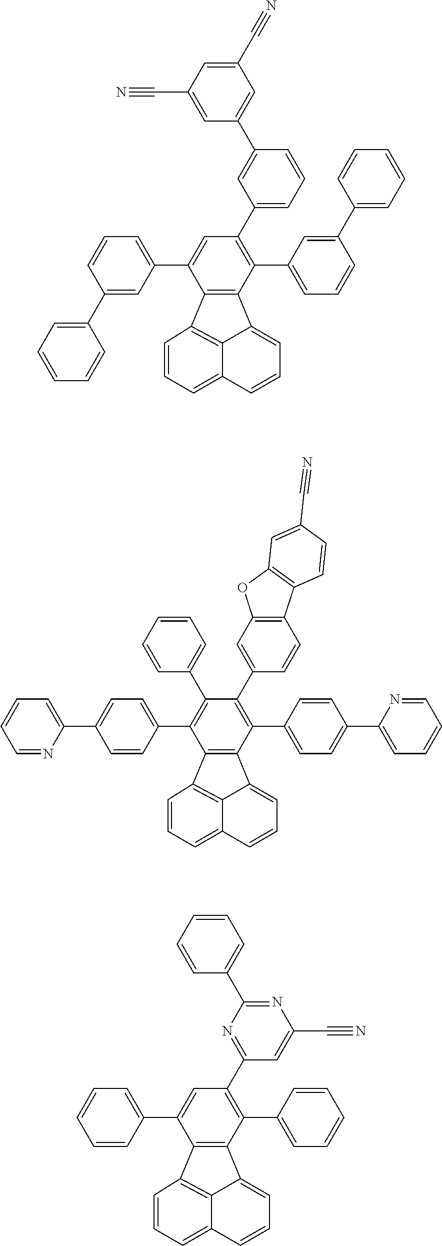 [SMILES: N#Cc1cc(C#N)cc(-c2cccc(-c3cc(-c4cccc(-c5ccccc5)c4)c4c(c3-c3cccc(-c5ccccc5)c3)-c3cccc5cccc-4c35)c2)c1 ; N#Cc1ccc2c(c1)oc1cc(-c3c(-c4ccccc4)c(-c4ccc(-c5ccccn5)cc4)c4c(c3-c3ccc(-c5ccccn5)cc3)-c3cccc5cccc-4c35)ccc12 ; N#Cc1cc(-c2cc(-c3ccccc3)c3c(c2-c2ccccc2)-c2cccc4cccc-3c24)nc(-c2ccccc2)n1]

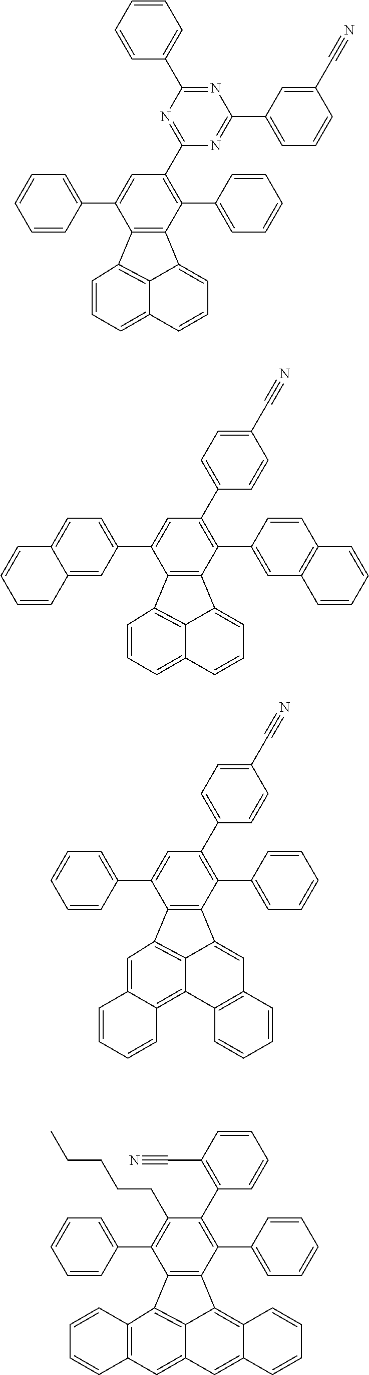 [SMILES: N#Cc1cccc(-c2nc(-c3ccccc3)nc(-c3cc(-c4ccccc4)c4c(c3-c3ccccc3)-c3cccc5cccc-4c35)n2)c1 ; N#Cc1ccc(-c2cc(-c3ccc4ccccc4c3)c3c(c2-c2ccc4ccccc4c2)-c2cccc4cccc-3c24)cc1 ; N#Cc1ccc(-c2cc(-c3ccccc3)c3c(c2-c2ccccc2)-c2cc4ccccc4c4c2c-3cc2ccccc24)cc1 ; CCCCCc1c(-c2ccccc2)c2c(c(-c3ccccc3)c1-c1ccccc1C#N)-c1c3ccccc3cc3cc4ccccc4c-2c13]

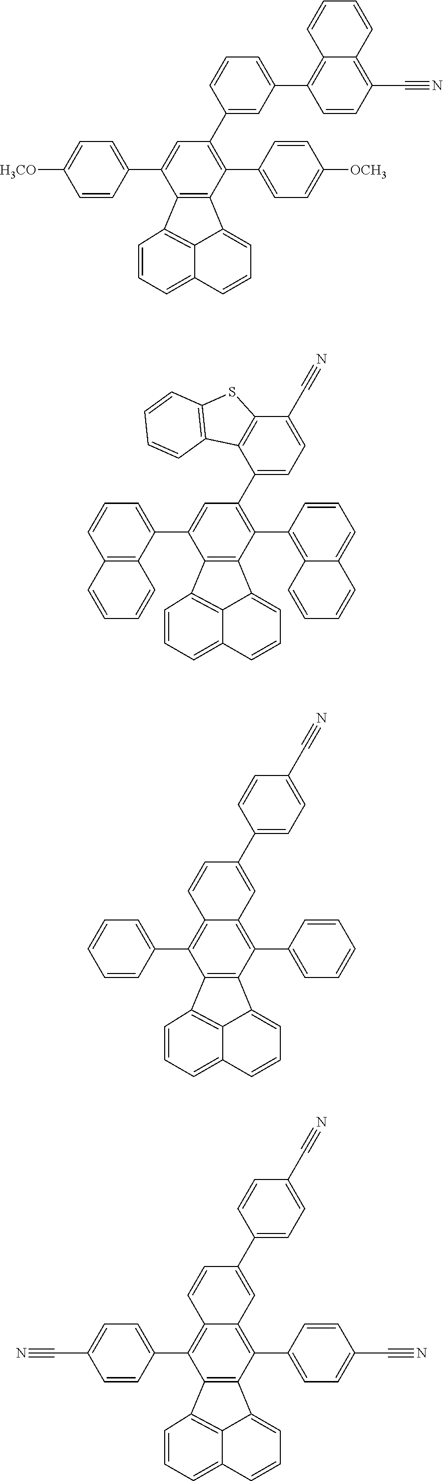 [SMILES: COc1ccc(-c2cc(-c3cccc(-c4ccc(C#N)c5ccccc45)c3)c(-c3ccc(OC)cc3)c3c2-c2cccc4cccc-3c24)cc1 ; N#Cc1ccc(-c2cc(-c3cccc4ccccc34)c3c(c2-c2cccc4ccccc24)-c2cccc4cccc-3c24)c2c1sc1ccccc12 ; N#Cc1ccc(-c2ccc3c(-c4ccccc4)c4c(c(-c5ccccc5)c3c2)-c2cccc3cccc-4c23)cc1 ; N#Cc1ccc(-c2ccc3c(-c4ccc(C#N)cc4)c4c(c(-c5ccc(C#N)cc5)c3c2)-c2cccc3cccc-4c23)cc1]

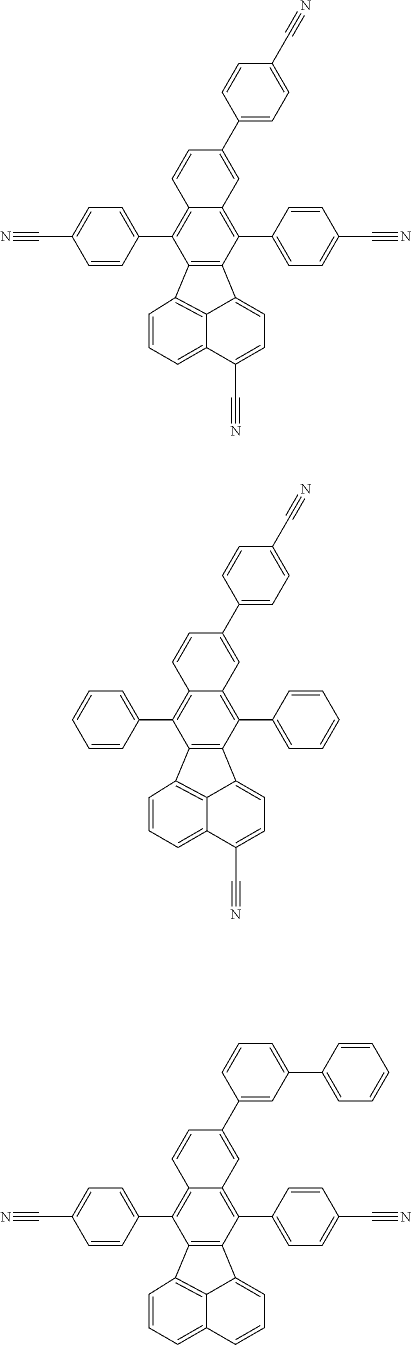 [SMILES: N#Cc1ccc(-c2ccc3c(-c4ccc(C#N)cc4)c4c(c(-c5ccc(C#N)cc5)c3c2)-c2ccc(C#N)c3cccc-4c23)cc1 ; N#Cc1ccc(-c2ccc3c(-c4ccccc4)c4c(c(-c5ccccc5)c3c2)-c2ccc(C#N)c3cccc-4c23)cc1 ; N#Cc1ccc(-c2c3c(c(-c4ccc(C#N)cc4)c4cc(-c5cccc(-c6ccccc6)c5)ccc24)-c2cccc4cccc-3c24)cc1]

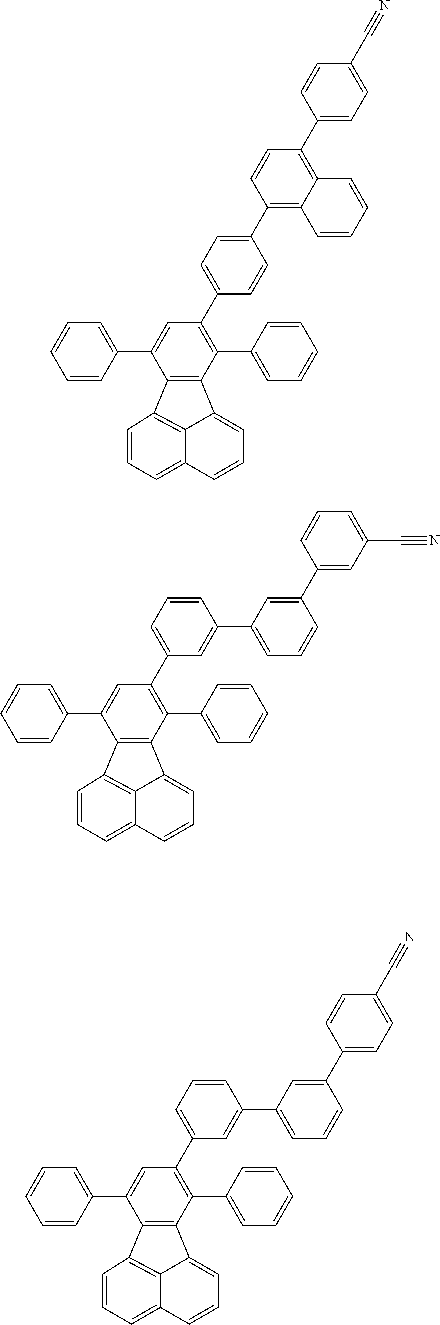 [SMILES: N#Cc1ccc(-c2ccc(-c3ccc(-c4cc(-c5ccccc5)c5c(c4-c4ccccc4)-c4cccc6cccc-5c46)cc3)c3ccccc23)cc1 ; N#Cc1cccc(-c2cccc(-c3cccc(-c4cc(-c5ccccc5)c5c(c4-c4ccccc4)-c4cccc6cccc-5c46)c3)c2)c1 ; N#Cc1ccc(-c2cccc(-c3cccc(-c4cc(-c5ccccc5)c5c(c4-c4ccccc4)-c4cccc6cccc-5c46)c3)c2)cc1]

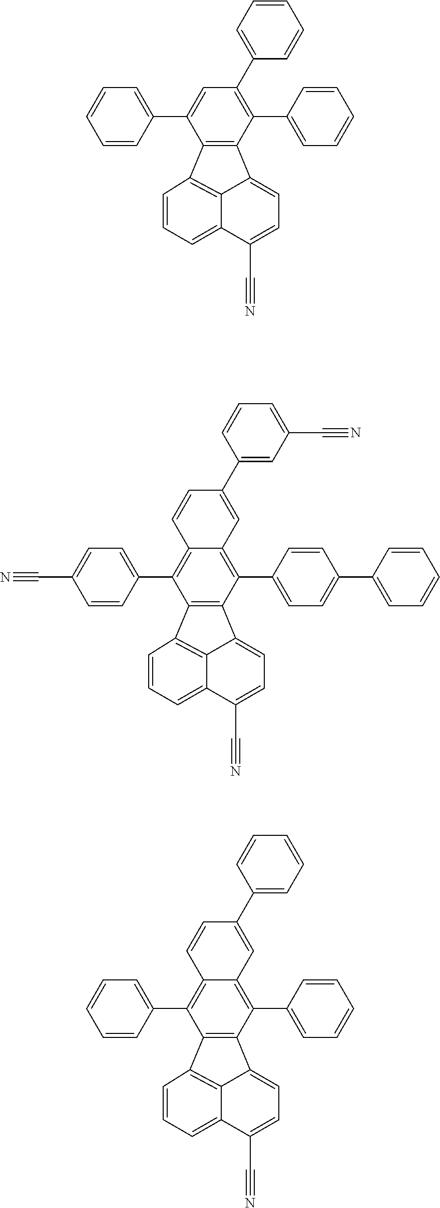 [SMILES: N#Cc1ccc2c3c(cccc13)-c1c(-c3ccccc3)cc(-c3ccccc3)c(-c3ccccc3)c1-2 ; N#Cc1ccc(-c2c3c(c(-c4ccc(-c5ccccc5)cc4)c4cc(-c5cccc(C#N)c5)ccc24)-c2ccc(C#N)c4cccc-3c24)cc1 ; N#Cc1ccc2c3c(cccc13)-c1c-2c(-c2ccccc2)c2cc(-c3ccccc3)ccc2c1-c1ccccc1]

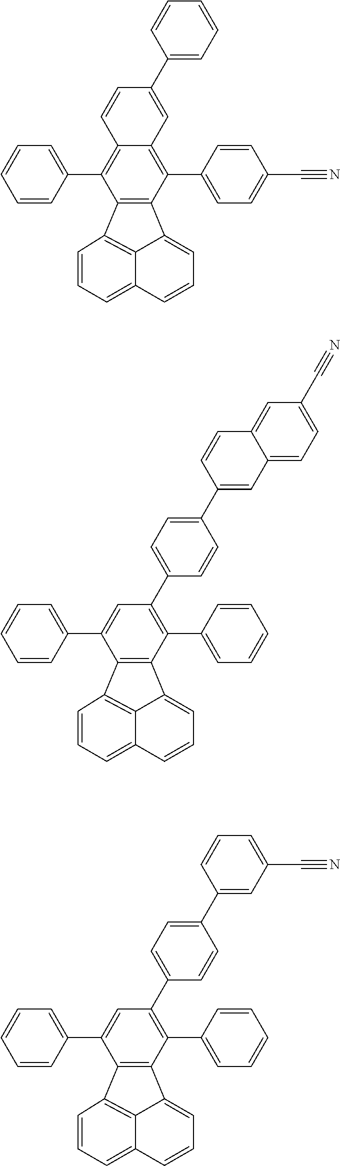 [SMILES: N#Cc1ccc(-c2c3c(c(-c4ccccc4)c4ccc(-c5ccccc5)cc24)-c2cccc4cccc-3c24)cc1 ; N#Cc1ccc2cc(-c3ccc(-c4cc(-c5ccccc5)c5c(c4-c4ccccc4)-c4cccc6cccc-5c46)cc3)ccc2c1 ; N#Cc1cccc(-c2ccc(-c3cc(-c4ccccc4)c4c(c3-c3ccccc3)-c3cccc5cccc-4c35)cc2)c1]

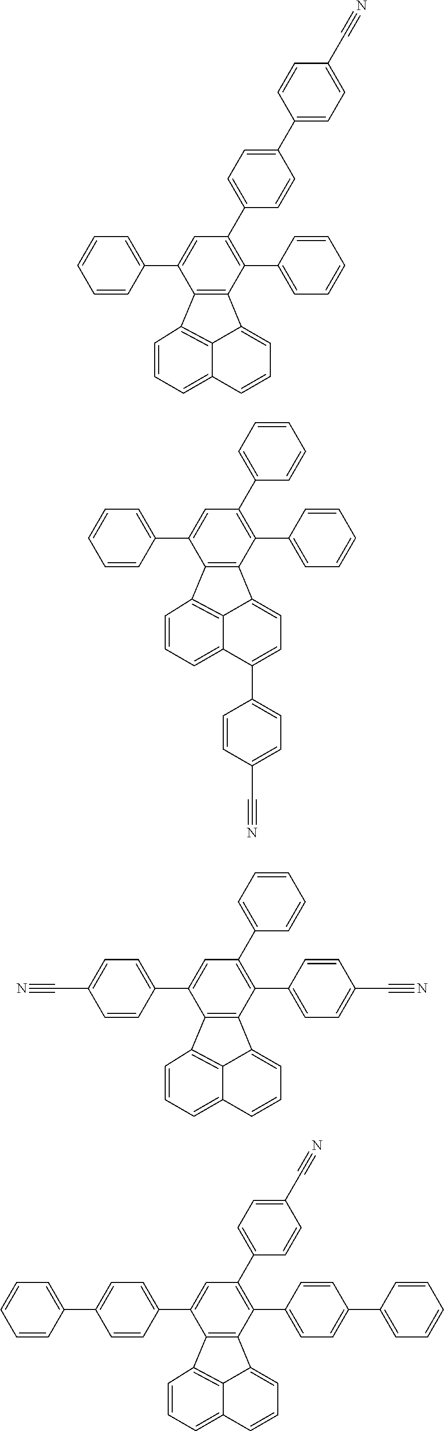

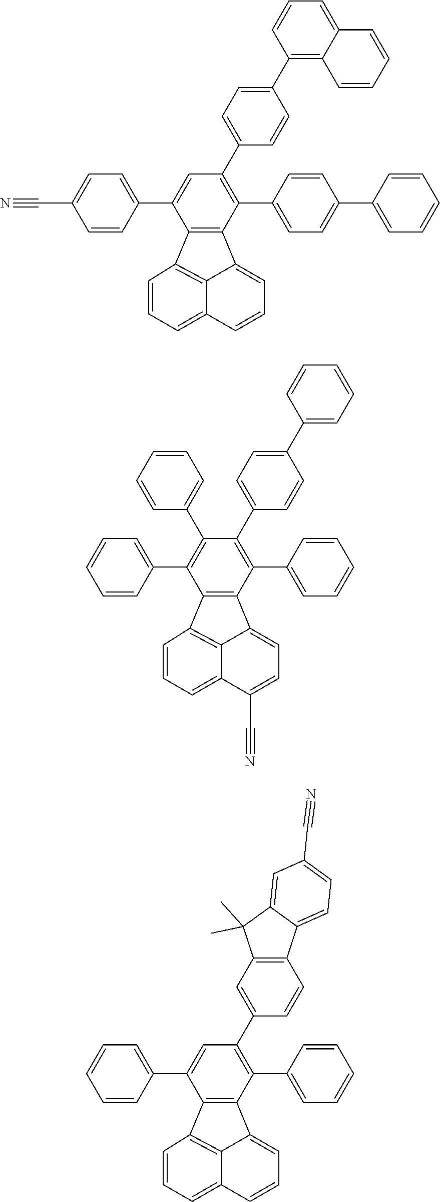 [SMILES: N#Cc1ccc(-c2cc(-c3ccc(-c4cccc5ccccc45)cc3)c(-c3ccc(-c4ccccc4)cc3)c3c2-c2cccc4cccc-3c24)cc1 ; N#Cc1ccc2c3c(cccc13)-c1c(-c3ccccc3)c(-c3ccccc3)c(-c3ccc(-c4ccccc4)cc3)c(-c3ccccc3)c1-2 ; CC1(C)c2cc(C#N)ccc2-c2ccc(-c3cc(-c4ccccc4)c4c(c3-c3ccccc3)-c3cccc5cccc-4c35)cc21]

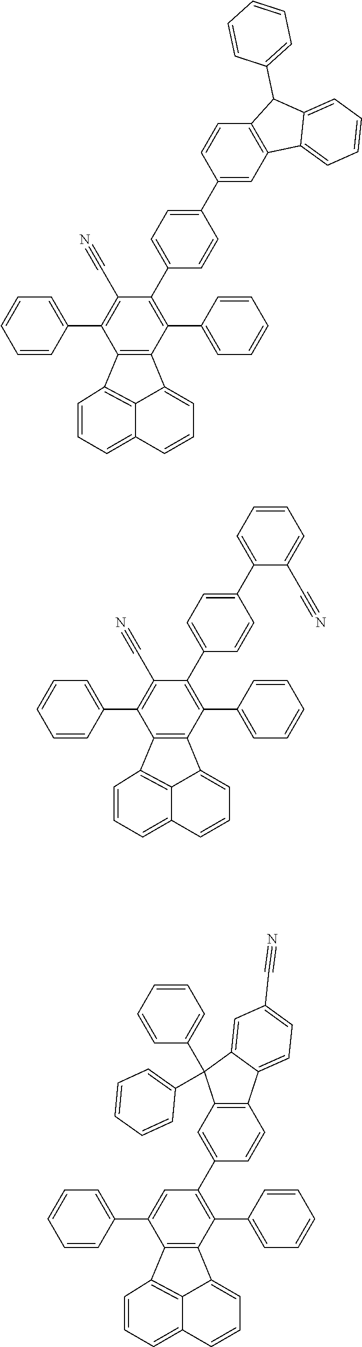 [SMILES: N#Cc1c(-c2ccc(-c3ccc4c(c3)-c3ccccc3C4c3ccccc3)cc2)c(-c2ccccc2)c2c(c1-c1ccccc1)-c1cccc3cccc-2c13 ; N#Cc1ccccc1-c1ccc(-c2c(C#N)c(-c3ccccc3)c3c(c2-c2ccccc2)-c2cccc4cccc-3c24)cc1 ; N#Cc1ccc2c(c1)C(c1ccccc1)(c1ccccc1)c1cc(-c3cc(-c4ccccc4)c4c(c3-c3ccccc3)-c3cccc5cccc-4c35)ccc1-2]

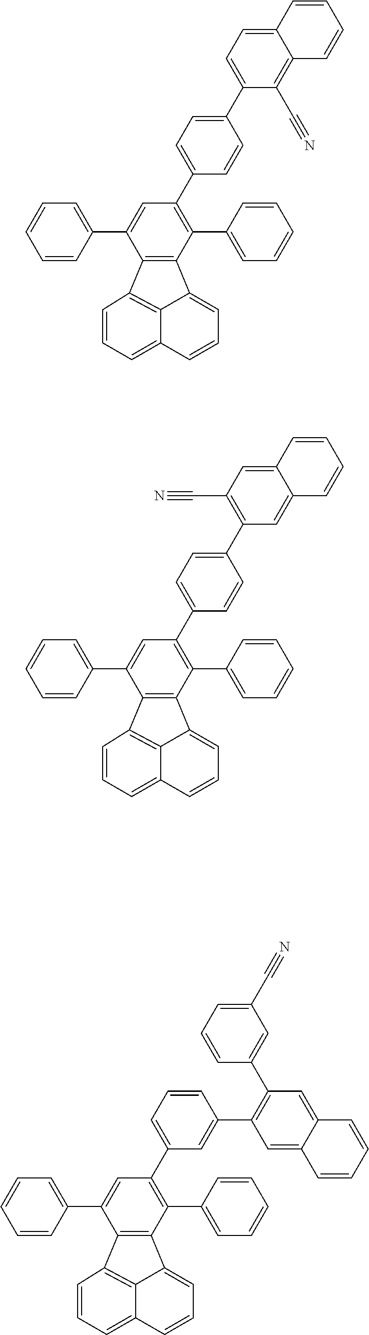 [SMILES: N#Cc1c(-c2ccc(-c3cc(-c4ccccc4)c4c(c3-c3ccccc3)-c3cccc5cccc-4c35)cc2)ccc2ccccc12 ; N#Cc1cc2ccccc2cc1-c1ccc(-c2cc(-c3ccccc3)c3c(c2-c2ccccc2)-c2cccc4cccc-3c24)cc1 ; N#Cc1cccc(-c2cc3ccccc3cc2-c2cccc(-c3cc(-c4ccccc4)c4c(c3-c3ccccc3)-c3cccc5cccc-4c35)c2)c1]

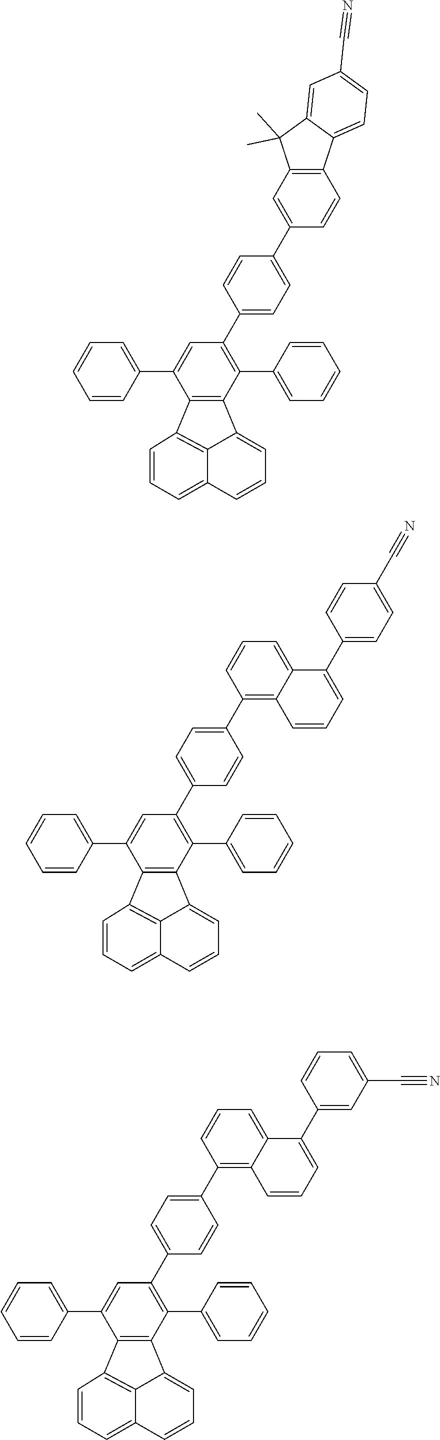 [SMILES: CC1(C)c2cc(C#N)ccc2-c2ccc(-c3ccc(-c4cc(-c5ccccc5)c5c(c4-c4ccccc4)-c4cccc6cccc-5c46)cc3)cc21 ; N#Cc1ccc(-c2cccc3c(-c4ccc(-c5cc(-c6ccccc6)c6c(c5-c5ccccc5)-c5cccc7cccc-6c57)cc4)cccc23)cc1 ; N#Cc1cccc(-c2cccc3c(-c4ccc(-c5cc(-c6ccccc6)c6c(c5-c5ccccc5)-c5cccc7cccc-6c57)cc4)cccc23)c1]

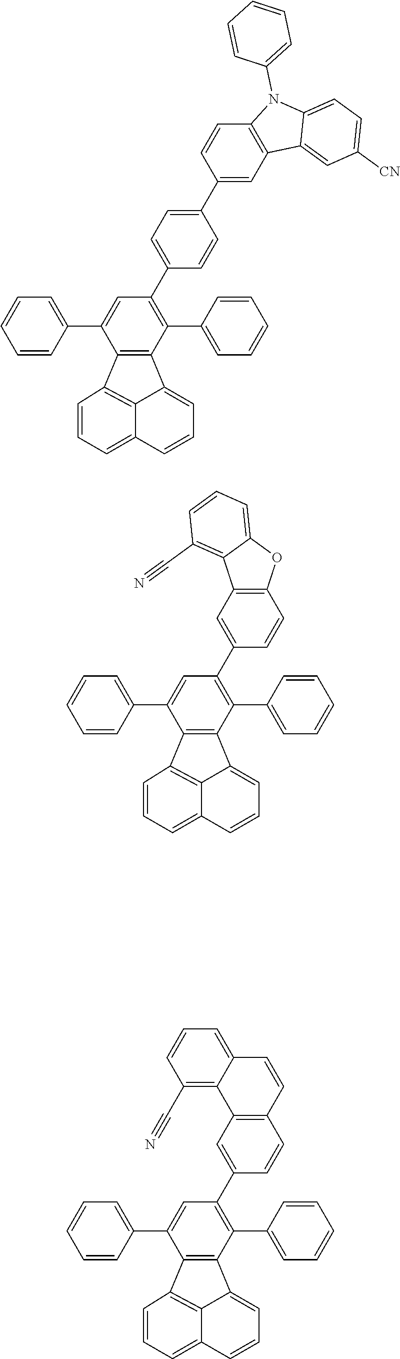 [SMILES: N#Cc1ccc2c(c1)c1cc(-c3ccc(-c4cc(-c5ccccc5)c5c(c4-c4ccccc4)-c4cccc6cccc-5c46)cc3)ccc1n2-c1ccccc1 ; N#Cc1cccc2oc3ccc(-c4cc(-c5ccccc5)c5c(c4-c4ccccc4)-c4cccc6cccc-5c46)cc3c12 ; N#Cc1cccc2ccc3ccc(-c4cc(-c5ccccc5)c5c(c4-c4ccccc4)-c4cccc6cccc-5c46)cc3c12]

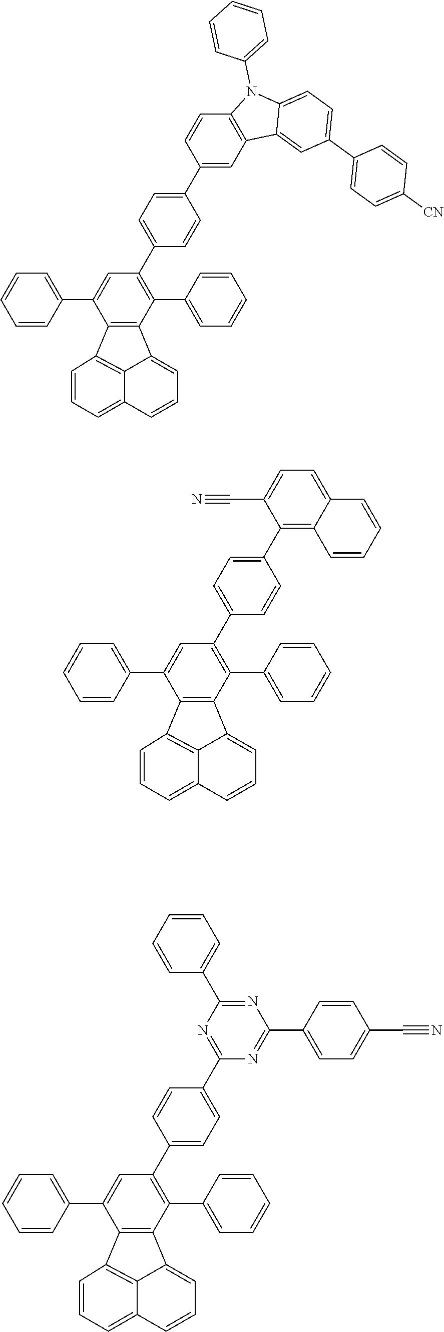 [SMILES: N#Cc1ccc(-c2ccc3c(c2)c2cc(-c4ccc(-c5cc(-c6ccccc6)c6c(c5-c5ccccc5)-c5cccc7cccc-6c57)cc4)ccc2n3-c2ccccc2)cc1 ; N#Cc1ccc2ccccc2c1-c1ccc(-c2cc(-c3ccccc3)c3c(c2-c2ccccc2)-c2cccc4cccc-3c24)cc1 ; N#Cc1ccc(-c2nc(-c3ccccc3)nc(-c3ccc(-c4cc(-c5ccccc5)c5c(c4-c4ccccc4)-c4cccc6cccc-5c46)cc3)n2)cc1]

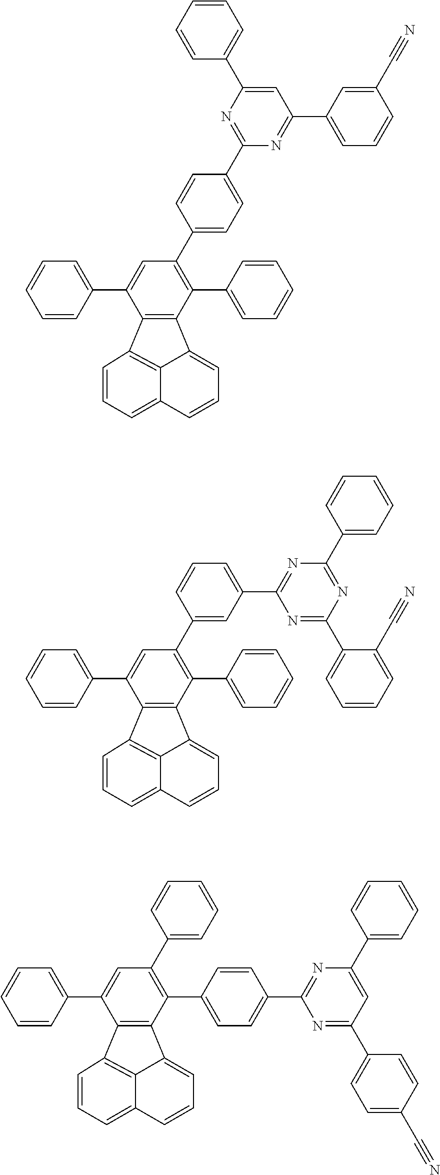 [SMILES: N#Cc1cccc(-c2cc(-c3ccccc3)nc(-c3ccc(-c4cc(-c5ccccc5)c5c(c4-c4ccccc4)-c4cccc6cccc-5c46)cc3)n2)c1 ; N#Cc1ccccc1-c1nc(-c2ccccc2)nc(-c2cccc(-c3cc(-c4ccccc4)c4c(c3-c3ccccc3)-c3cccc5cccc-4c35)c2)n1 ; N#Cc1ccc(-c2cc(-c3ccccc3)nc(-c3ccc(-c4c(-c5ccccc5)cc(-c5ccccc5)c5c4-c4cccc6cccc-5c46)cc3)n2)cc1]

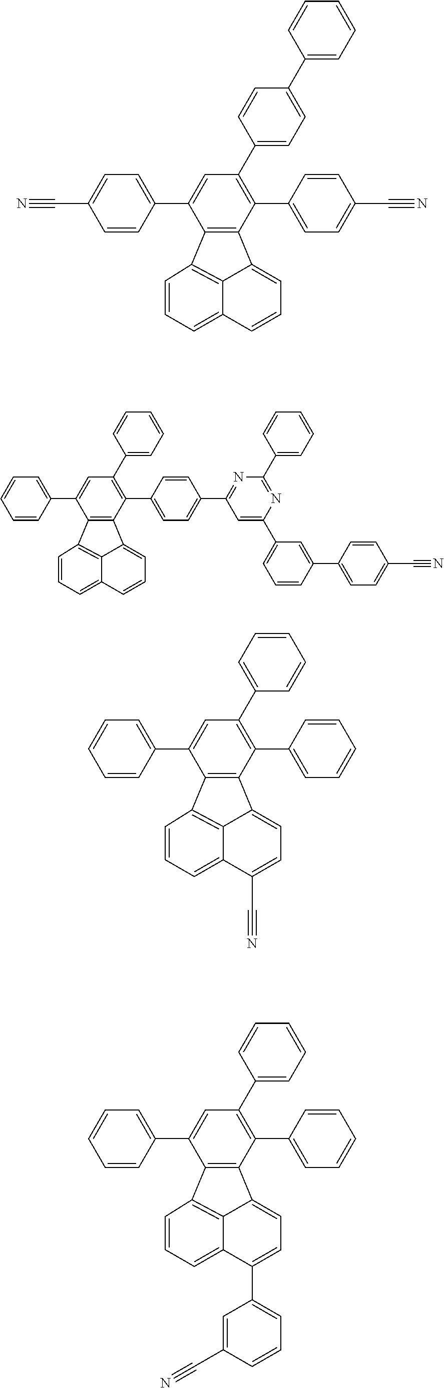 [SMILES: N#Cc1ccc(-c2cc(-c3ccc(-c4ccccc4)cc3)c(-c3ccc(C#N)cc3)c3c2-c2cccc4cccc-3c24)cc1 ; N#Cc1ccc(-c2cccc(-c3cc(-c4ccc(-c5c(-c6ccccc6)cc(-c6ccccc6)c6c5-c5cccc7cccc-6c57)cc4)nc(-c4ccccc4)n3)c2)cc1 ; N#Cc1ccc2c3c(cccc13)-c1c(-c3ccccc3)cc(-c3ccccc3)c(-c3ccccc3)c1-2 ; N#Cc1cccc(-c2ccc3c4c(cccc24)-c2c(-c4ccccc4)cc(-c4ccccc4)c(-c4ccccc4)c2-3)c1]

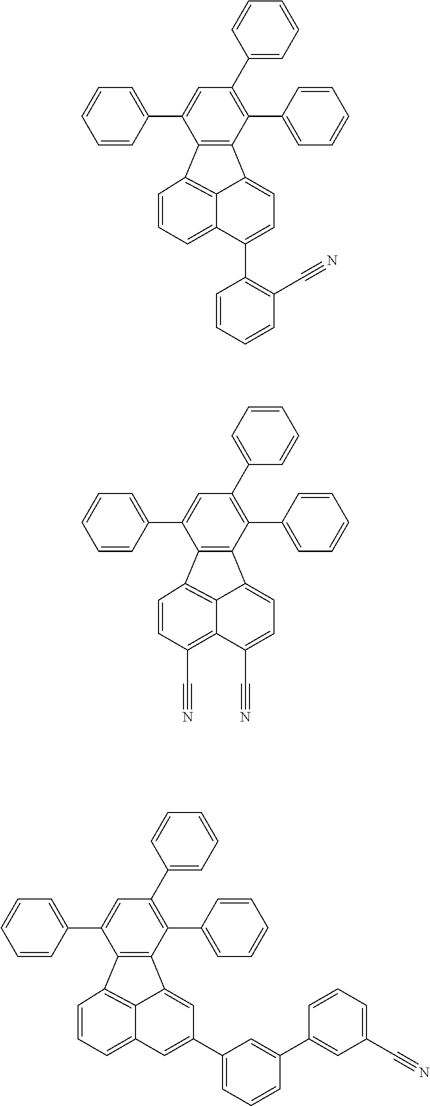 [SMILES: N#Cc1ccccc1-c1ccc2c3c(cccc13)-c1c(-c3ccccc3)cc(-c3ccccc3)c(-c3ccccc3)c1-2 ; N#Cc1ccc2c3c(ccc(C#N)c13)-c1c(-c3ccccc3)c(-c3ccccc3)cc(-c3ccccc3)c1-2 ; N#Cc1cccc(-c2cccc(-c3cc4c5c(cccc5c3)-c3c(-c5ccccc5)cc(-c5ccccc5)c(-c5ccccc5)c3-4)c2)c1]

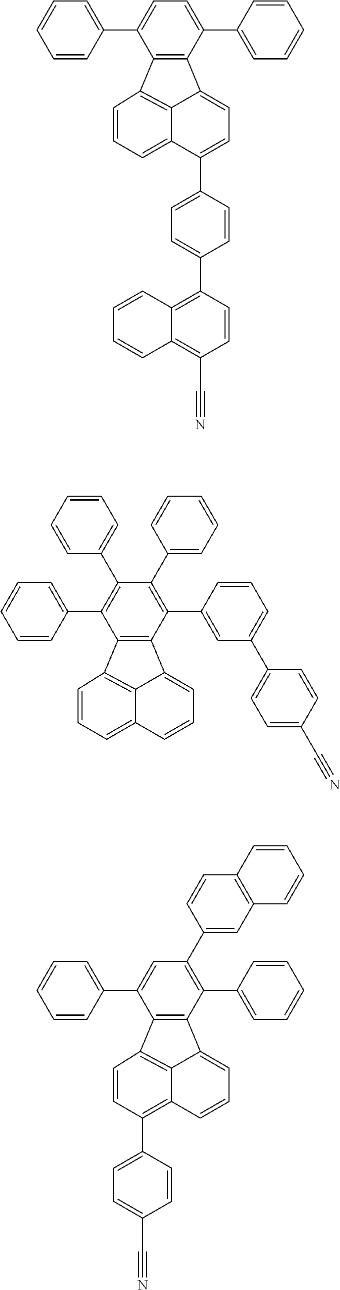 [SMILES: N#Cc1ccc(-c2ccc(-c3ccc4c5c(cccc35)-c3c(-c5ccccc5)ccc(-c5ccccc5)c3-4)cc2)c2ccccc12 ; N#Cc1ccc(-c2cccc(-c3c(-c4ccccc4)c(-c4ccccc4)c(-c4ccccc4)c4c3-c3cccc5cccc-4c35)c2)cc1 ; N#Cc1ccc(-c2ccc3c4c(cccc24)-c2c(-c4ccccc4)c(-c4ccc5ccccc5c4)cc(-c4ccccc4)c2-3)cc1]

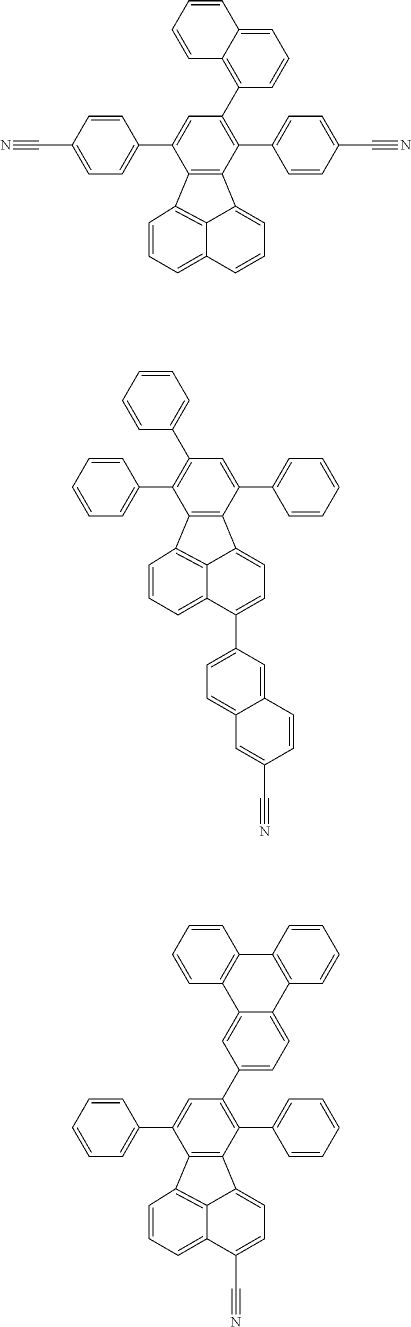 [SMILES: N#Cc1ccc(-c2cc(-c3cccc4ccccc34)c(-c3ccc(C#N)cc3)c3c2-c2cccc4cccc-3c24)cc1 ; N#Cc1ccc2cc(-c3ccc4c5c(cccc35)-c3c(-c5ccccc5)c(-c5ccccc5)cc(-c5ccccc5)c3-4)ccc2c1 ; N#Cc1ccc2c3c(cccc13)-c1c(-c3ccccc3)cc(-c3ccc4c5ccccc5c5ccccc5c4c3)c(-c3ccccc3)c1-2]

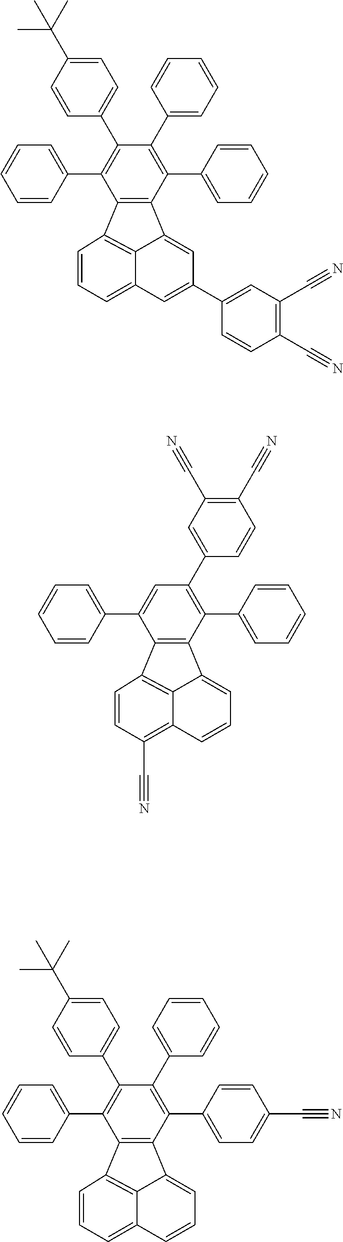 [SMILES: CC(C)(C)c1ccc(-c2c(-c3ccccc3)c(-c3ccccc3)c3c(c2-c2ccccc2)-c2cccc4cc(-c5ccc(C#N)c(C#N)c5)cc-3c24)cc1 ; N#Cc1ccc(-c2cc(-c3ccccc3)c3c(c2-c2ccccc2)-c2cccc4c(C#N)ccc-3c24)cc1C#N ; CC(C)(C)c1ccc(-c2c(-c3ccccc3)c(-c3ccc(C#N)cc3)c3c(c2-c2ccccc2)-c2cccc4cccc-3c24)cc1]

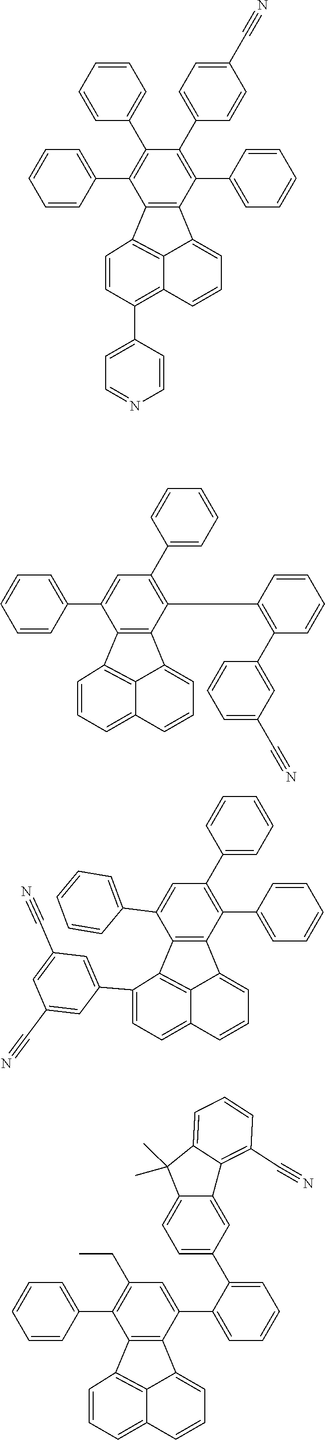

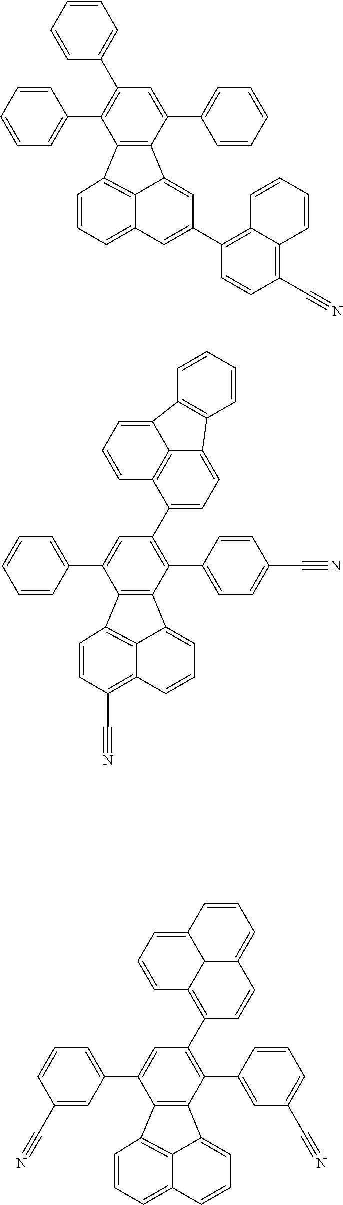 [SMILES: N#Cc1ccc(-c2cc3c4c(cccc4c2)-c2c(-c4ccccc4)c(-c4ccccc4)cc(-c4ccccc4)c2-3)c2ccccc12 ; N#Cc1ccc(-c2c(-c3ccc4c5c(cccc35)-c3ccccc3-4)cc(-c3ccccc3)c3c2-c2cccc4c(C#N)ccc-3c24)cc1 ; N#Cc1cccc(-c2cc(C3=C4C=CC=C5C=CC=C(C=C3)C54)c(-c3cccc(C#N)c3)c3c2-c2cccc4cccc-3c24)c1]

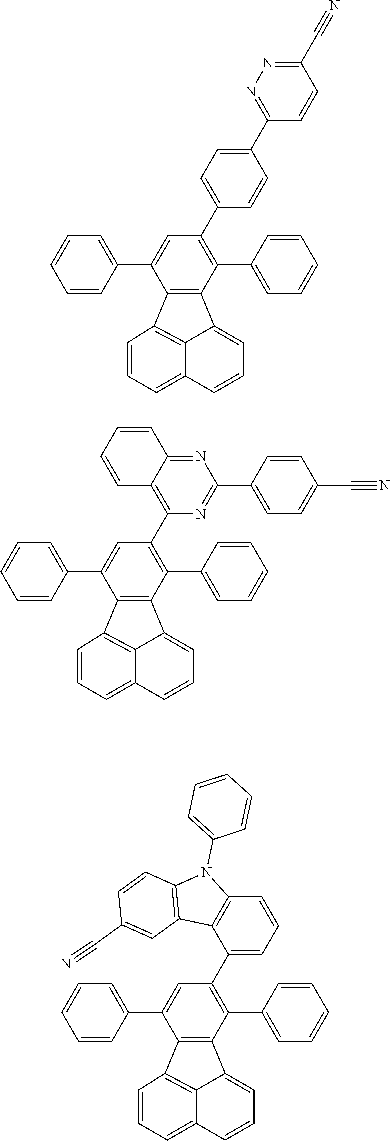

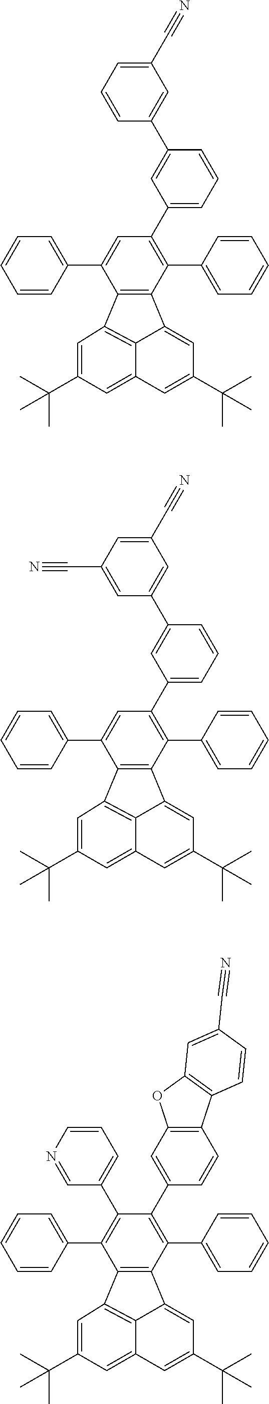 [SMILES: CC(C)(C)c1cc2c3c(cc(C(C)(C)C)cc3c1)-c1c(-c3ccccc3)c(-c3cccc(-c4cccc(C#N)c4)c3)cc(-c3ccccc3)c1-2 ; CC(C)(C)c1cc2c3c(cc(C(C)(C)C)cc3c1)-c1c(-c3ccccc3)c(-c3cccc(-c4cc(C#N)cc(C#N)c4)c3)cc(-c3ccccc3)c1-2 ; CC(C)(C)c1cc2c3c(cc(C(C)(C)C)cc3c1)-c1c(-c3ccccc3)c(-c3ccc4c(c3)oc3cc(C#N)ccc34)c(-c3cccnc3)c(-c3ccccc3)c1-2]

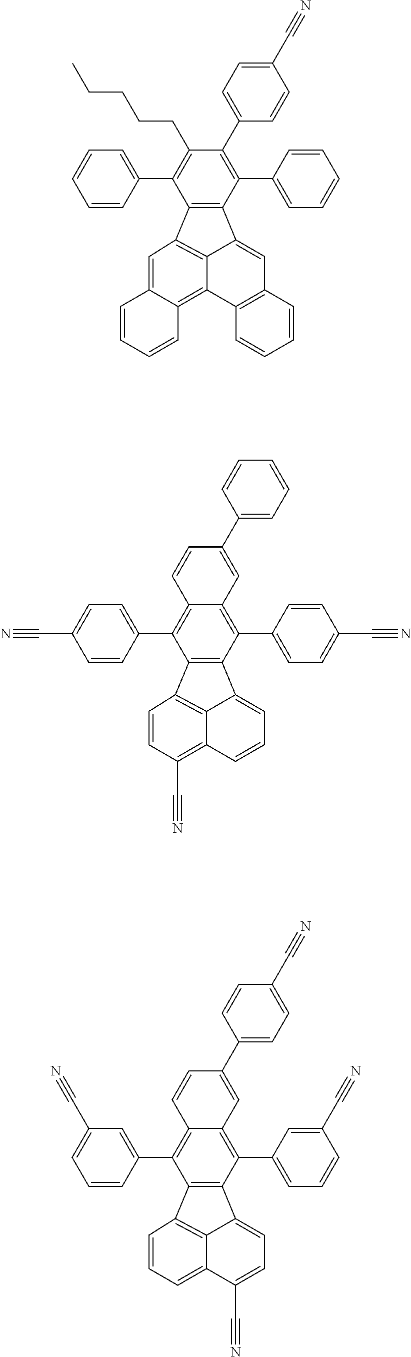 [SMILES: CCCCCc1c(-c2ccc(C#N)cc2)c(-c2ccccc2)c2c(c1-c1ccccc1)-c1cc3ccccc3c3c1c-2cc1ccccc13 ; N#Cc1ccc(-c2c3c(c(-c4ccc(C#N)cc4)c4cc(-c5ccccc5)ccc24)-c2cccc4c(C#N)ccc-3c24)cc1 ; N#Cc1ccc(-c2ccc3c(-c4cccc(C#N)c4)c4c(c(-c5cccc(C#N)c5)c3c2)-c2ccc(C#N)c3cccc-4c23)cc1]

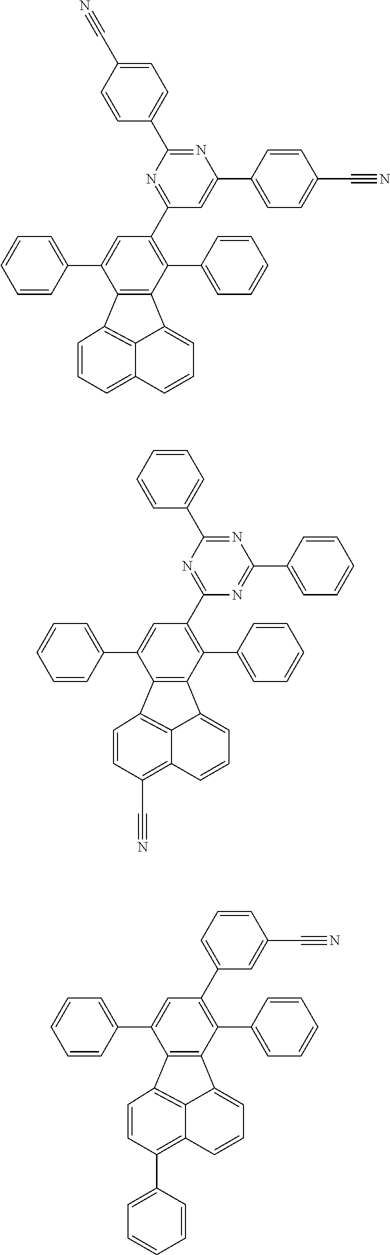 [SMILES: N#Cc1ccc(-c2cc(-c3cc(-c4ccccc4)c4c(c3-c3ccccc3)-c3cccc5cccc-4c35)nc(-c3ccc(C#N)cc3)n2)cc1 ; N#Cc1ccc2c3c(cccc13)-c1c(-c3ccccc3)c(-c3nc(-c4ccccc4)nc(-c4ccccc4)n3)cc(-c3ccccc3)c1-2 ; N#Cc1cccc(-c2cc(-c3ccccc3)c3c(c2-c2ccccc2)-c2cccc4c(-c5ccccc5)ccc-3c24)c1]

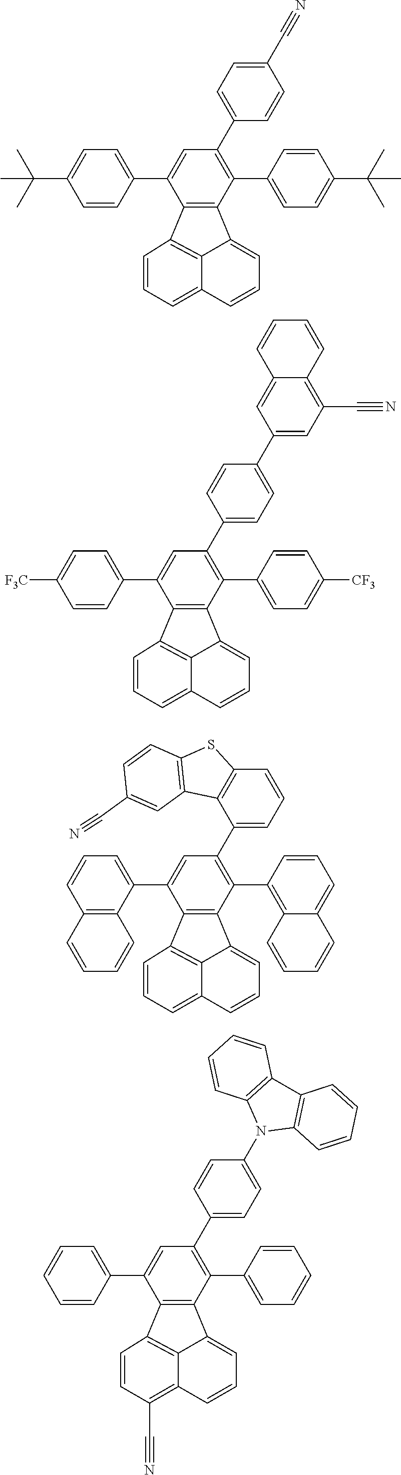

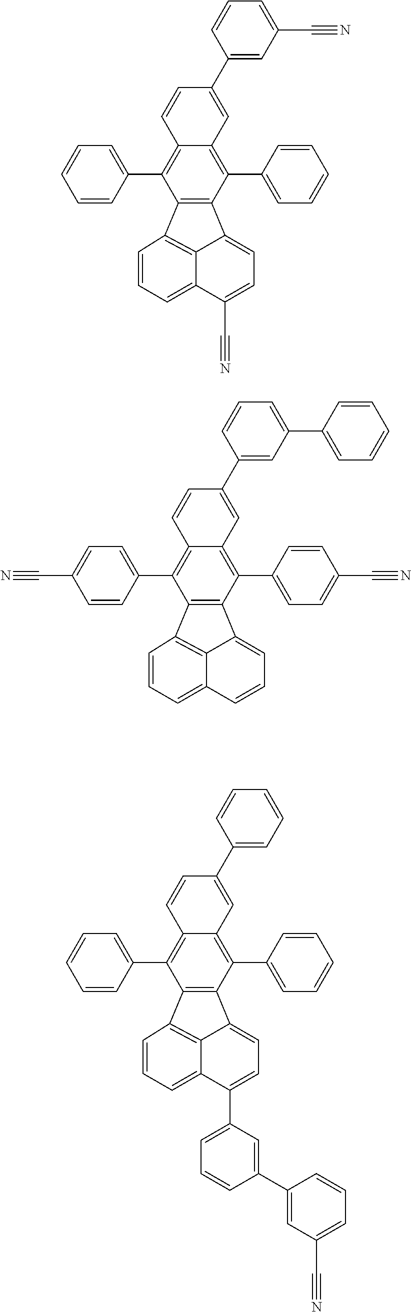

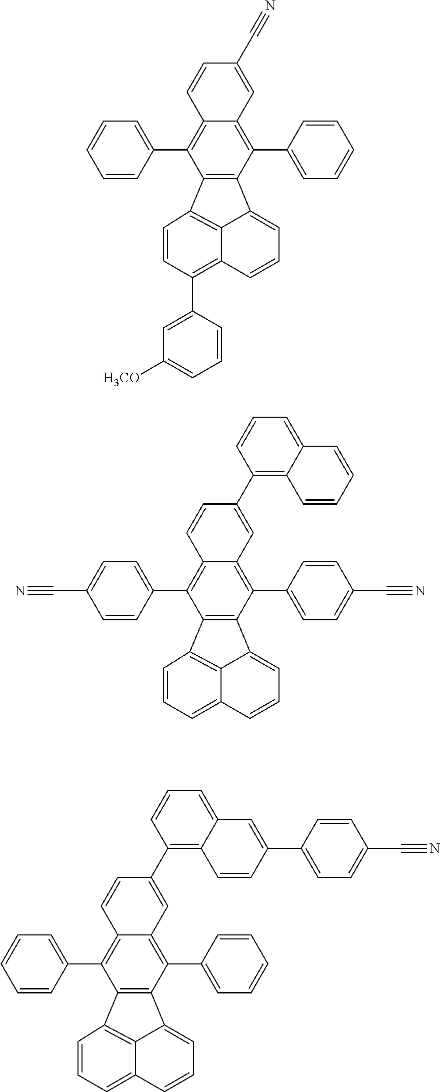 [SMILES: COc1cccc(-c2ccc3c4c(cccc24)-c2c-3c(-c3ccccc3)c3ccc(C#N)cc3c2-c2ccccc2)c1 ; N#Cc1ccc(-c2c3c(c(-c4ccc(C#N)cc4)c4cc(-c5cccc6ccccc56)ccc24)-c2cccc4cccc-3c24)cc1 ; N#Cc1ccc(-c2ccc3c(-c4ccc5c(-c6ccccc6)c6c(c(-c7ccccc7)c5c4)-c4cccc5cccc-6c45)cccc3c2)cc1]

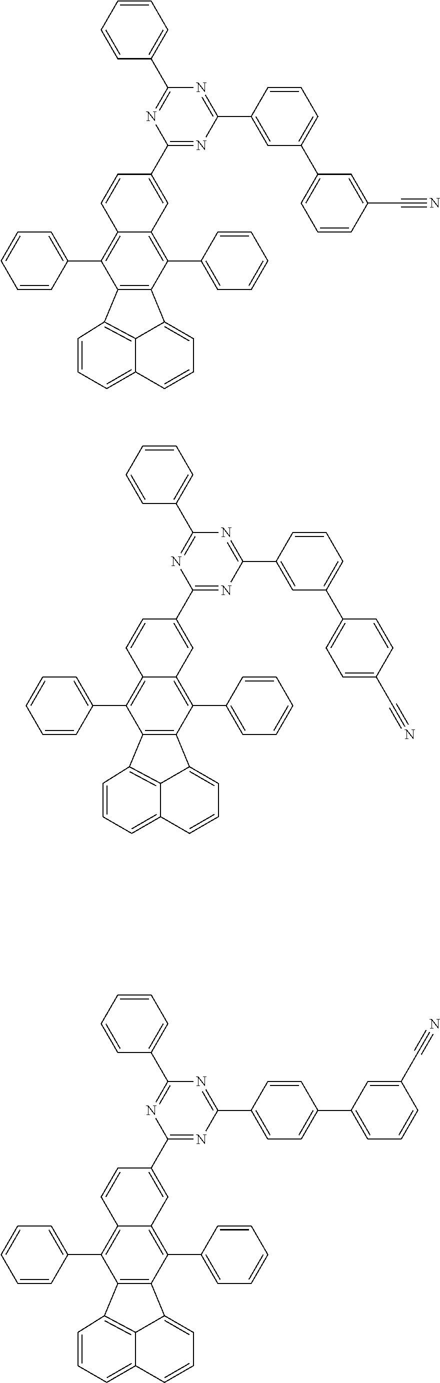 [SMILES: N#Cc1cccc(-c2cccc(-c3nc(-c4ccccc4)nc(-c4ccc5c(-c6ccccc6)c6c(c(-c7ccccc7)c5c4)-c4cccc5cccc-6c45)n3)c2)c1 ; N#Cc1ccc(-c2cccc(-c3nc(-c4ccccc4)nc(-c4ccc5c(-c6ccccc6)c6c(c(-c7ccccc7)c5c4)-c4cccc5cccc-6c45)n3)c2)cc1 ; N#Cc1cccc(-c2ccc(-c3nc(-c4ccccc4)nc(-c4ccc5c(-c6ccccc6)c6c(c(-c7ccccc7)c5c4)-c4cccc5cccc-6c45)n3)cc2)c1]

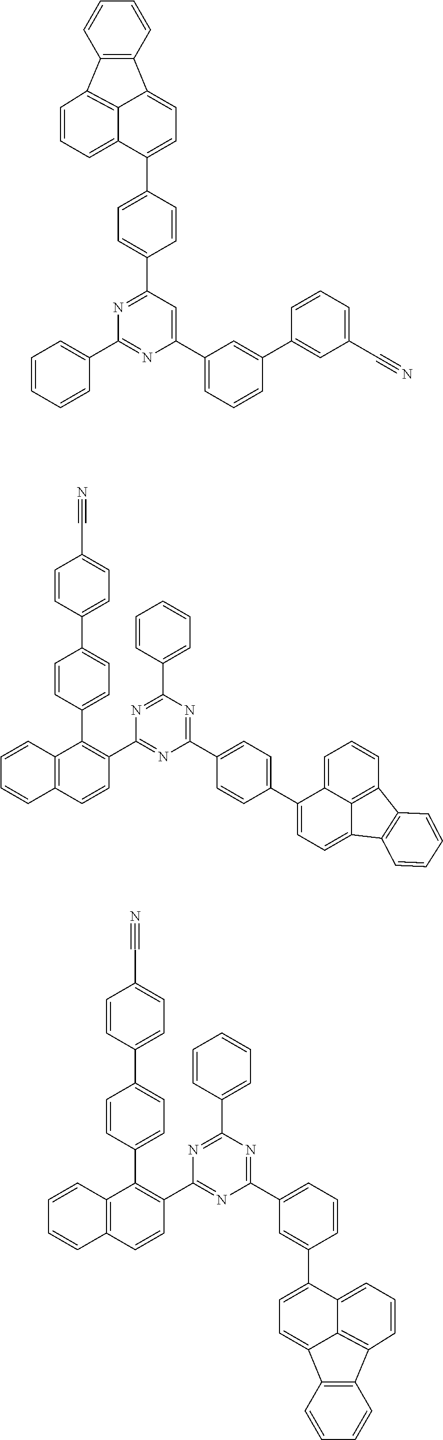 [SMILES: N#Cc1cccc(-c2cccc(-c3cc(-c4ccc(-c5ccc6c7c(cccc57)-c5ccccc5-6)cc4)nc(-c4ccccc4)n3)c2)c1 ; N#Cc1ccc(-c2ccc(-c3c(-c4nc(-c5ccccc5)nc(-c5ccc(-c6ccc7c8c(cccc68)-c6ccccc6-7)cc5)n4)ccc4ccccc34)cc2)cc1 ; N#Cc1ccc(-c2ccc(-c3c(-c4nc(-c5ccccc5)nc(-c5cccc(-c6ccc7c8c(cccc68)-c6ccccc6-7)c5)n4)ccc4ccccc34)cc2)cc1]

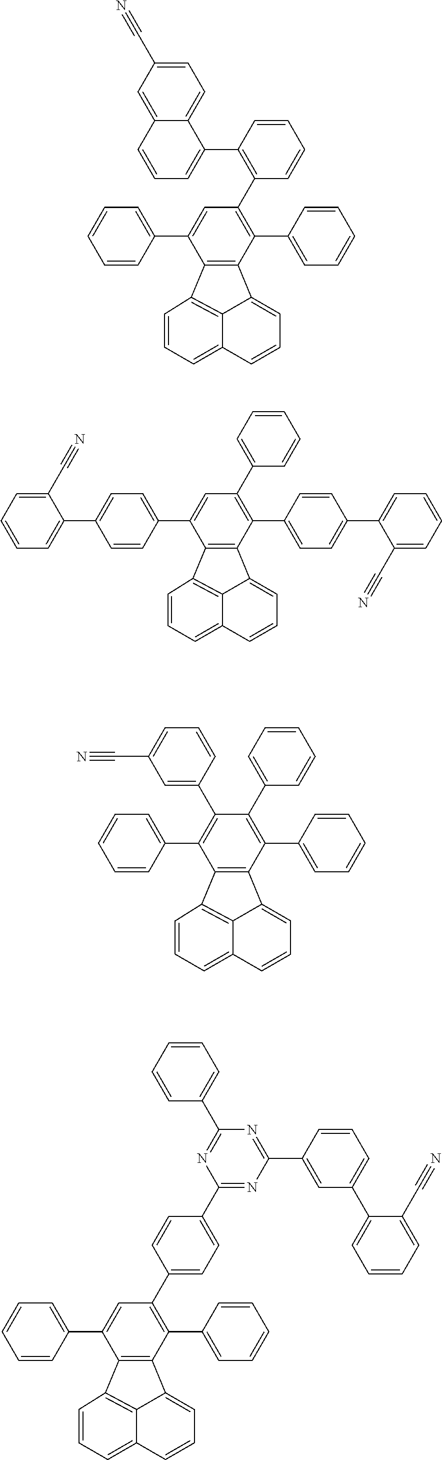 [SMILES: N#Cc1ccc2c(-c3ccccc3-c3cc(-c4ccccc4)c4c(c3-c3ccccc3)-c3cccc5cccc-4c35)cccc2c1 ; N#Cc1ccccc1-c1ccc(-c2cc(-c3ccccc3)c(-c3ccc(-c4ccccc4C#N)cc3)c3c2-c2cccc4cccc-3c24)cc1 ; N#Cc1cccc(-c2c(-c3ccccc3)c(-c3ccccc3)c3c(c2-c2ccccc2)-c2cccc4cccc-3c24)c1 ; N#Cc1ccccc1-c1cccc(-c2nc(-c3ccccc3)nc(-c3ccc(-c4cc(-c5ccccc5)c5c(c4-c4ccccc4)-c4cccc6cccc-5c46)cc3)n2)c1]

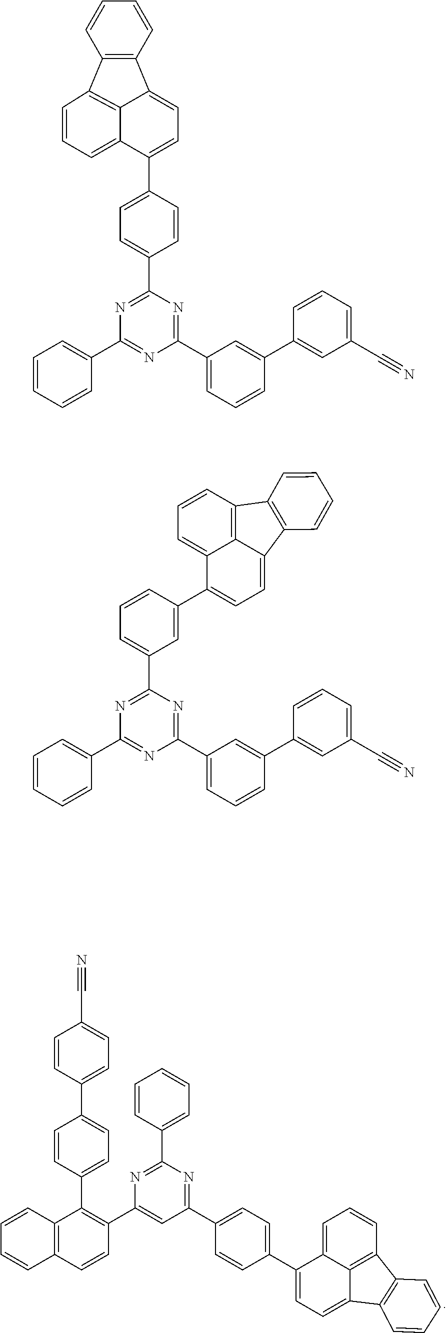 [SMILES: N#Cc1cccc(-c2cccc(-c3nc(-c4ccccc4)nc(-c4ccc(-c5ccc6c7c(cccc57)-c5ccccc5-6)cc4)n3)c2)c1 ; N#Cc1cccc(-c2cccc(-c3nc(-c4ccccc4)nc(-c4cccc(-c5ccc6c7c(cccc57)-c5ccccc5-6)c4)n3)c2)c1 ; N#Cc1ccc(-c2ccc(-c3c(-c4cc(-c5ccc(-c6ccc7c8c(cccc68)-c6ccccc6-7)cc5)nc(-c5ccccc5)n4)ccc4ccccc34)cc2)cc1]

One embodiment of the present specification provides an organic light emitting device including the compound of Chemical Formula 1.

In the present specification, a description of a certain member being placed “o” another member includes not only a case of the one member being in contact with the another member but a case of still another member being present between the two members.

In the present specification, a description of a certain part “including” certain constituents means capable of further including other constituents, and does not exclude other constituents unless particularly stated on the contrary.

In the present specification, the “layer” has a meaning compatible with a ‘film’ mainly used in the art, and means coating covering a target area. The size of the “layer” is not limited, and each “layer” can have the same or a different size. According to one embodiment, the size of the “layer” can be the same as the whole device, can correspond to the size of a specific functional area, or can be as small as a single sub-pixel.

In the present specification, a meaning of a specific A material being included in a B layer includes both i) one or more types of A materials being included in one B layer, and ii) a B layer being formed in one or more layers, and an A material being included in one or more of the B layers that is a multilayer.

In the present specification, a meaning of a specific A material being included in a C layer or a D layer includes both i) being included in one or more layers of one or more C layers, ii) being included in one or more layers of one or more D layers, or iii) being included in each of one or more C layers and one or more D layers.

One embodiment of the present specification provides an organic light emitting device including a first electrode; a second electrode provided opposite to the first electrode; and one or more organic material layers provided between the first electrode and the second electrode, wherein one or more layers of the organic material layers include the compound of Chemical Formula 1.

The organic material layer of the organic light emitting device of the present specification can be formed in a single layer structure, but can also be formed in a multilayer structure in which two or more organic material layers are laminated. For example, the organic light emitting device of the present specification can have a structure including a hole injection layer, a hole transfer layer, a light emitting layer, an electron transfer layer, an electron injection layer, an electron blocking layer, a hole blocking layer and the like. However, the structure of the organic light emitting device is not limited thereto, and can include a smaller number of organic layers.

According to one embodiment of the present specification, the organic material layer includes an electron injection layer, an electron transfer layer, or an electron injection and transfer layer, and the electron injection layer, the electron transfer layer, or the electron injection and transfer layer includes the compound.

According to one embodiment of the present specification, the organic material layer includes a hole blocking layer, and the hole blocking layer includes the compound.

According to one embodiment of the present specification, the organic material layer includes a light emitting layer.

According to one embodiment of the present specification, the organic material layer includes a hole injection layer, a hole transfer layer, or a hole injection and transfer layer.

According to one embodiment of the present specification, the organic material layer includes an electron blocking layer.

According to one embodiment of the present specification, the organic material layer includes a hole blocking layer.

According to one embodiment of the present specification, the organic light emitting device further includes one, two or more layers selected from the group consisting of a hole injection layer, a hole transfer layer, a hole injection and transfer layer, a light emitting layer, an electron transfer layer, an electron injection layer, an electron injection and transfer layer, a hole blocking layer and an electron blocking layer.

According to one embodiment of the present specification, the organic light emitting device includes a first electrode; a second electrode provided opposite to the first electrode; a light emitting layer provided between the first electrode and the second electrode; and two or more organic material layers provided either between the light emitting layer and the first electrode or between the light emitting layer and the second electrode.

According to one embodiment of the present specification, as the two or more organic material layers, two or more can be selected from the group consisting of a hole injection layer, a hole transfer layer, a hole injection and transfer layer, a light emitting layer, an electron transfer layer, an electron injection layer, an electron injection and transfer layer, a hole blocking layer and an electron blocking layer.

According to one embodiment of the present specification, two or more hole transfer layers are included between the light emitting layer and the first electrode. The two or more hole transfer layers can include materials the same as or different from each other.

According to one embodiment of the present specification, the first electrode is an anode or a cathode.

According to one embodiment of the present specification, the second electrode is a cathode or an anode.

According to one embodiment of the present specification, the organic light emitting device can be an organic light emitting device having a structure in which an anode, one or more organic material layers and a cathode are consecutively laminated on a substrate (normal type).

According to one embodiment of the present specification, the organic light emitting device can be an organic light emitting device having a structure in a reverse direction in which a cathode, one or more organic material layers and an anode are consecutively laminated on a substrate (inverted type).

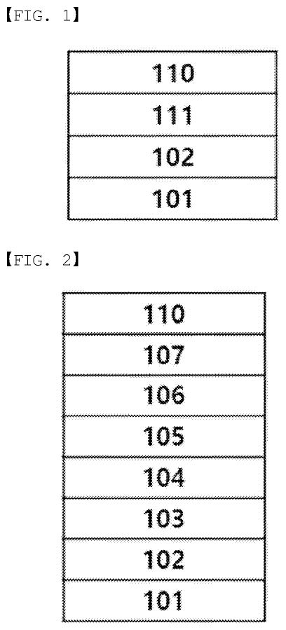

For example, structures of the organic light emitting device according to one embodiment of the present specification are illustrated in FIG. 1 and FIG. 2. FIG. 1 and FIG. 2 only illustrate the organic light emitting device, and the organic light emitting device is not limited thereto.

FIG. 1 illustrates a structure of the organic light emitting device in which a first electrode (102), an organic material layer (111) and a second electrode (110) are consecutively laminated on a substrate (101). The compound of Chemical Formula 1 is included in the organic material layer.

FIG. 2 illustrates a structure of the organic light emitting device in which a first electrode (102), a hole injection layer (103), a first hole transfer layer (104), a second hole transfer layer (105), a light emitting layer (106), an electron injection and transfer layer (107) and a second electrode (110) are consecutively laminated on a substrate (101). The compound of Chemical Formula 1 is included in the electron injection and transfer layer.

The organic light emitting device of the present specification can be manufactured using materials and methods known in the art, except that the electron injection layer, the electron transfer layer, the electron injection and transfer layer, or the hole blocking layer includes the compound, that is, the compound of Chemical Formula 1.

When the organic light emitting device includes a plurality of organic material layers, the organic material layers can be formed with the same materials or different materials.

For example, the organic light emitting device of the present specification can be manufactured by consecutively laminating a first electrode, an organic material layer, and a second electrode on a substrate. Herein, the organic light emitting device can be manufactured by forming an anode on a substrate by depositing a metal, a metal oxide having conductivity, or an alloy thereof using a physical vapor deposition (PVD) method such as sputtering or e-beam evaporation, and forming an organic material layer including a hole injection layer, a hole transfer layer, a light emitting layer and an electron transfer layer thereon, and then depositing a material usable as a cathode thereon. In addition to such a method, the organic light emitting device can also be manufactured by consecutively depositing a cathode material, an organic material layer and an anode material on a substrate.

In addition, the compound of Chemical Formula 1 can be formed into an organic material layer using a solution coating method as well as a vacuum deposition method when manufacturing the organic light emitting device. Herein, the solution coating method means spin coating, dip coating, doctor blading, inkjet printing, screen printing, a spray method, roll coating and the like, but is not limited thereto.

In addition to such a method, the organic light emitting device can also be manufactured by consecutively laminating a cathode material, an organic material layer, and an anode material on a substrate. However, the manufacturing method is not limited thereto.

As the anode material, materials having large work function are normally preferred so that hole injection to an organic material layer is smooth. Examples thereof include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, but are not limited thereto.

As the cathode material, materials having small work function are normally preferred so that electron injection to an organic material layer is smooth. Examples thereof include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

The light emitting layer can include a host material and a dopant material. The host material includes fused aromatic ring derivatives, heteroring-containing compounds or the like. Specifically, the fused aromatic ring derivative includes anthracene derivatives, pyrene derivatives, naphthalene derivatives, pentacene derivatives, phenanthrene compounds, fluoranthene compounds and the like, and the heteroring-containing compound includes dibenzofuran derivatives, ladder-type furan compounds, pyrimidine derivatives and the like, however, the material is not limited thereto.

According to one embodiment of the present specification, the host includes a compound of the following Chemical Formula H-1, but is not limited thereto:

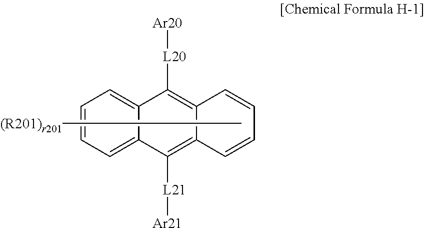

wherein in Chemical Formula H-1:

L20 and L21 are the same as or different from each other, and each independently is a direct bond, a substituted or unsubstituted arylene group, or a substituted or unsubstituted heterocyclic group;

Ar20 and Ar21 are the same as or different from each other, and each independently is hydrogen, deuterium, a substituted or unsubstituted aryl group, or a substituted or unsubstituted heterocyclic group;

R201 is hydrogen, deuterium, a halogen group, a substituted or unsubstituted alkyl group, a substituted or unsubstituted cycloalkyl group, a substituted or unsubstituted aryl group, or a substituted or unsubstituted heterocyclic group; and

r201 is an integer of 1 to 8, and when r201 is 2 or greater, the two or more R201s are the same as or different from each other.

In one embodiment of the present specification, L20 and L21 are the same as or different from each other, and each independently is a direct bond, a monocyclic or polycyclic arylene group having 6 to 30 carbon atoms, or a monocyclic or polycyclic heteroarylene group having 2 to 30 carbon atoms.

In one embodiment of the present specification, L20 and L21 are the same as or different from each other, and each independently is a direct bond; a phenylene group that is unsubstituted or substituted with deuterium; a biphenylylene group that is unsubstituted or substituted with deuterium; a naphthylene group that is unsubstituted or substituted with deuterium; a divalent dibenzofuran group; or a divalent dibenzothiophene group.

In one embodiment of the present specification, Ar20 and Ar21 are the same as or different from each other, and each independently is a substituted or unsubstituted monocyclic or polycyclic aryl group having 6 to 30 carbon atoms, or a substituted or unsubstituted monocyclic or polycyclic heterocyclic group having 2 to 30 carbon atoms.

In one embodiment of the present specification, Ar20 and Ar21 are the same as or different from each other, and each independently is a substituted or unsubstituted monocyclic to tetracyclic aryl group having 6 to 20 carbon atoms, or a substituted or unsubstituted monocyclic to tetracyclic heterocyclic group having 6 to 20 carbon atoms.

In one embodiment of the present specification, Ar20 and Ar21 are the same as or different from each other, and each independently is a phenyl group that is unsubstituted or substituted with deuterium or a monocyclic or polycyclic aryl group having 6 to 20 carbon atoms; a biphenyl group that is unsubstituted or substituted with deuterium or a monocyclic or polycyclic aryl group having 6 to 20 carbon atoms; a naphthyl group that is unsubstituted or substituted with a monocyclic or polycyclic aryl group having 6 to 20 carbon atoms; a dibenzofuran group that is unsubstituted or substituted with a monocyclic or polycyclic aryl group having 6 to 20 carbon atoms; a naphthobenzofuran group that is unsubstituted or substituted with a monocyclic or polycyclic aryl group having 6 to 20 carbon atoms; a dibenzothiophene group that is unsubstituted or substituted with a monocyclic or polycyclic aryl group having 6 to 20 carbon atoms; or a naphthobenzothiophene group that is unsubstituted or substituted with a monocyclic or polycyclic aryl group having 6 to 20 carbon atoms.

In one embodiment of the present specification, Ar20 and Ar21 are the same as or different from each other, and each independently is a phenyl group that is unsubstituted or substituted with deuterium; a biphenyl group that is unsubstituted or substituted with deuterium; a terphenyl group; a naphthyl group that is unsubstituted or substituted with deuterium; a phenanthrene group; a dibenzofuran group; a naphthobenzofuran group; a dibenzothiophene group; or a naphthobenzothiophene group.

In one embodiment of the present specification, Ar20 is a substituted or unsubstituted heterocyclic group, and Ar21 is a substituted or unsubstituted aryl group.

According to one embodiment of the present specification, R201 is hydrogen.

According to one embodiment of the present specification, Chemical Formula H-1 is of the following compound:

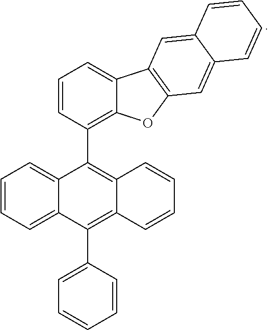 [SMILES: c1ccc(-c2c3ccccc3c(-c3cccc4c3oc3cc5ccccc5cc34)c3ccccc23)cc1]

The dopant material includes aromatic amine derivatives, styrylamine compounds, boron complexes, fluoranthene compounds, metal complexes and the like. Specifically, the aromatic amine derivative is a fused aromatic ring derivative having a substituted or unsubstituted arylamine group and includes arylamine group-including pyrene, anthracene, chrysene, peryflanthene and the like. In addition, the styrylamine compound is a compound in which substituted or unsubstituted arylamine is substituted with at least one arylvinyl group, and one, two or more substituents selected from the group consisting of an aryl group, a silyl group, an alkyl group, a cycloalkyl group and an arylamine group are substituted or unsubstituted. Specifically, styrylamine, styryldiamine, styryltriamine, styryltetramine or the like is included, however, the styrylamine compound is not limited thereto. In addition, the metal complex includes iridium complexes, platinum complexes or the like, but is not limited thereto.

According to one embodiment of the present specification, the dopant includes a compound of the following Chemical Formula D-1, but is not limited thereto:

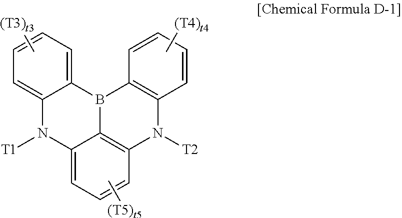

wherein in Chemical Formula D-1:

T1 to T5 are the same as or different from each other, and each independently is hydrogen, a substituted or unsubstituted alkyl group, a substituted or unsubstituted amine group, or a substituted or unsubstituted aryl group;

t3 and t4 are each an integer of 1 to 4;

t5 is an integer of 1 to 3;

when t3 is 2 or greater, the two or more T3s are the same as or different from each other;

when t4 is 2 or greater, the two or more T4s are the same as or different from each other; and

when t5 is 2 or greater, the two or more T5s are the same as or different from each other.

According to one embodiment of the present specification, T1 to T5 are the same as or different from each other, and each independently is hydrogen, a substituted or unsubstituted linear or branched alkyl group having 1 to 30 carbon atoms, a substituted or unsubstituted monocyclic or polycyclic arylamine group having 6 to 30 carbon atoms, or a substituted or unsubstituted monocyclic or polycyclic aryl group having 6 to 30 carbon atoms.

According to one embodiment of the present specification, T1 to T5 are the same as or different from each other, and each independently is hydrogen; a linear or branched alkyl group having 1 to 30 carbon atoms; a monocyclic or polycyclic arylamine group having 6 to 30 carbon atoms; or a monocyclic or polycyclic aryl group having 6 to 30 carbon atoms that is unsubstituted or substituted with a linear or branched alkyl group having 1 to 30 carbon atoms.

According to one embodiment of the present specification, T1 to T5 are the same as or different from each other, and each independently is hydrogen; a methyl group; a tert-butyl group; or a phenyl group that is unsubstituted or substituted with a tert-butyl group.

According to one embodiment of the present specification, Chemical Formula D-1 is the following compound:

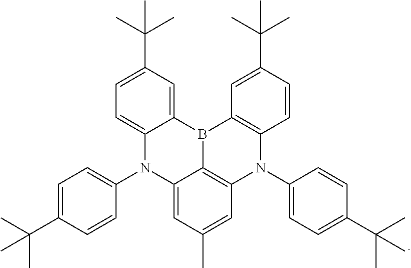 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

The hole injection layer is a layer receiving holes from an electrode. The hole injection material preferably has, by having an ability to transfer holes, a hole receiving effect from an anode and an excellent hole injection effect for a light emitting layer or a light emitting material. In addition, the hole injection material is preferably a material having an excellent ability to prevent excitons generated in the light emitting layer from moving to an electron injection layer or an electron injection material. In addition, a material having an excellent thin film forming ability is preferred. In addition, the highest occupied molecular orbital (HOMO) of the hole injection material is preferably in between the work function of an anode material and the HOMO of surrounding organic material layers. Specific examples of the hole injection material include metal porphyrins, oligothiophene, arylamine-based organic materials; hexanitrile hexaazatriphenylene-based organic materials; quinacridone-based organic materials; perylene-based organic materials; polythiophene-based conductive polymers such as anthraquinone or polyaniline, and the like, but are not limited thereto.

According to one embodiment of the present specification, the hole injection layer includes a compound of the following Chemical Formula HI-1, but is not limited thereto:

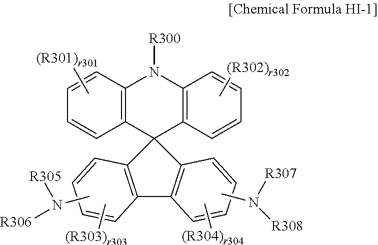

wherein in Chemical Formula HI-1:

R301 to R308 are the same as or different from each other, and each independently is hydrogen, deuterium, a cyano group, a substituted or unsubstituted alkyl group, a substituted or unsubstituted amine group, a substituted or unsubstituted aryl group, or a substituted or unsubstituted heteroaryl group, or bond to adjacent groups to form a substituted or unsubstituted ring;

r301 and r302 are each an integer of 1 to 4;

r303 and r304 are each an integer of 1 to 3;

when r301 is 2 or greater, R301s are the same as or different from each other;

when r302 is 2 or greater, R302s are the same as or different from each other;

when r303 is 2 or greater, R303s are the same as or different from each other; and

when r304 is 2 or greater, R304s are the same as or different from each other.

According to one embodiment of the present specification, R301 to R304 are hydrogen.

According to one embodiment of the present specification, R300 is a substituted or unsubstituted aryl group.

According to one embodiment of the present specification, R300 is a monocyclic or polycyclic aryl group having 6 to 30 carbon atoms.

According to one embodiment of the present specification, R300 is a phenyl group.

According to one embodiment of the present specification, R305 to R308 are the same as or different from each other, and each independently is a substituted or unsubstituted aryl group or a substituted or unsubstituted heteroaryl group.

According to one embodiment of the present specification, R305 to R308 are the same as or different from each other, and each independently is a monocyclic or polycyclic aryl group having 6 to 30 carbon atoms; or a monocyclic or polycyclic heteroaryl group having 2 to 30 carbon atoms that is unsubstituted or substituted with a monocyclic or polycyclic aryl group having 6 to 30 carbon atoms.

According to one embodiment of the present specification, R305 to R308 are the same as or different from each other, and each independently is a phenyl group; or a carbazole group that is unsubstituted or substituted with a phenyl group.

According to one embodiment of the present specification, Chemical Formula HI-1 is the following compound:

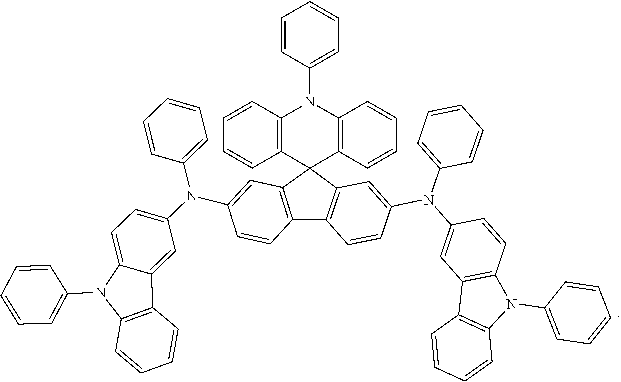 [SMILES: c1ccc(N(c2ccc3c(c2)C2(c4cc(N(c5ccccc5)c5ccc6c(c5)c5ccccc5n6-c5ccccc5)ccc4-3)c3ccccc3N(c3ccccc3)c3ccccc32)c2ccc3c(c2)c2ccccc2n3-c2ccccc2)cc1]

The hole transfer layer is a layer receiving holes from a hole injection layer and transferring the holes to a light emitting layer. As the hole transfer material, materials having, as a material capable of receiving holes from an anode or a hole injection layer and moving the holes to a light emitting layer, high mobility for the holes are preferred. Specific examples thereof include arylamine-based organic materials, conductive polymers, block copolymers having conjugated parts and non-conjugated parts together, and the like, but are not limited thereto.

According to one embodiment of the present specification, the hole transfer layer includes a compound of the following Chemical Formula HT-1, but is not limited thereto:

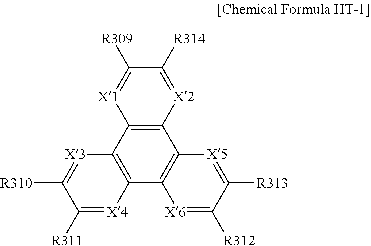

wherein in Chemical Formula HT-1:

at least one of X′1 to X′6 is N, and the rest are CH; and

R309 to R313 are the same as or different from each other, and each independently is hydrogen, deuterium, a cyano group, a substituted or unsubstituted alkyl group, a substituted or unsubstituted amine group, a substituted or unsubstituted aryl group, or a substituted or unsubstituted heteroaryl group, or bond to adjacent groups to form a substituted or unsubstituted ring.

According to one embodiment of the present specification, X′1 to X′6 are N.

According to one embodiment of the present specification, R309 to R313 are a cyano group.

According to one embodiment of the present specification, Chemical Formula HT-1 is the following compound:

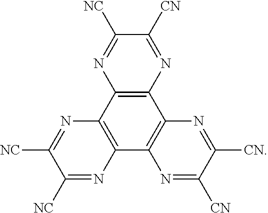 [SMILES: N#Cc1nc2c3nc(C#N)c(C#N)nc3c3nc(C#N)c(C#N)nc3c2nc1C#N]

According to one embodiment of the present specification, the hole transfer layer includes a compound of the following Chemical Formula HT-2, but is not limited thereto:

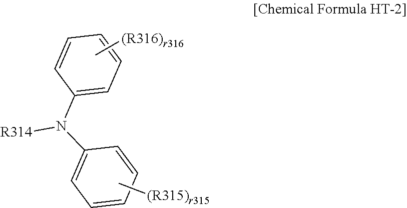

wherein in Chemical Formula HT-2:

R314 to R316 are the same as or different from each other, and each independently is any one selected from the group consisting of hydrogen, deuterium; a substituted or unsubstituted alkyl group, a substituted or unsubstituted aryl group, a substituted or unsubstituted heteroaryl group, and combinations thereof, or bond to adjacent groups to form a substituted or unsubstituted ring;

r315 is an integer of 1 to 4, and when r315 is 2 or greater, the two or more R315s are the same as or different from each other; and

r316 is an integer of 1 to 4, and when r316 is 2 or greater, the two or more R316s are the same as or different from each other.

According to one embodiment of the present specification, R314 is any one selected from the group consisting of a substituted or unsubstituted aryl group, a substituted or unsubstituted heteroaryl group, and combinations thereof.

According to one embodiment of the present specification, R314 is any one selected from the group consisting of a carbazole group, a phenyl group, a biphenyl group, and combinations thereof.

According to one embodiment of the present specification, R315 and R316 are the same as or different from each other, and each independently is a substituted or unsubstituted aryl group.

According to one embodiment of the present specification, R315 and R316 are a phenyl group.

According to one embodiment of the present specification, Chemical Formula HT-2 is the following compound:

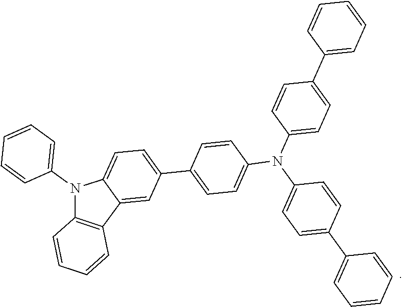 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

The electron transfer layer is a layer receiving electrons from an electron injection layer and transferring the electrons to a light emitting layer. When the organic light emitting device according to one embodiment of the present specification includes an additional electron transfer layer other than the electron transfer layer including Chemical Formula 1, materials capable of favorably receiving electrons from a cathode, moving the electrons to a light emitting layer, and having high mobility for the electrons are preferred as the electron transfer material. Specific examples thereof include Al complexes of 8-hydroxyquinoline; complexes including Alq₃; organic radical compounds; hydroxyflavon-metal complexes and the like, but are not limited thereto. The electron transfer layer can be used together with any desired cathode material as used in the art. Particularly, the suitable cathode material is a common material having low work function and having an aluminum layer or a silver layer following. Specifically, cesium, barium, calcium, ytterbium, samarium and the like are included, and in each case, an aluminum layer or a silver layer follows.

The electron injection layer is a layer receiving electrons from an electrode. When the organic light emitting device according to one embodiment of the present specification includes an additional electron injection other than the electron injection layer including Chemical Formula 1, materials having an excellent electron transferring ability, having an electron receiving effect from a second electrode, and having an excellent electron injection effect for a light emitting layer or light emitting material are preferred as the electron injection material. In addition, materials preventing excitons generated in the light emitting layer from moving to a hole injection layer, and having an excellent thin film forming ability are preferred. Specific examples thereof include fluorenone, anthraquinodimethane, diphenoquinone, thiopyran dioxide, oxazole, oxadiazole, triazole, imidazole, perylene tetracarboxylic acid, fluorenylidene methane, anthrone or the like, and derivatives thereof, metal complex compounds, nitrogen-containing 5-membered ring derivatives, and the like, but are not limited thereto.

The metal complex compound includes 8-hydroxyquinolinato lithium, bis(8-hydroxyquinolinato)zinc, bis(8-hydroxyquinolinato)copper, bis(8-hydroxyquinolinato)-manganese, tris(8-hydroxyquinolinato)aluminum, tris(2-methyl-8-hydroxyquinolinato)aluminum, tris(8-hydroxy-quinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium and the like, but is not limited thereto.

The electron blocking layer is a layer capable of enhancing lifetime and efficiency of a device by preventing electrons injected from an electron injection layer from passing through a light emitting layer and entering a hole injection layer. Known material can be used without limit, and the electron blocking layer can be formed between the light emitting layer and the hole injection layer, or between the light emitting layer and a layer carrying out hole injection and hole transfer at the same time.

The hole blocking layer is a layer blocking holes from reaching a cathode, and can be generally formed under the same condition as the electron injection layer. When the organic light emitting device according to one embodiment of the present specification includes an additional hole blocking layer other than the hole blocking layer including Chemical Formula 1, specific examples thereof can include oxadiazole derivatives, triazole derivatives, phenanthroline derivatives, aluminum complexes and the like, but are not limited thereto.

The organic light emitting device according to the present specification can be a top-emission type, a bottom-emission type or a dual-emission type depending on the materials used.

The organic light emitting device according to the present specification can be included in various electronic devices and used. For example, the electronic device can be a display panel, a touch panel, a solar module, an illumination device or the like, but is not limited thereto.

### EXAMPLES

Hereinafter, the present specification will be described in detail with reference to examples, comparative examples and the like. However, the examples and the comparative examples according to the present specification can be modified to various other forms, and the scope of the present specification is not to be construed as being limited to the examples and the comparative examples described below. Examples and comparative examples of the present specification are provided in order to more fully describe the present specification to those having average knowledge in the art.

### Synthesis Example

<Synthesis of Compound 1-1>

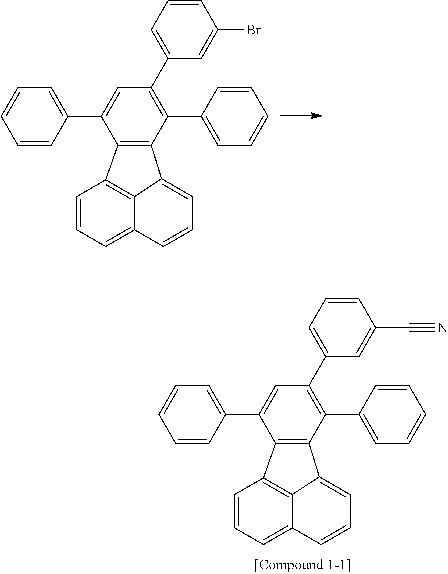 [SMILES: Brc1cccc(-c2cc(-c3ccccc3)c3c(c2-c2ccccc2)-c2cccc4cccc-3c24)c1 ; N#Cc1cccc(-c2cc(-c3ccccc3)c3c(c2-c2ccccc2)-c2cccc4cccc-3c24)c1]

8-(3-Bromophenyl)-7,10-diphenylfluoranthene (20 g, 39.36 mmol) was introduced to DMAc (N,N-dimethylacetamide) (200 mL), and the temperature was raised. When starting to reflux, tetrakis(triphenylphosphine)palladium(0) (1.36 g, 1.18 mmol) was introduced thereto, the result was stirred for 30 minutes, then Zn(CN)₂ (2.3 g, 19.68 mmol) was introduced thereto, and the result was further stirred for 1 hour. After terminating the reaction, the result was cooled, and ethanol slurry purified to prepare [Compound 1-1] (15 g, yield 84%).

[M+H]⁺=456

<Synthesis of Compound 1-2>

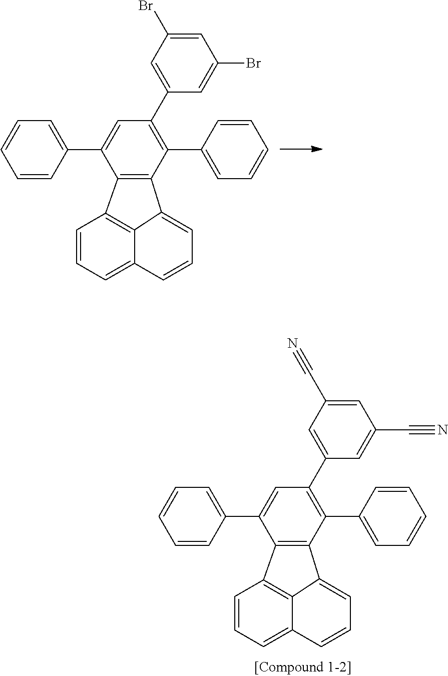 [SMILES: Brc1cc(Br)cc(-c2cc(-c3ccccc3)c3c(c2-c2ccccc2)-c2cccc4cccc-3c24)c1 ; N#Cc1cc(C#N)cc(-c2cc(-c3ccccc3)c3c(c2-c2ccccc2)-c2cccc4cccc-3c24)c1]

[Compound 1-2] was prepared in the same manner as in Synthesis of [Compound 1-1] except that 8-(3,5-dibromophenyl)-7,10-diphenylfluoranthene was used instead of 8-(3-bromophenyl)-7,10-diphenylfluoranthene.

[M+H]⁺=481

<Synthesis of Compound 1-3>

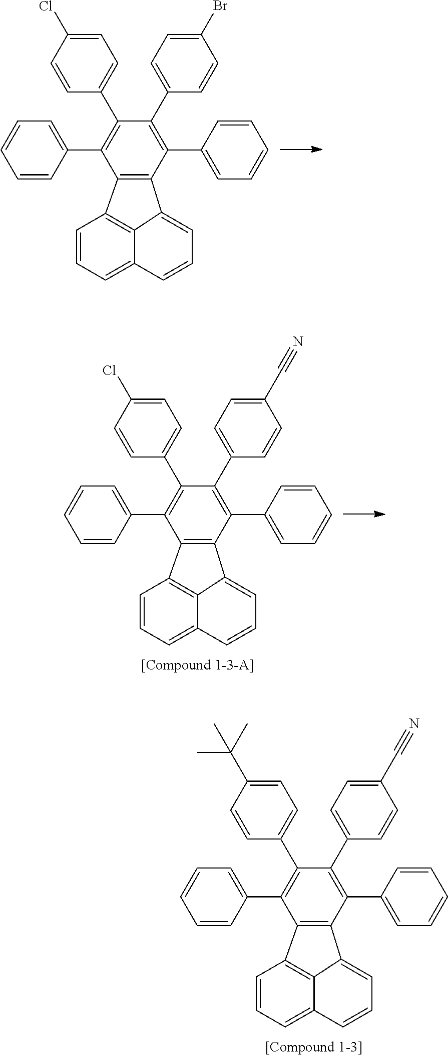 [SMILES: Clc1ccc(-c2c(-c3ccc(Br)cc3)c(-c3ccccc3)c3c(c2-c2ccccc2)-c2cccc4cccc-3c24)cc1 ; N#Cc1ccc(-c2c(-c3ccc(Cl)cc3)c(-c3ccccc3)c3c(c2-c2ccccc2)-c2cccc4cccc-3c24)cc1 ; CC(C)(C)c1ccc(-c2c(-c3ccc(C#N)cc3)c(-c3ccccc3)c3c(c2-c2ccccc2)-c2cccc4cccc-3c24)cc1]

[Compound 1-3-A] was prepared in the same manner as in Synthesis of [Compound 1-1] except that 8-(4-bromophenyl)-9-(4-chlorophenyl)-7,10-diphenylfluoranthene was used instead of 8-(3-bromophenyl)-7,10-diphenylfluoranthene.

[M+H]⁺=566

[Compound 1-3-A] (20 g, 35.39 mmol) and 2-(tert-butyl)-4,4,5,5-tetramethyl-1,3,2-dioxaborolane (7.4 g, 40.25 mmol) were dissolved in tetrahydrofuran (200 mL), and stirred. An aqueous potassium carbonate (9.8 g, 70.78 mmol) solution was introduced thereto, and the temperature was raised. When starting to reflux, a tetrakis(triphenyl-phosphine)palladium(0) (1.2 g, 1.06 mmol) catalyst was introduced thereto, and the result was stirred for 3 hours. After terminating the reaction, the result was cooled, and ethanol slurry purified to prepare [Compound 1-3] (17 g, yield 82%).

[M+H]⁺=588

<Synthesis of Compound 1-4>

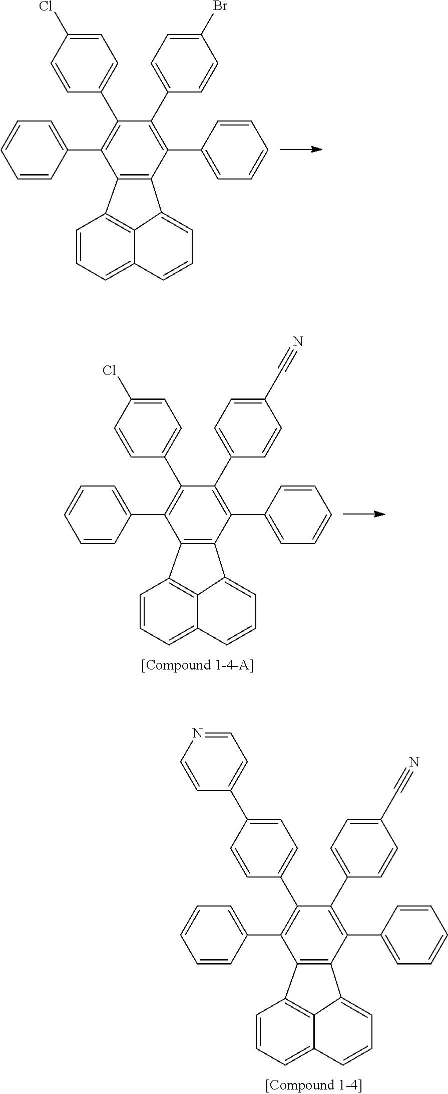 [SMILES: Clc1ccc(-c2c(-c3ccc(Br)cc3)c(-c3ccccc3)c3c(c2-c2ccccc2)-c2cccc4cccc-3c24)cc1 ; N#Cc1ccc(-c2c(-c3ccc(Cl)cc3)c(-c3ccccc3)c3c(c2-c2ccccc2)-c2cccc4cccc-3c24)cc1 ; N#Cc1ccc(-c2c(-c3ccc(-c4ccncc4)cc3)c(-c3ccccc3)c3c(c2-c2ccccc2)-c2cccc4cccc-3c24)cc1]

[Compound 1-4-A] was prepared in the same manner as in Synthesis of [Compound 1-1] except that 8-(4-bromophenyl)-9-(4-chlorophenyl)-7,10-diphenylfluoranthene was used instead of 8-(3-bromophenyl)-7,10-diphenylfluoranthene.

[M+H]⁺=566

[Compound 1-4] was prepared in the same manner as in Synthesis of [Compound 1-3] except that pyridin-4-ylboronic acid was used instead of 2-(tert-butyl)-4,4,5,5-tetramethyl-1,3,2-dioxaborolane.

[M+H]⁺=609

<Synthesis of Compound 1-5>

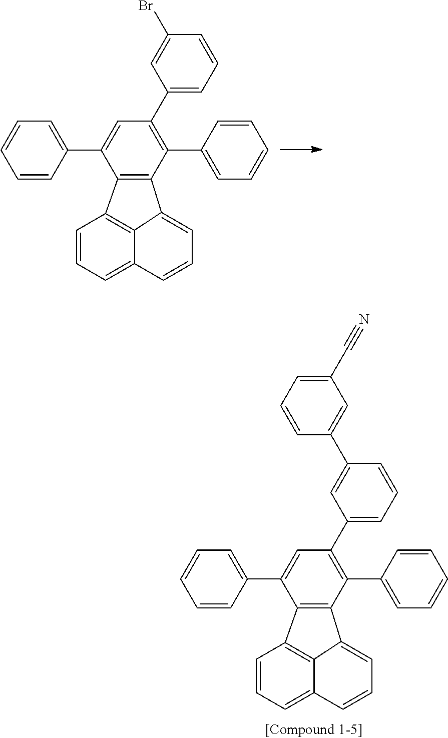 [SMILES: Brc1cccc(-c2cc(-c3ccccc3)c3c(c2-c2ccccc2)-c2cccc4cccc-3c24)c1 ; N#Cc1cccc(-c2cccc(-c3cc(-c4ccccc4)c4c(c3-c3ccccc3)-c3cccc5cccc-4c35)c2)c1]

[Compound 1-5] was prepared in the same manner as in Synthesis of [Compound 1-3] except that 8-(3-bromophenyl)-7,10-diphenylfluoranthene was used instead of [Compound 1-3-A], and (3-cyanophenyl) boronic acid was used instead of 2-(tert-butyl)-4,4,5,5-tetramethyl-1,3,2-dioxaborolane.

[M+H]⁺=532

<Synthesis of Compound 1-6>

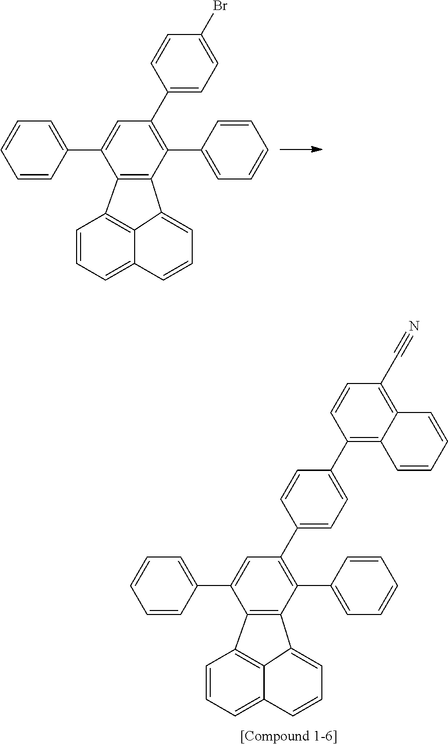 [SMILES: Brc1ccc(-c2cc(-c3ccccc3)c3c(c2-c2ccccc2)-c2cccc4cccc-3c24)cc1 ; N#Cc1ccc(-c2ccc(-c3cc(-c4ccccc4)c4c(c3-c3ccccc3)-c3cccc5cccc-4c35)cc2)c2ccccc12]

[Compound 1-6] was prepared in the same manner as in Synthesis of [Compound 1-5] except that 8-(4-bromophenyl)-7,10-diphenylfluoranthene was used instead of 8-(3-bromophenyl)-7,10-diphenylfluoranthene, and (4-cyanonaphthalen-1-yl)boronic acid was used instead of (3-cyanophenyl)boronic acid.

[M+H]⁺=582

<Synthesis of Compound 1-7>

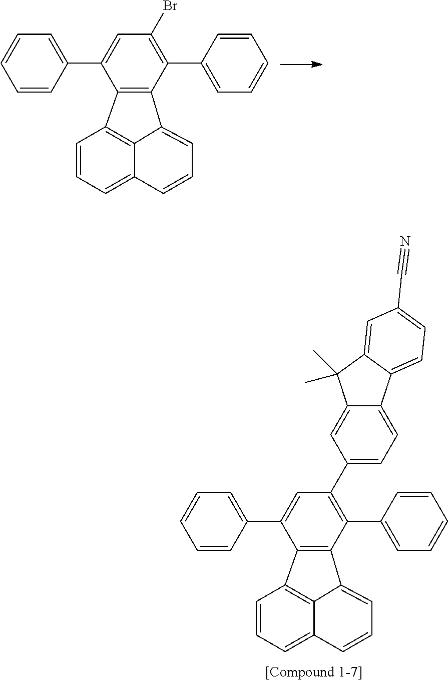 [SMILES: Brc1cc(-c2ccccc2)c2c(c1-c1ccccc1)-c1cccc3cccc-2c13 ; CC1(C)c2cc(C#N)ccc2-c2ccc(-c3cc(-c4ccccc4)c4c(c3-c3ccccc3)-c3cccc5cccc-4c35)cc21]

[Compound 1-7] was prepared in the same manner as in Synthesis of [Compound 1-6] except that 8-bromo-7,10-diphenylfluoranthene was used instead of 8-(4-bromophenyl)-7,10-diphenylfluoranthene, and 9,9-dimethyl-7-(4,4,5,5-tetramethyl-1,3,2-dioxaborolan-2-yl)-9H-fluorene-2-carbonitrile was used instead of (4-cyanonaphthalen-1-yl)boronic acid.

[M+H]⁺=572

<Synthesis of Compound 1-8>

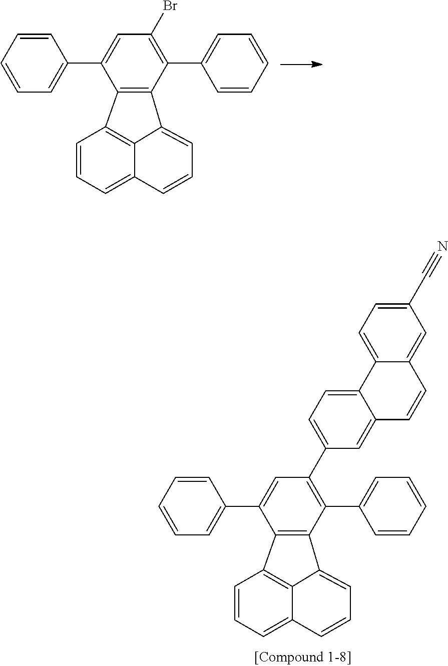 [SMILES: Brc1cc(-c2ccccc2)c2c(c1-c1ccccc1)-c1cccc3cccc-2c13 ; N#Cc1ccc2c(ccc3cc(-c4cc(-c5ccccc5)c5c(c4-c4ccccc4)-c4cccc6cccc-5c46)ccc32)c1]

[Compound 1-8] was prepared in the same manner as in Synthesis of [Compound 1-7] except that (7-cyanophenanthren-2-yl)boronic acid was used instead of 9,9-dimethyl-7-(4,4,5,5-tetramethyl-1,3,2-dioxaborolan-2-yl)-9H-fluorene-2-carbonitrile.

[M+H]⁺=556

<Synthesis of Compound 1-9>

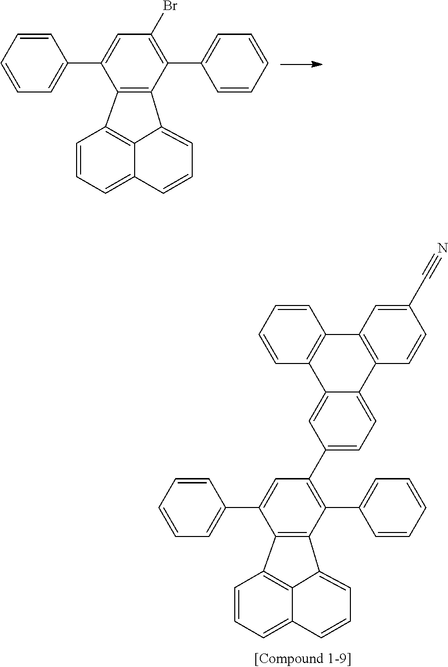 [SMILES: Brc1cc(-c2ccccc2)c2c(c1-c1ccccc1)-c1cccc3cccc-2c13 ; N#Cc1ccc2c3ccc(-c4cc(-c5ccccc5)c5c(c4-c4ccccc4)-c4cccc6cccc-5c46)cc3c3ccccc3c2c1]

[Compound 1-9] was prepared in the same manner as in Synthesis of [Compound 1-7] except that (7-cyanotriphenylen-2-yl)boronic acid was used instead of 9,9-dimethyl-7-(4,4,5,5-tetramethyl-1,3,2-dioxaborolan-2-yl)-9H-fluorene-2-carbonitrile.

[M+H]⁺=556

<Synthesis of Compound 1-10>

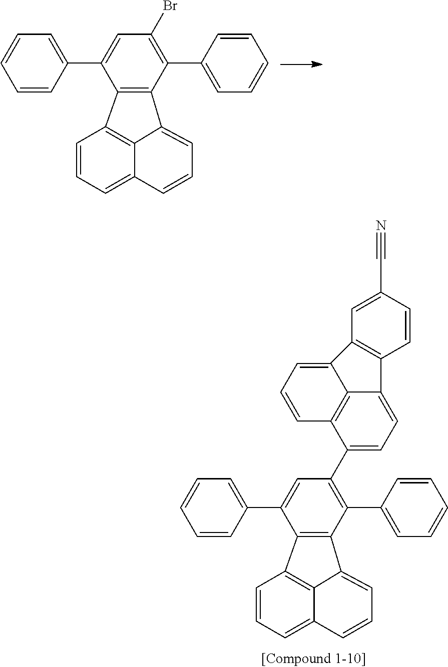 [SMILES: Brc1cc(-c2ccccc2)c2c(c1-c1ccccc1)-c1cccc3cccc-2c13 ; N#Cc1ccc2c(c1)-c1cccc3c(-c4cc(-c5ccccc5)c5c(c4-c4ccccc4)-c4cccc6cccc-5c46)ccc-2c13]

[Compound 1-10] was prepared in the same manner as in Synthesis of [Compound 1-7] except that (8-cyanofluoranthen-3-yl)boronic acid was used instead of 9,9-dimethyl-7-(4,4,5,5-tetramethyl-1,3,2-dioxaborolan-2-yl)-9H-fluorene-2-carbonitrile.

[M+H]⁺=580

<Synthesis of Compound 1-11>

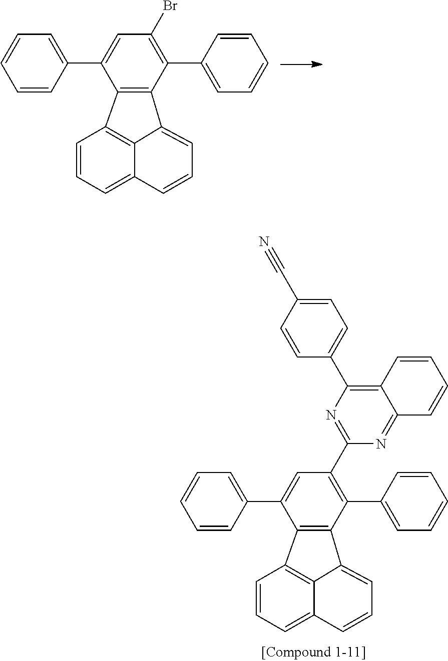 [SMILES: Brc1cc(-c2ccccc2)c2c(c1-c1ccccc1)-c1cccc3cccc-2c13 ; N#Cc1ccc(-c2nc(-c3cc(-c4ccccc4)c4c(c3-c3ccccc3)-c3cccc5cccc-4c35)nc3ccccc23)cc1]

[Compound 1-11] was prepared in the same manner as in Synthesis of [Compound 1-7] except that (4-(4-cyanophenyl)quinazolin-2-yl)boronic acid was used instead of 9,9-dimethyl-7-(4,4,5,5-tetramethyl-1,3,2-dioxaborolan-2-yl)-9H-fluorene-2-carbonitrile.

[M+H]⁺=584

<Synthesis of Compound 1-12>

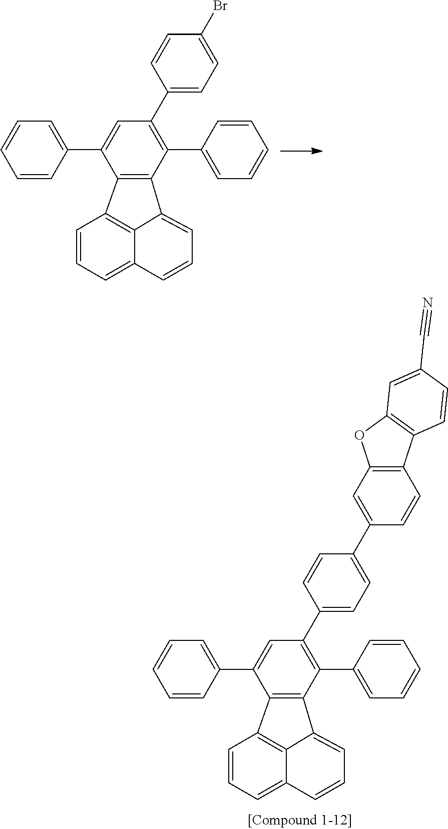 [SMILES: Brc1ccc(-c2cc(-c3ccccc3)c3c(c2-c2ccccc2)-c2cccc4cccc-3c24)cc1 ; N#Cc1ccc2c(c1)oc1cc(-c3ccc(-c4cc(-c5ccccc5)c5c(c4-c4ccccc4)-c4cccc6cccc-5c46)cc3)ccc12]

[Compound 1-12] was prepared in the same manner as in Synthesis of [Compound 1-6] except that (7-cyanodibenzo[b,d]furan-3-yl)boronic acid was used instead of (4-cyanonaphthalen-1-yl)boronic acid.

[M+H]⁺=622

<Synthesis of Compound 1-13>

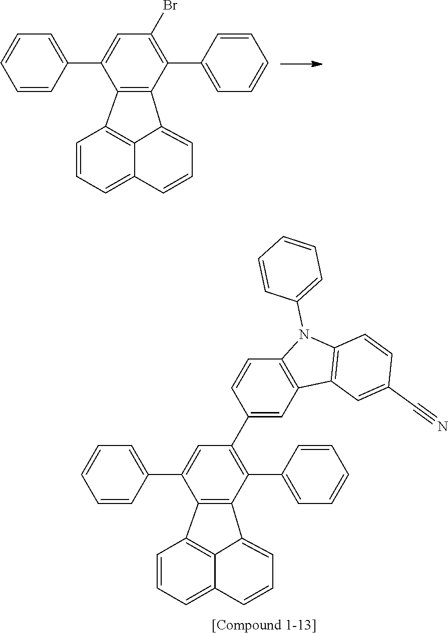 [SMILES: Brc1cc(-c2ccccc2)c2c(c1-c1ccccc1)-c1cccc3cccc-2c13 ; N#Cc1ccc2c(c1)c1cc(-c3cc(-c4ccccc4)c4c(c3-c3ccccc3)-c3cccc5cccc-4c35)ccc1n2-c1ccccc1]

[Compound 1-13] was prepared in the same manner as in Synthesis of [Compound 1-7] except that (6-cyano-9-phenyl-9H-carbazol-3-yl)boronic acid was used instead of 9,9-dimethyl-7-(4,4,5,5-tetramethyl-1,3,2-dioxaborolan-2-yl)-9H-fluorene-2-carbonitrile.

[M+H]⁺=621

<Synthesis of Compound 1-14>

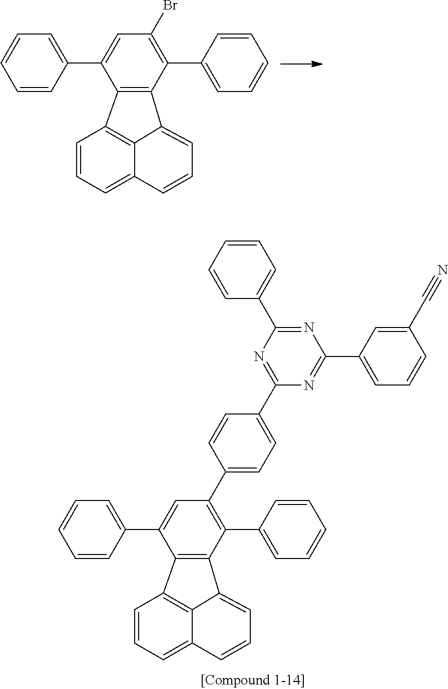 [SMILES: Brc1cc(-c2ccccc2)c2c(c1-c1ccccc1)-c1cccc3cccc-2c13 ; N#Cc1cccc(-c2nc(-c3ccccc3)nc(-c3ccc(-c4cc(-c5ccccc5)c5c(c4-c4ccccc4)-c4cccc6cccc-5c46)cc3)n2)c1]

[Compound 1-14] was prepared in the same manner as in Synthesis of [Compound 1-7] except that 3-(4-phenyl-6-(4-(4,4,5,5-tetramethyl-1,3,2-dioxaborolan-2-yl)phenyl)-1,3,5-triazin-2-yl)benzonitrile was used instead of 9,9-dimethyl-7-(4,4,5,5-tetramethyl-1,3,2-dioxaborolan-2-yl)-9H-fluorene-2-carbonitrile.

[M+H]⁺=687

<Synthesis of Compound 1-15>

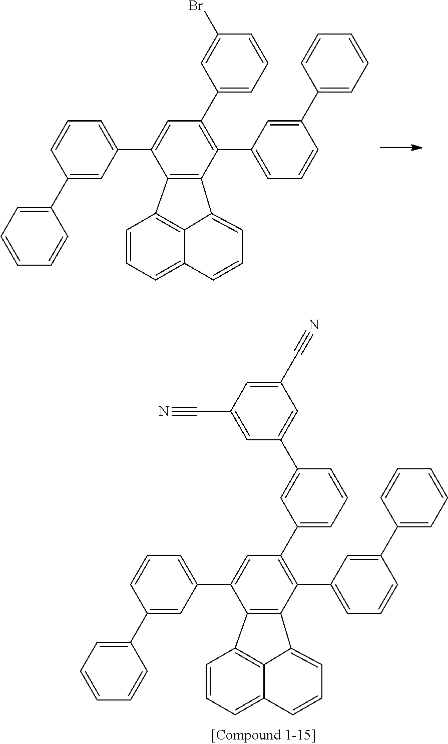 [SMILES: Brc1cccc(-c2cc(-c3cccc(-c4ccccc4)c3)c3c(c2-c2cccc(-c4ccccc4)c2)-c2cccc4cccc-3c24)c1 ; N#Cc1cc(C#N)cc(-c2cccc(-c3cc(-c4cccc(-c5ccccc5)c4)c4c(c3-c3cccc(-c5ccccc5)c3)-c3cccc5cccc-4c35)c2)c1]

[Compound 1-15] was prepared in the same manner as in Synthesis of [Compound 1-5] except that 7,10-di([1,1′-biphenyl]-3-yl)-8-(3-bromophenyl)fluoranthene was used instead of 8-(3-bromophenyl)-7,10-diphenylfluoranthene, and (3,5-dicyanophenyl)boronic acid was used instead of (3-cyanophenyl)boronic acid.

[M+H]⁺=709

<Synthesis of Compound 1-16>

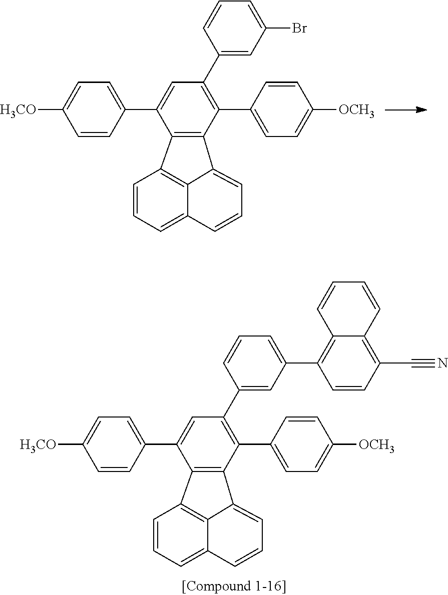 [SMILES: COc1ccc(-c2cc(-c3cccc(Br)c3)c(-c3ccc(OC)cc3)c3c2-c2cccc4cccc-3c24)cc1 ; COc1ccc(-c2cc(-c3cccc(-c4ccc(C#N)c5ccccc45)c3)c(-c3ccc(OC)cc3)c3c2-c2cccc4cccc-3c24)cc1]

[Compound 1-16] was prepared in the same manner as in Synthesis of [Compound 1-5] except that 8-(3-bromophenyl)-7,10-bis(4-methoxyphenyl)fluoranthene was used instead of 8-(3-bromophenyl)-7,10-diphenylfluoranthene, and (4-cyanonaphthalen-1-yl)boronic acid was used instead of (3-cyanophenyl)boronic acid.

[M+H]⁺=642

<Synthesis of Compound 1-17>

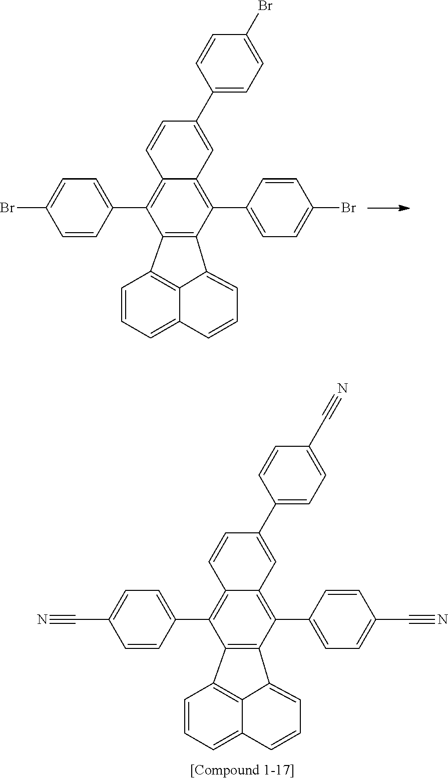 [SMILES: Brc1ccc(-c2ccc3c(-c4ccc(Br)cc4)c4c(c(-c5ccc(Br)cc5)c3c2)-c2cccc3cccc-4c23)cc1 ; N#Cc1ccc(-c2ccc3c(-c4ccc(C#N)cc4)c4c(c(-c5ccc(C#N)cc5)c3c2)-c2cccc3cccc-4c23)cc1]

[Compound 1-17] was prepared in the same manner as in Synthesis of [Compound 1-1] except that 7,9,12-tris(4-bromophenyl)benzo[k]fluoranthene was used instead of 8-(3-bromophenyl)-7,10-diphenylfluoranthene.

[M+H]⁺=556

<Synthesis of Compound 1-18>

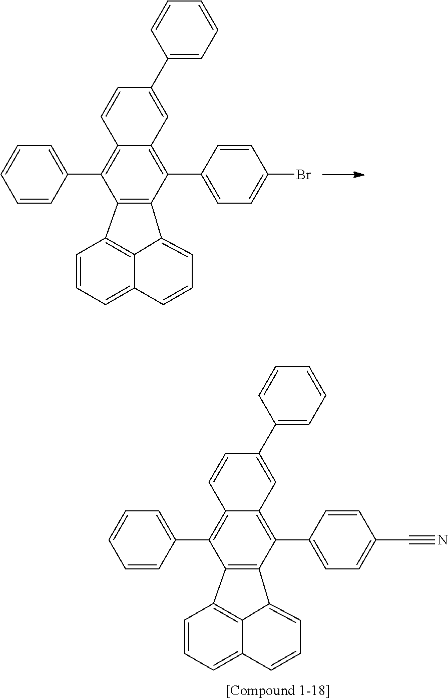 [SMILES: Brc1ccc(-c2c3c(c(-c4ccccc4)c4ccc(-c5ccccc5)cc24)-c2cccc4cccc-3c24)cc1 ; N#Cc1ccc(-c2c3c(c(-c4ccccc4)c4ccc(-c5ccccc5)cc24)-c2cccc4cccc-3c24)cc1]

[Compound 1-18] was prepared in the same manner as in Synthesis of [Compound 1-1] except that 7-(4-bromophenyl)-9,12-diphenylbenzo[k]fluoranthene was used instead of 8-(3-bromophenyl)-7,10-diphenylfluoranthene.

[M+H]⁺=506

<Synthesis of Compound 1-19>

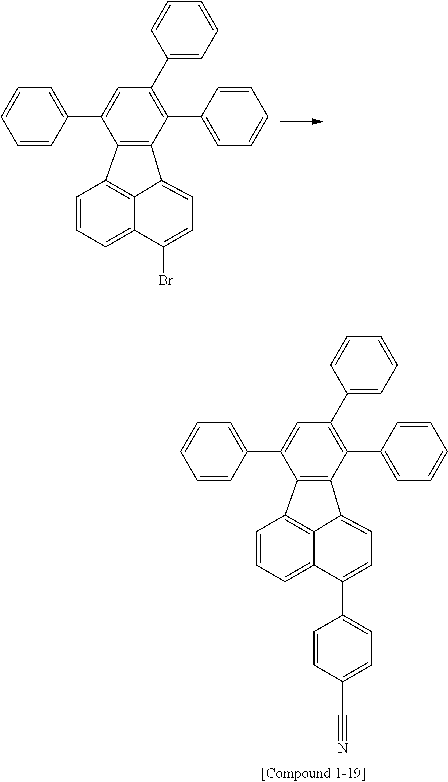 [SMILES: Brc1ccc2c3c(cccc13)-c1c(-c3ccccc3)cc(-c3ccccc3)c(-c3ccccc3)c1-2 ; N#Cc1ccc(-c2ccc3c4c(cccc24)-c2c(-c4ccccc4)cc(-c4ccccc4)c(-c4ccccc4)c2-3)cc1]

[Compound 1-19] was prepared in the same manner as in Synthesis of [Compound 1-5] except that 3-bromo-7,9,10-triphenylfluoranthene was used instead of 8-(3-bromophenyl)-7,10-diphenylfluoranthene, and (4-cyanophenyl)boronic acid was used instead of (3-cyanophenyl)boronic acid.

[M+H]⁺=532

<Synthesis of Compound 1-20>

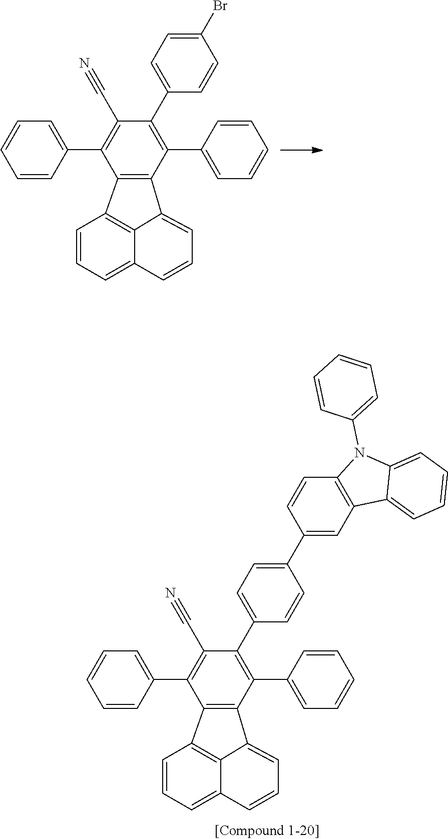 [SMILES: N#Cc1c(-c2ccc(Br)cc2)c(-c2ccccc2)c2c(c1-c1ccccc1)-c1cccc3cccc-2c13 ; N#Cc1c(-c2ccc(-c3ccc4c(c3)c3ccccc3n4-c3ccccc3)cc2)c(-c2ccccc2)c2c(c1-c1ccccc1)-c1cccc3cccc-2c13]

[Compound 1-20] was prepared in the same manner as in Synthesis of [Compound 1-5] except that 9-(4-bromophenyl)-7,10-diphenylfluoranthene-8-carbonitrile was used instead of 8-(3-bromophenyl)-7,10-diphenylfluoranthene, and (9-phenyl-9H-carbazol-3-yl)boronic acid was used instead of (3-cyanophenyl)boronic acid.

[M+H]⁺=697

<Synthesis of Compound 1-21>

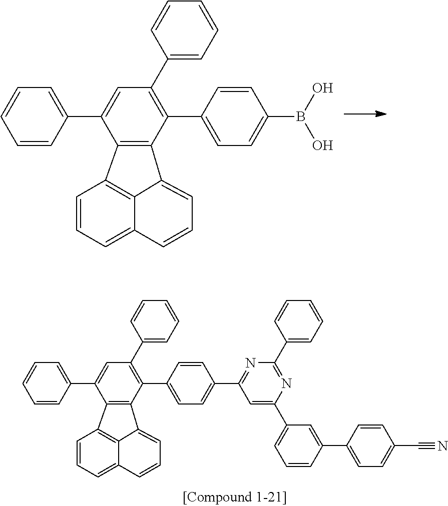 [SMILES: OB(O)c1ccc(-c2c(-c3ccccc3)cc(-c3ccccc3)c3c2-c2cccc4cccc-3c24)cc1 ; N#Cc1ccc(-c2cccc(-c3cc(-c4ccc(-c5c(-c6ccccc6)cc(-c6ccccc6)c6c5-c5cccc7cccc-6c57)cc4)nc(-c4ccccc4)n3)c2)cc1]

[Compound 1-21] was prepared in the same manner as in Synthesis of [Compound 1-5] except that (4-(8,10-diphenylfluoranthen-7-yl)phenyl)boronic acid was used instead of 8-(3-bromophenyl)-7,10-diphenylfluoranthene, and 3′-(6-chloro-2-phenylpyrimidin-4-yl)-[1,1′-biphenyl]-4-carbonitrile was used instead of (3-cyanophenyl)boronic acid.

[M+H]⁺=762

<Synthesis of Compound 1-22>

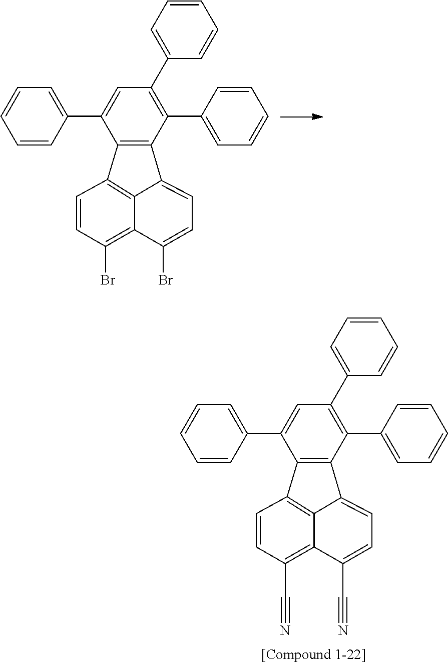 [SMILES: Brc1ccc2c3c(ccc(Br)c13)-c1c(-c3ccccc3)c(-c3ccccc3)cc(-c3ccccc3)c1-2 ; N#Cc1ccc2c3c(ccc(C#N)c13)-c1c(-c3ccccc3)c(-c3ccccc3)cc(-c3ccccc3)c1-2]

[Compound 1-22] was prepare in the same manner as in Synthesis of [Compound 1-1] except that 3,4-dibromo-7,8,10-triphenylfluoranthene was used instead of 8-(3-bromophenyl)-7,10-diphenylfluoranthene.

[M+H]⁺=481

<Synthesis of Compound 1-23>

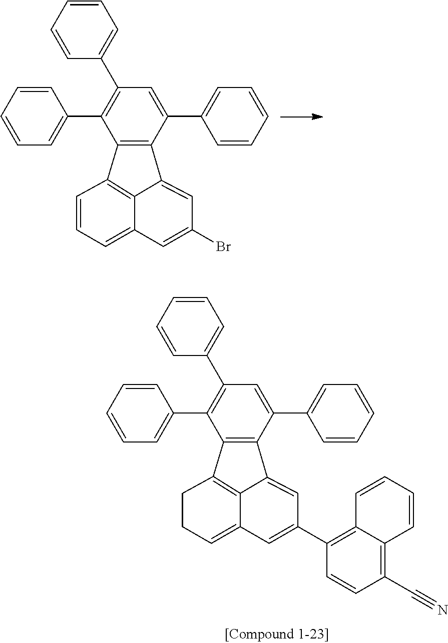 [SMILES: Brc1cc2c3c(cccc3c1)-c1c(-c3ccccc3)c(-c3ccccc3)cc(-c3ccccc3)c1-2 ; N#Cc1ccc(-c2cc3c4c(c2)=CCCC=4c2c(-c4ccccc4)c(-c4ccccc4)cc(-c4ccccc4)c2-3)c2ccccc12]

[Compound 1-23] was prepared in the same manner as in Synthesis of [Compound 1-5] except that 2-bromo-7,8,10-triphenylfluoranthene was used instead of 8-(3-bromophenyl)-7,10-diphenylfluoranthene, and (4-cyanonaphthalen-1-yl)boronic acid was used instead of (3-cyanophenyl)boronic acid.

[M+H]⁺=582

<Synthesis of Compound 1-24>

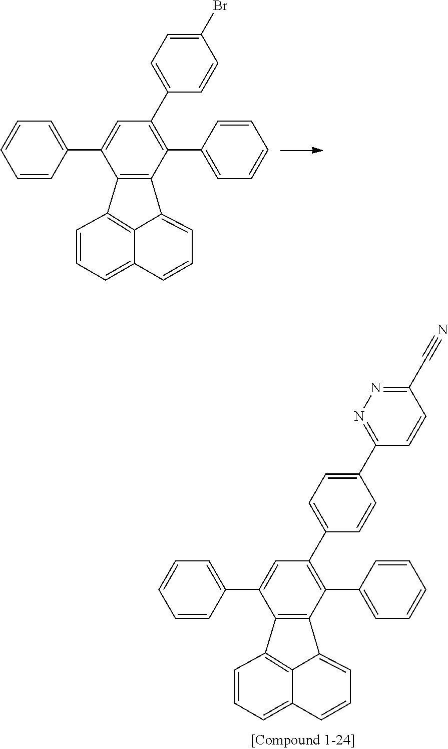 [SMILES: Brc1ccc(-c2cc(-c3ccccc3)c3c(c2-c2ccccc2)-c2cccc4cccc-3c24)cc1 ; N#Cc1ccc(-c2ccc(-c3cc(-c4ccccc4)c4c(c3-c3ccccc3)-c3cccc5cccc-4c35)cc2)nn1]

[Compound 1-24] was prepared in the same manner as in Synthesis of [Compound 1-5] except that 8-(4-bromophenyl)-7,10-diphenylfluoranthene was used instead of 8-(3-bromophenyl)-7,10-diphenylfluoranthene, and (6-cyanopyridazin-3-yl)boronic acid was used instead of (3-cyanophenyl)boronic acid.

[M+H]⁺=534

<Synthesis of Compound 1-25>

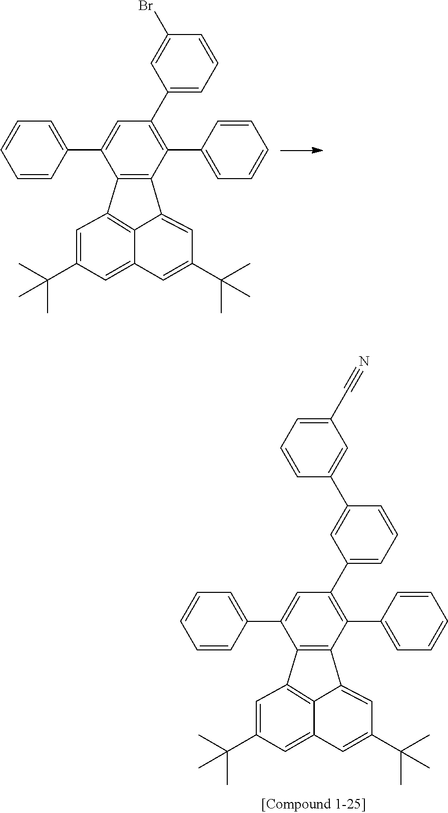 [SMILES: CC(C)(C)c1cc2c3c(cc(C(C)(C)C)cc3c1)-c1c(-c3ccccc3)c(-c3cccc(Br)c3)cc(-c3ccccc3)c1-2 ; CC(C)(C)c1cc2c3c(cc(C(C)(C)C)cc3c1)-c1c(-c3ccccc3)c(-c3cccc(-c4cccc(C#N)c4)c3)cc(-c3ccccc3)c1-2]

[Compound 1-25] was prepared in the same manner as in Synthesis of [Compound 1-5] except that 8-(3-bromophenyl)-2,5-di-tert-butyl-7,10-diphenylfluoranthene was used instead of 8-(3-bromophenyl)-7,10-diphenylfluoranthene.

[M+H]⁺=644

<Synthesis of Compound 1-26>

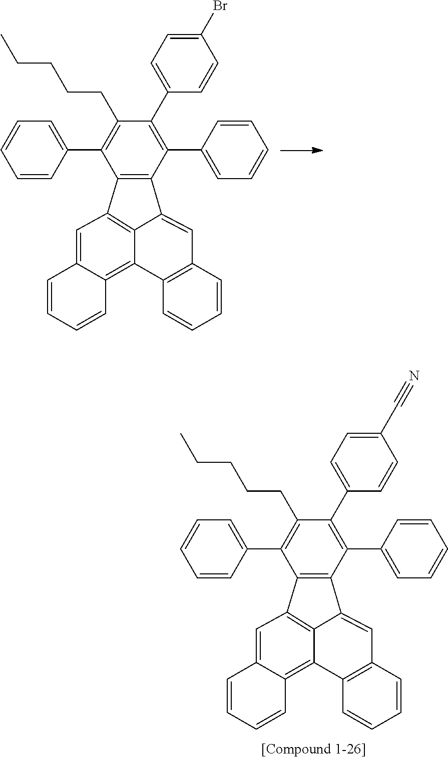 [SMILES: CCCCCc1c(-c2ccc(Br)cc2)c(-c2ccccc2)c2c(c1-c1ccccc1)-c1cc3ccccc3c3c1c-2cc1ccccc13 ; CCCCCc1c(-c2ccc(C#N)cc2)c(-c2ccccc2)c2c(c1-c1ccccc1)-c1cc3ccccc3c3c1c-2cc1ccccc13]

[Compound 1-26] was prepared in the same manner as in Synthesis of [Compound 1-1] except that 11-(4-bromophenyl)-12-pentyl-10,13-diphenyldibenzo[a,e]acephenanthrylene was used instead of 8-(3-bromophenyl)-7,10-diphenylfluoranthene.

[M+H]⁺=626

<Synthesis of Compound 1-27>

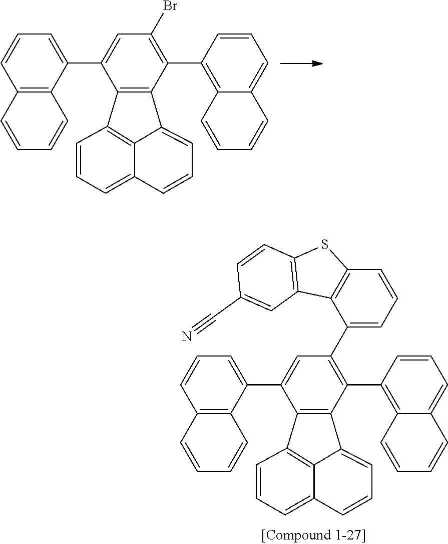 [SMILES: Brc1cc(-c2cccc3ccccc23)c2c(c1-c1cccc3ccccc13)-c1cccc3cccc-2c13 ; N#Cc1ccc2sc3cccc(-c4cc(-c5cccc6ccccc56)c5c(c4-c4cccc6ccccc46)-c4cccc6cccc-5c46)c3c2c1]

[Compound 1-27] was prepared in the same manner as in Synthesis of [Compound 1-5] except that 8-bromo-7,10-di(naphthalen-1-yl)fluoranthene was used instead of 8-(3-bromophenyl)-7,10-diphenylfluoranthene, and (8-cyanodibenzo[b,d]thiophen-1-yl)boronic acid was used instead of (3-cyanophenyl)boronic acid.

[M+H]⁺=662

<Synthesis of Compound 1-28>

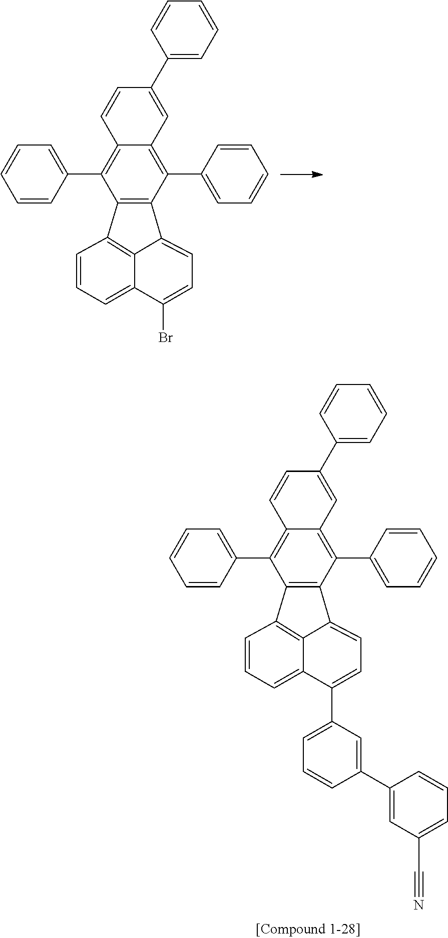 [SMILES: Brc1ccc2c3c(cccc13)-c1c-2c(-c2ccccc2)c2cc(-c3ccccc3)ccc2c1-c1ccccc1 ; N#Cc1cccc(-c2cccc(-c3ccc4c5c(cccc35)-c3c-4c(-c4ccccc4)c4cc(-c5ccccc5)ccc4c3-c3ccccc3)c2)c1]

[Compound 1-28] was prepared in the same manner as in Synthesis of [Compound 1-5] except that 3-bromo-7,10,12-triphenylbenzo[k]fluoranthene was used instead of 8-(3-bromophenyl)-7,10-diphenylfluoranthene, and (3′-cyano-[1,1′-biphenyl]-3-yl)boronic acid was used instead of (3-cyanophenyl)boronic acid.

[M+H]⁺=658

<Synthesis of Compound 1-29>

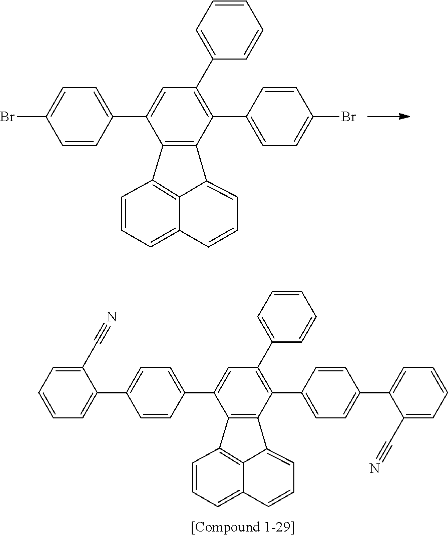 [SMILES: Brc1ccc(-c2cc(-c3ccccc3)c(-c3ccc(Br)cc3)c3c2-c2cccc4cccc-3c24)cc1 ; N#Cc1ccccc1-c1ccc(-c2cc(-c3ccccc3)c(-c3ccc(-c4ccccc4C#N)cc3)c3c2-c2cccc4cccc-3c24)cc1]

[Compound 1-29] was prepared in the same manner as in Synthesis of [Compound 1-5] except that 7,10-bis(4-bromophenyl)-8-phenylfluoranthene was used instead of 8-(3-bromophenyl)-7,10-diphenylfluoranthene, and (2-cyanophenyl)boronic acid was used instead of (3-cyanophenyl)boronic acid.

[M+H]⁺=633

<Synthesis of Compound 1-30>

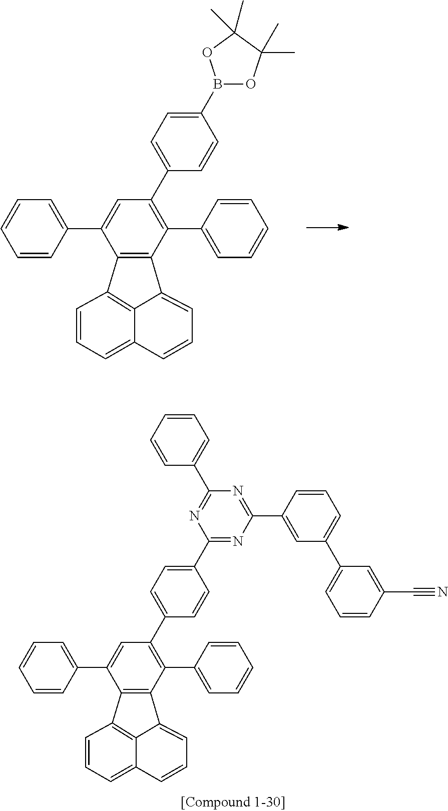 [SMILES: CC1(C)OB(c2ccc(-c3cc(-c4ccccc4)c4c(c3-c3ccccc3)-c3cccc5cccc-4c35)cc2)OC1(C)C ; N#Cc1cccc(-c2cccc(-c3nc(-c4ccccc4)nc(-c4ccc(-c5cc(-c6ccccc6)c6c(c5-c5ccccc5)-c5cccc7cccc-6c57)cc4)n3)c2)c1]

[Compound 1-30] was prepared in the same manner as in Synthesis of [Compound 1-5] except that 2-(4-(7,10-diphenylfluoranthen-8-yl)phenyl)-4,4,5,5-tetramethyl-1,3,2-dioxaborolane was used instead of 8-(3-bromophenyl)-7,10-diphenylfluoranthene, and 3′-(4-chloro-6-phenyl-1,3,5-triazin-2-yl)-[1,1′-biphenyl]-3-carbonitrile was used instead of (3-cyanophenyl)boronic acid.

[M+H]⁺=763

<Synthesis of Compound 1-31>

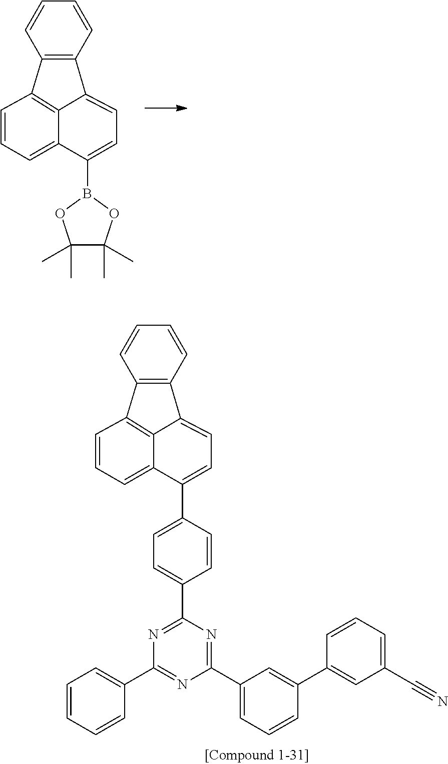 [SMILES: CC1(C)OB(c2ccc3c4c(cccc24)-c2ccccc2-3)OC1(C)C ; N#Cc1cccc(-c2cccc(-c3nc(-c4ccccc4)nc(-c4ccc(-c5ccc6c7c(cccc57)-c5ccccc5-6)cc4)n3)c2)c1]

[Compound 1-31] was prepared in the same manner as in Synthesis of [Compound 1-5] except that 2-(fluoranthen-3-yl)-4,4,5,5-tetramethyl-1,3,2-dioxaborolane was used instead of 8-(3-bromophenyl)-7,10-diphenylfluoranthene, and 3′-(4-(4-chlorophenyl)-6-phenyl-1,3,5-triazin-2-yl)-[1,1′-biphenyl]-3-carbonitrile was used instead of (3-cyanophenyl)boronic acid.

[M+H]⁺=611

<Synthesis of Compound 1-32>

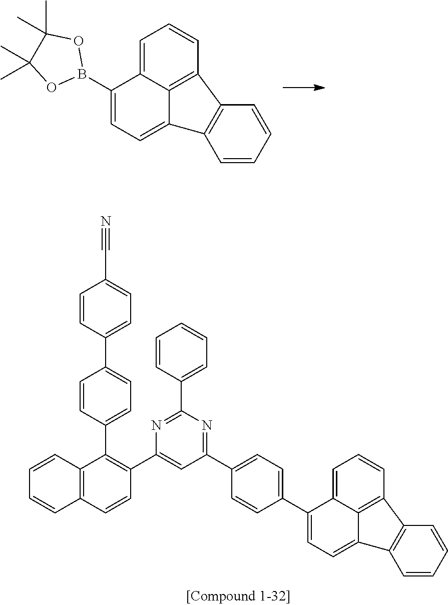 [SMILES: CC1(C)OB(c2ccc3c4c(cccc24)-c2ccccc2-3)OC1(C)C ; N#Cc1ccc(-c2ccc(-c3c(-c4cc(-c5ccc(-c6ccc7c8c(cccc68)-c6ccccc6-7)cc5)nc(-c5ccccc5)n4)ccc4ccccc34)cc2)cc1]

[Compound 1-32] was prepared in the same manner as in Synthesis of [Compound 1-5] except that 2-(fluoranthen-3-yl)-4,4,5,5-tetramethyl-1,3,2-dioxaborolane was used instead of 8-(3-bromophenyl)-7,10-diphenylfluoranthene, and 4′-(2-(6-(4-chlorophenyl)-2-phenylpyrimidin-4-yl) naphthalen-1-yl)-[1,1′-biphenyl]-4-carbonitrile was used instead of (3-cyanophenyl) boronic acid.

[M+H]⁺=736

<Synthesis of Compound 1-33>

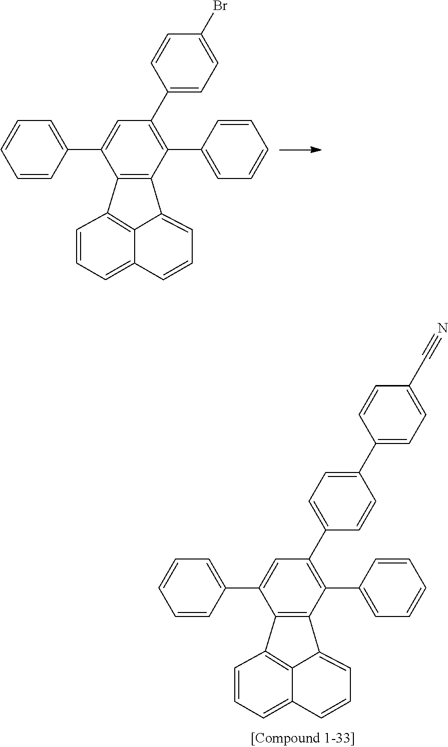 [SMILES: Brc1ccc(-c2cc(-c3ccccc3)c3c(c2-c2ccccc2)-c2cccc4cccc-3c24)cc1 ; N#Cc1ccc(-c2ccc(-c3cc(-c4ccccc4)c4c(c3-c3ccccc3)-c3cccc5cccc-4c35)cc2)cc1]

[Compound 1-33] was prepared in the same manner as in Synthesis of [Compound 1-3] except that 8-(4-bromophenyl)-7,10-diphenylfluoranthene was used instead of [Compound 1-3-A], and (4-cyanophenyl)boronic acid was used instead of 2-(tert-butyl)-4,4,5,5-tetramethyl-1,3,2-dioxaborolane.

[M+H]⁺=532

<Synthesis of Compound 1-34>

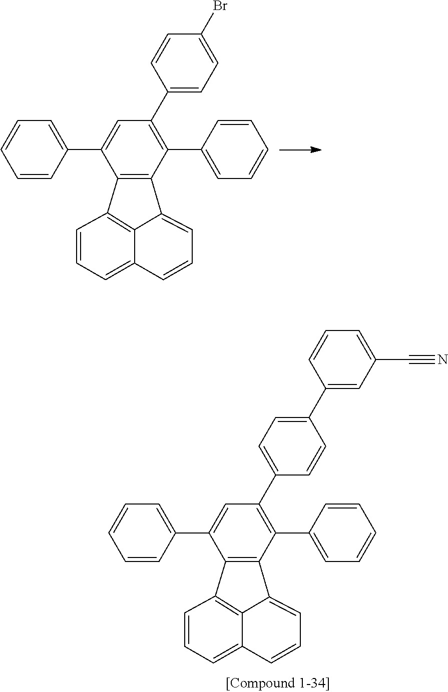 [SMILES: Brc1ccc(-c2cc(-c3ccccc3)c3c(c2-c2ccccc2)-c2cccc4cccc-3c24)cc1 ; N#Cc1cccc(-c2ccc(-c3cc(-c4ccccc4)c4c(c3-c3ccccc3)-c3cccc5cccc-4c35)cc2)c1]

[Compound 1-34] was prepared in the same manner as in Synthesis of [Compound 1-3] except that 8-(4-bromophenyl)-7,10-diphenylfluoranthene was used instead of [Compound 1-3-A], and (3-cyanophenyl)boronic acid was used instead of 2-(tert-butyl)-4,4,5,5-tetramethyl-1,3,2-dioxaborolane.

[M+H]⁺=532

<Synthesis of Compound 1-35>

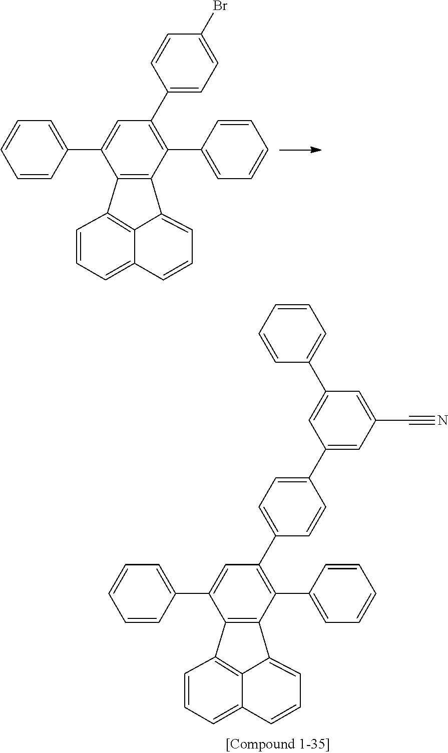 [SMILES: Brc1ccc(-c2cc(-c3ccccc3)c3c(c2-c2ccccc2)-c2cccc4cccc-3c24)cc1 ; N#Cc1cc(-c2ccccc2)cc(-c2ccc(-c3cc(-c4ccccc4)c4c(c3-c3ccccc3)-c3cccc5cccc-4c35)cc2)c1]

[Compound 1-35] was prepared in the same manner as in Synthesis of [Compound 1-3] except that 8-(4-bromophenyl)-7,10-diphenylfluoranthene was used instead of [Compound 1-3-A], and (5-cyano-[1,1′-biphenyl]-3-yl)boronic acid was used instead of 2-(tert-butyl)-4,4,5,5-tetramethyl-1,3,2-dioxaborolane.

[M+H]⁺=608

<Synthesis of Compound 1-36>

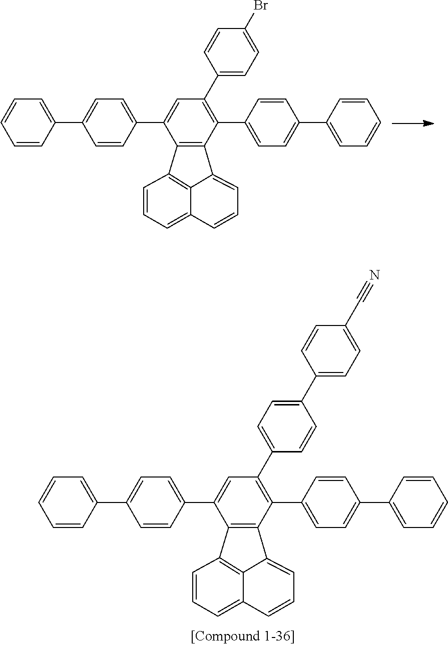 [SMILES: Brc1ccc(-c2cc(-c3ccc(-c4ccccc4)cc3)c3c(c2-c2ccc(-c4ccccc4)cc2)-c2cccc4cccc-3c24)cc1 ; N#Cc1ccc(-c2ccc(-c3cc(-c4ccc(-c5ccccc5)cc4)c4c(c3-c3ccc(-c5ccccc5)cc3)-c3cccc5cccc-4c35)cc2)cc1]

[Compound 1-36] was prepared in the same manner as in Synthesis of [Compound 1-3] except that 7,10-di([1,1′-biphenyl]-4-yl)-8-(4-bromophenyl)fluoranthene was used instead of [Compound 1-3-A], and (4-cyanophenyl)boronic acid was used instead of 2-(tert-butyl)-4,4,5,5-tetramethyl-1,3,2-dioxaborolane.

[M+H]⁺=684

Manufacture of Organic Light Emitting Device

### Example 1

A glass substrate on which indium tin oxide (ITO) was coated as a thin film to a thickness of 100 nm was placed in distilled water containing dissolved detergent and ultrasonically cleaned. Herein, a product of Fischer Co. was used as the detergent, and as the distilled water, distilled water filtered twice with a filter manufactured by Millipore Co. was used. After the ITO was cleaned for 30 minutes, ultrasonic cleaning was repeated twice using distilled water for 10 minutes. After the cleaning with distilled water was finished, the substrate was ultrasonically cleaned with solvents of isopropyl alcohol, acetone and methanol, then dried, and then transferred to a plasma cleaner. In addition, the substrate was cleaned for 5 minutes using oxygen plasma, and then transferred to a vacuum deposition apparatus.

On the transparent ITO electrode prepared as above, the following Compound HI-A was thermal vacuum deposited to a thickness of 60 nm to form a hole injection layer.

On the hole injection layer, the following Compound HAT was vacuum deposited to form a first hole transfer layer having a thickness of 5 nm, and the following Compound HT-A was vacuum deposited on the first hole transfer layer to form a second hole transfer layer having a thickness of 50 nm.

Subsequently, the following Compound BH and Compound BD were vacuum deposited on the second hole transfer layer in a weight ratio of 25:1 to form a light emitting layer having a thickness of 20 nm.

On the light emitting layer, [Compound 1-1] and the following Compound LiQ were vacuum deposited in a weight ratio of 1:1 to form an electron injection and transfer layer having a thickness of 35 nm.

A cathode was formed by depositing lithium fluoride (LiF) on the electron injection and transfer layer to a thickness of 1 nm and then depositing aluminum to a thickness of 100 nm, and as a result, an organic light emitting device was manufactured.

In the above-described process, the deposition rates of the organic materials were maintained at 0.04 nm/sec to 0.09 nm/sec, the deposition rate of the lithium fluoride was maintained at 0.03 nm/sec, and the deposition rate of the aluminum was maintained at 0.2 nm/sec. The degree of vacuum during the deposition was maintained at 1×10⁻⁷ torr to 5×10⁻⁵ torr.

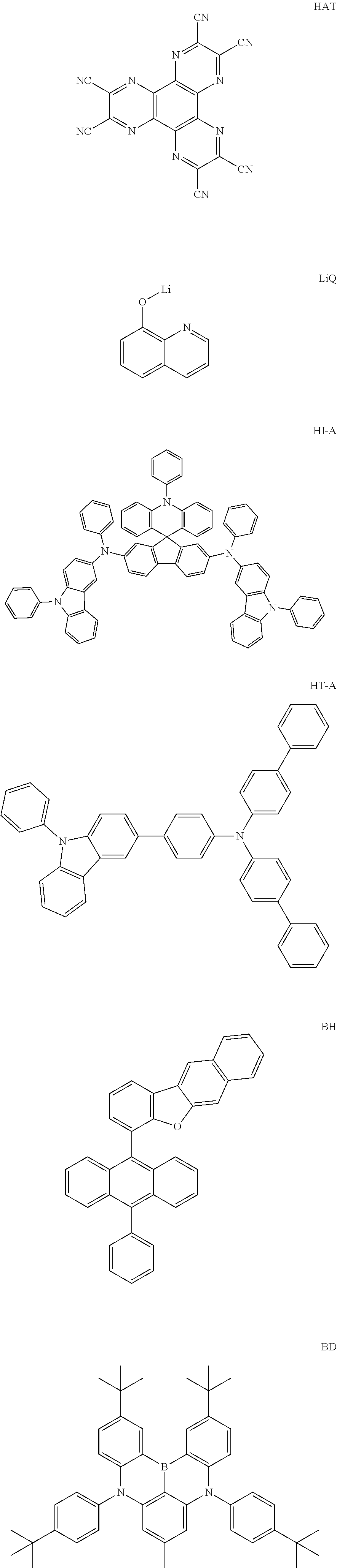

### Examples 2 to 36

Organic light emitting devices were manufactured in the same manner as in Example 1 except that compounds of the following Table 1 were used instead of [Compound 1-1] of Example 1.

### Comparative Examples 1 to 9

Organic light emitting devices were manufactured in the same manner as in Example 1 except that compounds of the following Table 1 were used instead of [Compound 1-1] of Example 1.

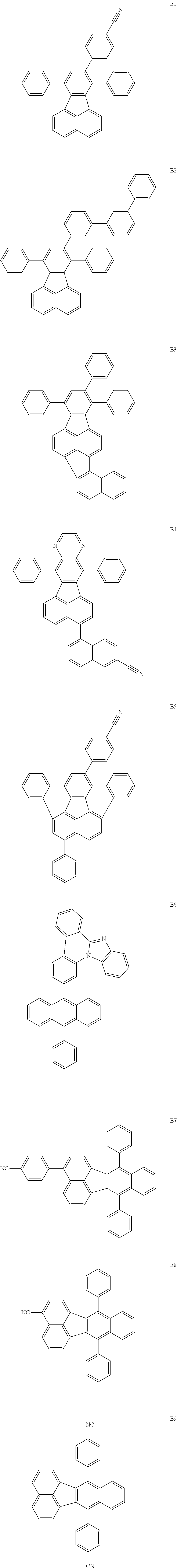

For each of the organic light emitting devices of Examples 1 to 36 and Comparative Examples 1 to 9, voltage and efficiency were measured under current density of 10 mA/cm² and lifetime (LT₉₅) was measured under current density of 20 mA/cm², and the results are shown in the following Table 1. Herein, LT₉₅ means time taken for luminance to become 95% with respect to initial luminance. Color coordinate (x, y) means a CIE color coordinate.

TABLE 1

| | | Driv- | | | |
|---|---|---|---|---|---|
| | Electron | ing | | | |
| | Injection | Volt- | Effi- | Color | |
| | and Transfer | age | ciency | Coordinate | LT₉₅ |
| | Layer | (V) | (cd/A) | (x, y) | (h) |
| Example 1 | Compound 1-1 | 3.98 | 4.95 | (0.142, 0.097) | 296 |
| Example 2 | Compound 1-2 | 4.10 | 4.84 | (0.142, 0.097) | 278 |
| Example 3 | Compound 1-3 | 4.03 | 4.97 | (0.142, 0.098) | 302 |
| Example 4 | Compound 1-4 | 4.04 | 4.90 | (0.142, 0.096) | 259 |
| Example 5 | Compound 1-5 | 4.01 | 5.00 | (0.142, 0.097) | 290 |
| Example 6 | Compound 1-6 | 4.07 | 5.00 | (0.142, 0.097) | 295 |
| Example 7 | Compound 1-7 | 3.99 | 4.98 | (0.142, 0.096) | 288 |
| Example 8 | Compound 1-8 | 4.05 | 5.02 | (0.142, 0.096) | 279 |
| Example 9 | Compound 1-9 | 4.02 | 5.01 | (0.142, 0.097) | 283 |
| Example 10 | Compound 1-10 | 3.92 | 4.96 | (0.142, 0.097) | 257 |
| Example 11 | Compound 1-11 | 4.13 | 4.92 | (0.142, 0.097) | 271 |
| Example 12 | Compound 1-12 | 4.03 | 5.03 | (0.142, 0.096) | 287 |
| Example 13 | Compound 1-13 | 4.06 | 5.02 | (0.142, 0.096) | 290 |
| Example 14 | Compound 1-14 | 3.97 | 5.08 | (0.142, 0.096) | 294 |
| Example 15 | Compound 1-15 | 4.02 | 5.01 | (0.142, 0.097) | 284 |
| Example 16 | Compound 1-16 | 4.00 | 4.94 | (0.142, 0.096) | 288 |
| Example 17 | Compound 1-17 | 4.08 | 4.85 | (0.142, 0.097) | 300 |
| Example 18 | Compound 1-18 | 4.03 | 4.96 | (0.142, 0.096) | 286 |
| Example 19 | Compound 1-19 | 4.07 | 5.00 | (0.142, 0.097) | 292 |
| Example 20 | Compound 1-20 | 4.12 | 4.99 | (0.142, 0.097) | 287 |
| Example 21 | Compound 1-21 | 3.99 | 5.10 | (0.142, 0.097) | 293 |
| Example 22 | Compound 1-22 | 4.06 | 4.89 | (0.142, 0.097) | 305 |
| Example 23 | Compound 1-23 | 4.04 | 4.97 | (0.142, 0.097) | 291 |
| Example 24 | Compound 1-24 | 4.05 | 4.92 | (0.142, 0.097) | 279 |
| Example 25 | Compound 1-25 | 3.99 | 5.03 | (0.142, 0.097) | 284 |
| Example 26 | Compound 1-26 | 4.06 | 4.88 | (0.142, 0.097) | 262 |
| Example 27 | Compound 1-27 | 4.09 | 4.99 | (0.142, 0.097) | 270 |
| Example 28 | Compound 1-28 | 4.04 | 5.00 | (0.142, 0.097) | 289 |
| Example 29 | Compound 1-29 | 4.02 | 4.94 | (0.142, 0.097) | 297 |
| Example 30 | Compound 1-30 | 4.00 | 5.05 | (0.142, 0.097) | 306 |
| Example 31 | Compound 1-31 | 3.89 | 5.07 | (0.142, 0.097) | 279 |
| Example 32 | Compound 1-32 | 3.93 | 5.08 | (0.142, 0.097) | 298 |
| Example 33 | Compound 1-33 | 4.02 | 5.04 | (0.142, 0.097) | 290 |
| Example 34 | Compound 1-34 | 3.99 | 5.08 | (0.142, 0.097) | 286 |
| Example 35 | Compound 1-35 | 4.04 | 5.03 | (0.142, 0.097) | 294 |
| Example 36 | Compound 1-36 | 4.08 | 5.00 | (0.142, 0.097) | 301 |
| Comparative | Compound E1 | 4.16 | 4.51 | (0.142, 0.097) | 200 |
| Example 1 | | | | | |
| Comparative | Compound E2 | 4.23 | 4.48 | (0.142, 0.097) | 113 |
| Example 2 | | | | | |
| Comparative | Compound E3 | 4.19 | 4.44 | (0.142, 0.096) | 98 |
| Example 3 | | | | | |
| Comparative | Compound E4 | 4.27 | 4.29 | (0.142, 0.097) | 136 |
| Example 4 | | | | | |
| Comparative | Compound E5 | 4.30 | 4.12 | (0.142, 0.097) | 129 |
| Example 5 | | | | | |
| Comparative | Compound E6 | 4.25 | 4.39 | (0.142, 0.097) | 106 |
| Example 6 | | | | | |
| Comparative | Compound E7 | 4.21 | 4.47 | (0.142, 0.097) | 202 |
| Example 7 | | | | | |
| Comparative | Compound E8 | 4.18 | 4.50 | (0.142, 0.097) | 185 |
| Example 8 | | | | | |
| Comparative | Compound E9 | 4.22 | 4.42 | (0.142, 0.097) | 178 |
| Example 9 | | | | | |

In Table 1, it was identified that Examples 1 to 36 using Chemical Formula 1 according to one embodiment of the present specification in the electron injection and transfer layer of the organic light emitting device had low driving voltage, high efficiency and excellent lifetime properties compared to Comparative Examples 1 to 9. Particularly, it was identified that Example 1 using the compound in which R1 or R2 is Chemical Formula A, l1 is 1, m is 1, L1 is a phenylene group, and the cyano group bonds at a meta position of the phenylene group in Chemical Formula 1 of the present specification in the organic light emitting device had low driving voltage, high efficiency and excellent lifetime properties compared to Comparative Example 1 using the compound in which the cyano group bonds at a para position of the phenylene group in the organic light emitting device. This is due to the fact that, when the cyano group bonds at a meta or ortho position, the distance of electron migration between the cyano group and the phenyl group becomes relatively shorter allowing active electron migration compared to in the compound having the cyano group bonding at a para position.

In addition, it was identified that, since R1 and R2 smoothly control electron mobility by including substituents other than hydrogen in Examples 17, 18 and 28, low driving voltage, high efficiency and excellent lifetime properties were obtained compared to Comparative Examples 7 to 9 using the compound in which m is 1 and R1 and R2 are hydrogen in Chemical Formula 1 of the present specification.

Hereinbefore, preferred embodiments of the present disclosure have been described, however, the present disclosure is not limited thereto, and various modifications can be made within the scope of the claims and the scope of the detailed descriptions, and these also fall within the category of the present disclosure.

1. A compound of Chemical Formula 1: 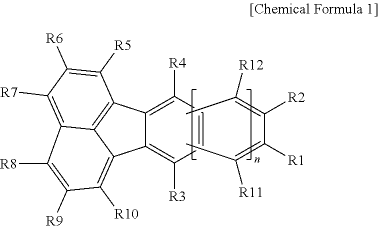
wherein, in Chemical Formula 1;
n is 0 or 1;
when n is 0, at least one of R1 to R10 is a group of the following Chemical Formula A;
when n is 1, at least one of R1 to R12 is a group of the following Chemical Formula A; and
the rest of the groups among R1 to R12 other than the group of the following Chemical Formula A are the same as or different from each other, and each independently is hydrogen, deuterium, a halogen group, a cyano group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group or a substituted or unsubstituted heteroaryl group, or adjacent groups bond to each other to form a substituted or unsubstituted ring; 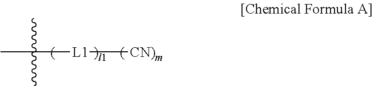
in Chemical Formula A,
L1 is a direct bond, a substituted or unsubstituted arylene group, or a substituted or unsubstituted heteroarylene group;
l1 is an integer of 1 to 5;
m is an integer of 1 to 3;
when l1 is 2 or greater, the two or more Lis are the same as or different from each other; 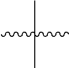
means a site directly bonding to at least one of R1 to R10 or at least one of R1 to R12 of Chemical Formula 1;
when n is 1 and R1 and R2 are the rest of the groups other than the group of Chemical Formula A, at least one of R1 and R2 is deuterium, a halogen group, a cyano group, a nitro group, a hydroxyl group, a substituted or unsubstituted alkyl group a substituted or unsubstituted cycloalkyl group, a substituted or unsubstituted alkoxy group a substituted or unsubstituted aryloxy group a substituted or unsubstituted alkylthioxy group, a substituted or unsubstituted arylthioxy group, a substituted or unsubstituted alkylsulfoxy group, a substituted or unsubstituted arylsulfoxy group a substituted or unsubstituted alkenyl group a substituted or unsubstituted silyl group, a substituted or unsubstituted boron group, a substituted or unsubstituted amine group, a substituted or unsubstituted arylphosphine group, a substituted or unsubstituted phosphine oxide group a substituted or unsubstituted aryl group or a substituted or unsubstituted heteroaryl group, or adjacent groups bond to each other to form a substituted or unsubstituted; and
when n is 0, R1 or R2 is the group of Chemical Formula A, l1 is 1, m is 1 and L1 is a phenylene group, R1 or R2 is a group of the following Chemical Formula A-1 or A-2; 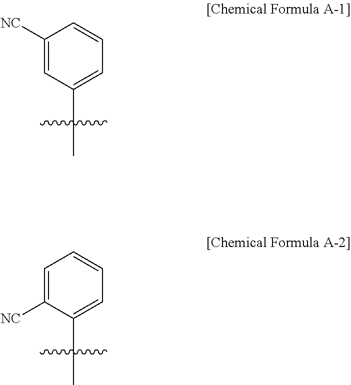 [SMILES: *c1cccc(C#N)c1 ; *c1ccccc1C#N]
wherein in Chemical Formulae A-1 and A-2; 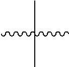
means a site directly bonding to R1 or R2 of Chemical Formula 1.

2. The compound of claim 1, wherein Chemical Formula 1 is the following Chemical Formula 1-1 or 1-2: 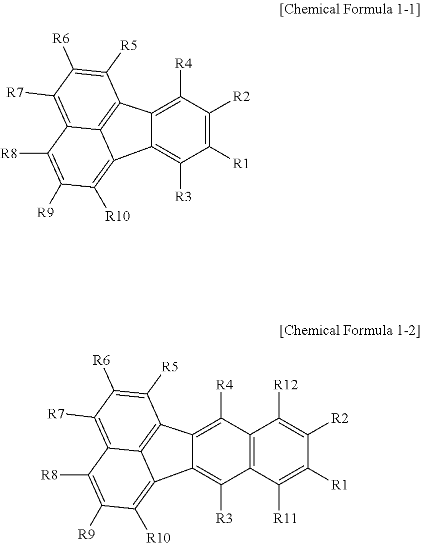
wherein in Chemical Formulae 1-1 and 1-2;
R1 to R12 have the same definitions as in Chemical Formula 1.

3. The compound of claim 1, wherein Chemical Formula A is any one of the following Chemical Formulae A-3 to A-5: 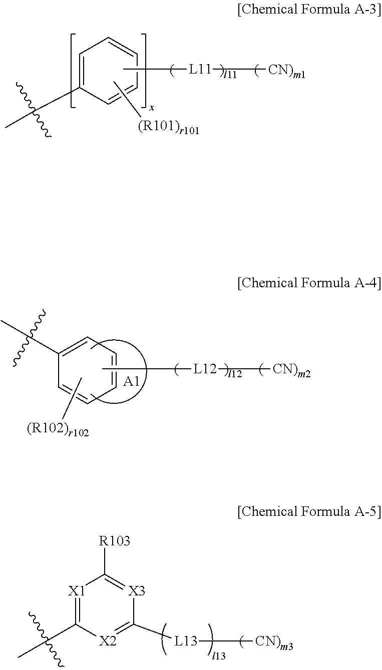
wherein in Chemical Formulae A-3 to A-5;
x is 0 or 1;
L11 to L13 are the same as or different from each other, and each independently is a direct bond, a substituted or unsubstituted arylene group, or a substituted or unsubstituted heteroarylene group;
r101 is an integer of 1 to 4;
r102 is an integer of 1 to 3;
R101 and R102 are the same as or different from each other, and each independently is hydrogen, deuterium, a halogen group, a cyano group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group, or bond to adjacent groups to form a substituted or unsubstituted ring;
when r101 is 2 or greater, the two or more R101s are the same as or different from each other;
when r102 is 2 or greater, the two or more R102s are the same as or different from each other;
A1 is a substituted or unsubstituted hydrocarbon ring or a substituted or unsubstituted heteroring;
at least one of X1 to X3 is N, and the rest are CR;
R and R103 are the same as or different from each other, and each independently is hydrogen, deuterium, a halogen group, a cyano group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group, or adjacent two or more groups among R, R103 and L13 bond to each other to form a substituted or unsubstituted ring;
m1 to m3 are each an integer of 1 to 3;
l11 is an integer of 1 to 4;
l12 is an integer of 1 to 4;
l13 is an integer of 1 to 4;
when l11 is 2 or greater, the two or more L11s are the same as or different from each other;
when l12 is 2 or greater, the two or more L12s are the same as or different from each other; and
when l13 is 2 or greater, the two or more L13s are the same as or different from each other.

4. The compound of claim 1, wherein the rest of the groups among R1 to R12 other than the group of Chemical Formula A are the same as or different from each other, and each independently is hydrogen; a linear or branched alkyl group having 1 to 30 carbon atoms; a monocyclic or polycyclic aryl group having 6 to 30 carbon atoms that is unsubstituted or substituted with a linear or branched alkyl group having 1 to 30 carbon atoms, a linear or branched alkoxy group having 1 to 30 carbon atoms, a linear or branched haloalkyl group having 1 to 30 carbon atoms, a monocyclic or polycyclic aryl group having 6 to 30 carbon atoms, or a monocyclic or polycyclic heteroaryl group having 2 to 30 carbon atoms that is unsubstituted or substituted with a monocyclic or polycyclic aryl group having 6 to 30 carbon atoms; or a monocyclic or polycyclic heteroaryl group having 2 to 30 carbon atoms that is unsubstituted or substituted with a monocyclic or polycyclic aryl group having 6 to 30 carbon atoms, or adjacent groups bond to each other to form a monocyclic or polycyclic aromatic hydrocarbon ring having 6 to 30 carbon atoms; and
L1 is a direct bond; an arylene group having 6 to 30 carbon atoms that is unsubstituted or substituted with a linear or branched alkyl group having 1 to 30 carbon atoms or a monocyclic or polycyclic aryl group having 6 to 30 carbon atoms; or a monocyclic or polycyclic heteroarylene group having 2 to 30 carbon atoms that is unsubstituted or substituted with a monocyclic or polycyclic aryl group having 6 to 30 carbon atoms unsubstituted or substituted with a cyano group.

5. The compound of claim 1, wherein the compound of Chemical Formula 1 is any one compound selected from among the following compounds:

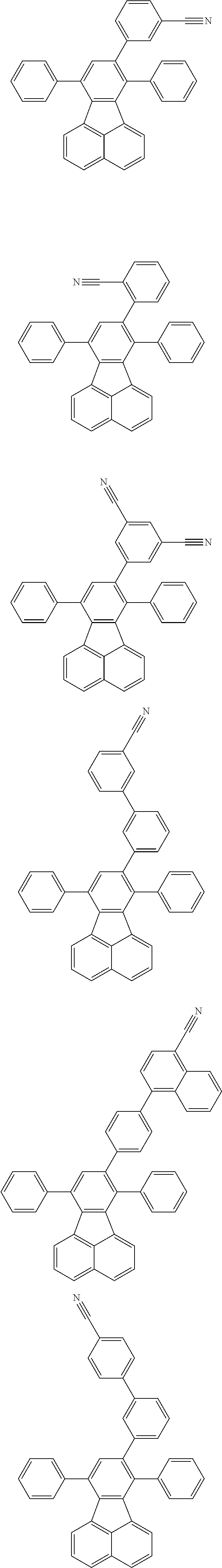

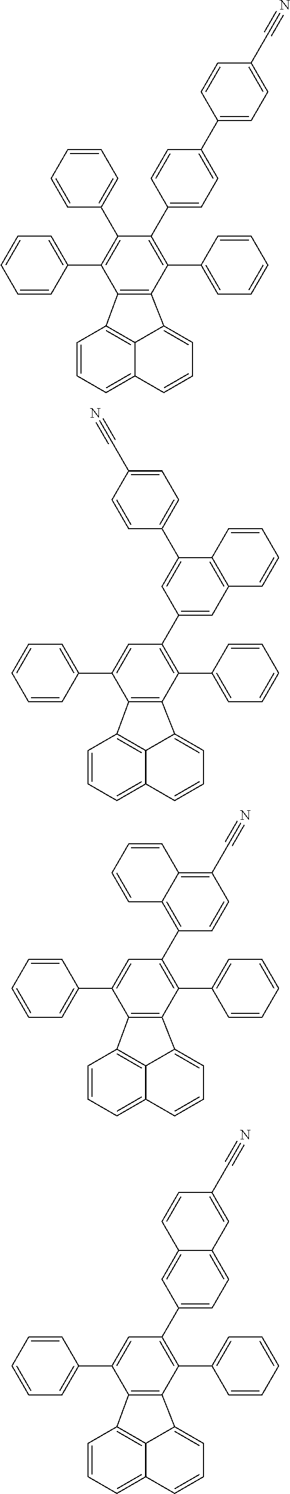

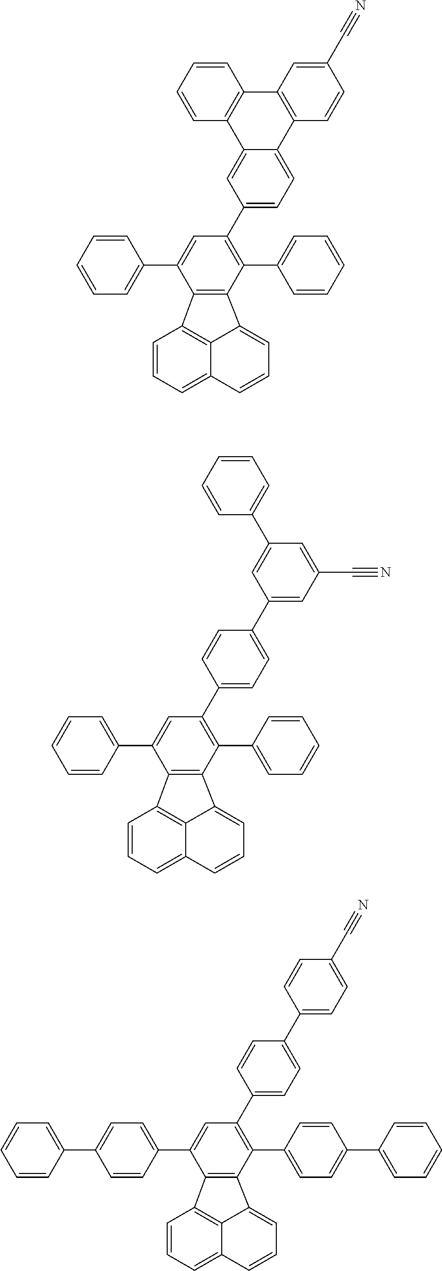 [SMILES: N#Cc1ccc2c3ccc(-c4cc(-c5ccccc5)c5c(c4-c4ccccc4)-c4cccc6cccc-5c46)cc3c3ccccc3c2c1 ; N#Cc1cc(-c2ccccc2)cc(-c2ccc(-c3cc(-c4ccccc4)c4c(c3-c3ccccc3)-c3cccc5cccc-4c35)cc2)c1 ; N#Cc1ccc(-c2ccc(-c3cc(-c4ccc(-c5ccccc5)cc4)c4c(c3-c3ccc(-c5ccccc5)cc3)-c3cccc5cccc-4c35)cc2)cc1]

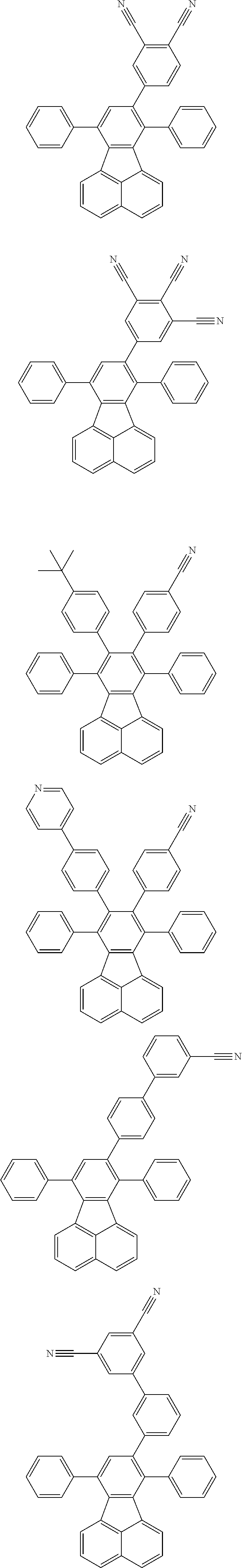

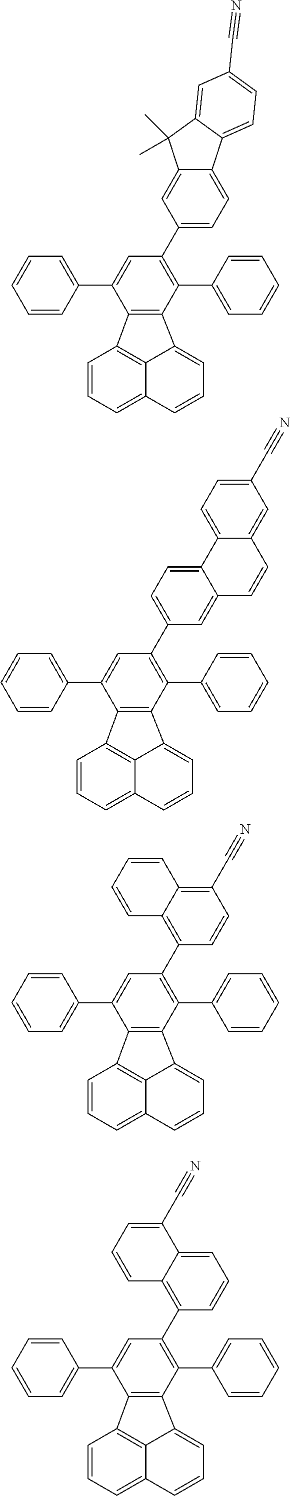

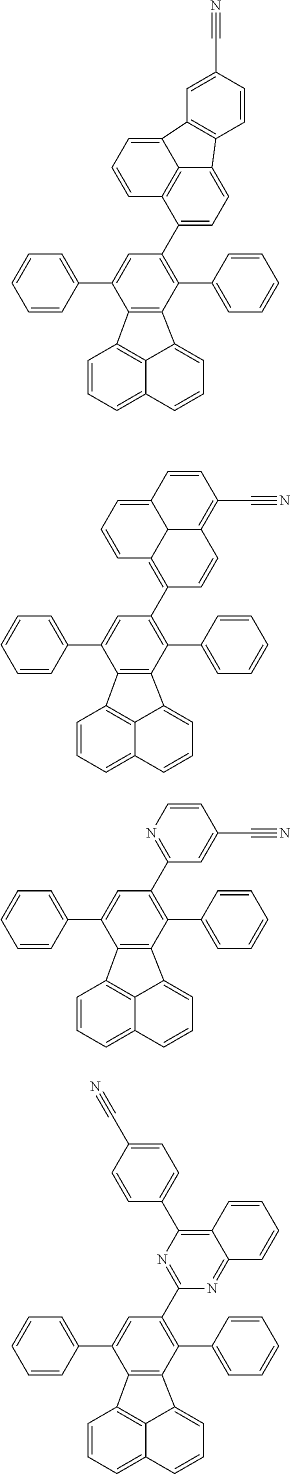

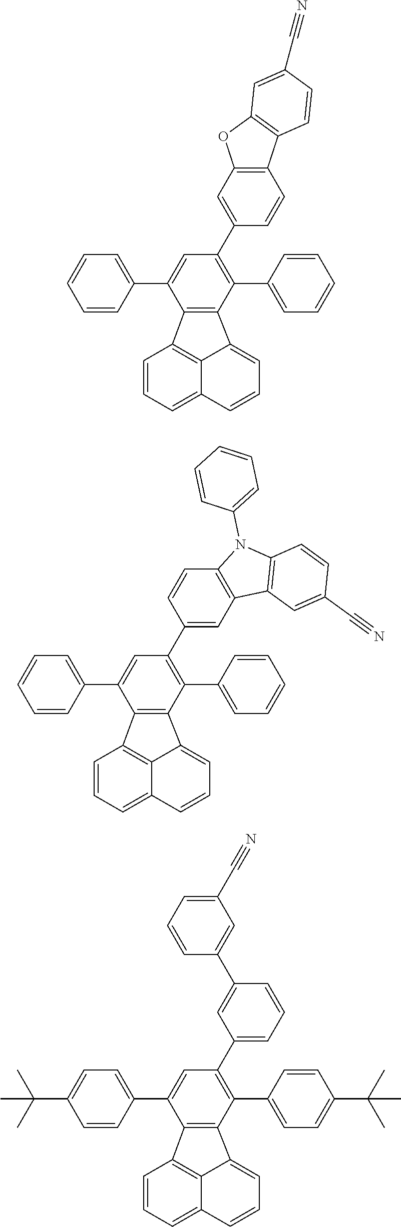

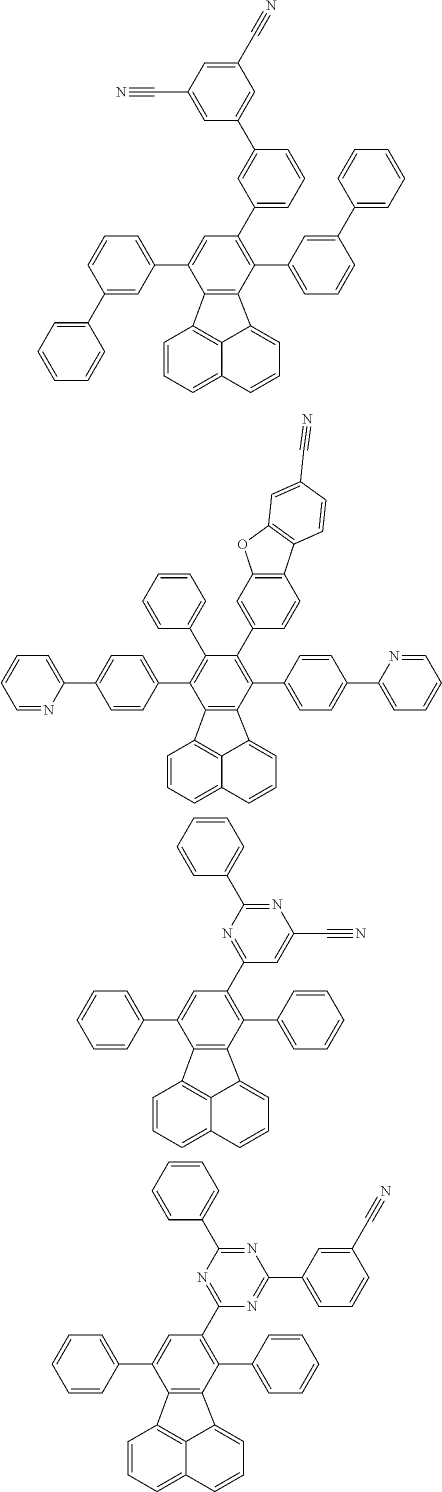

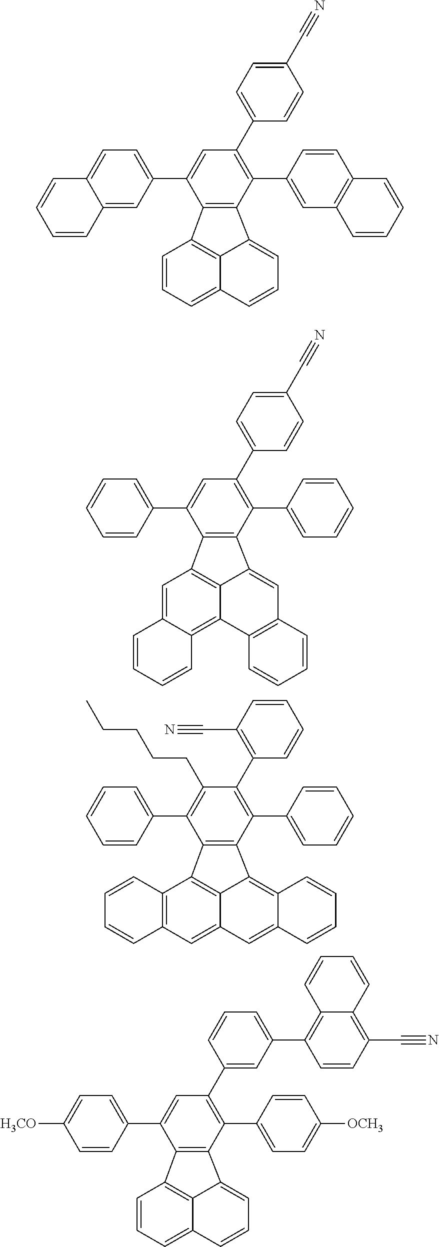

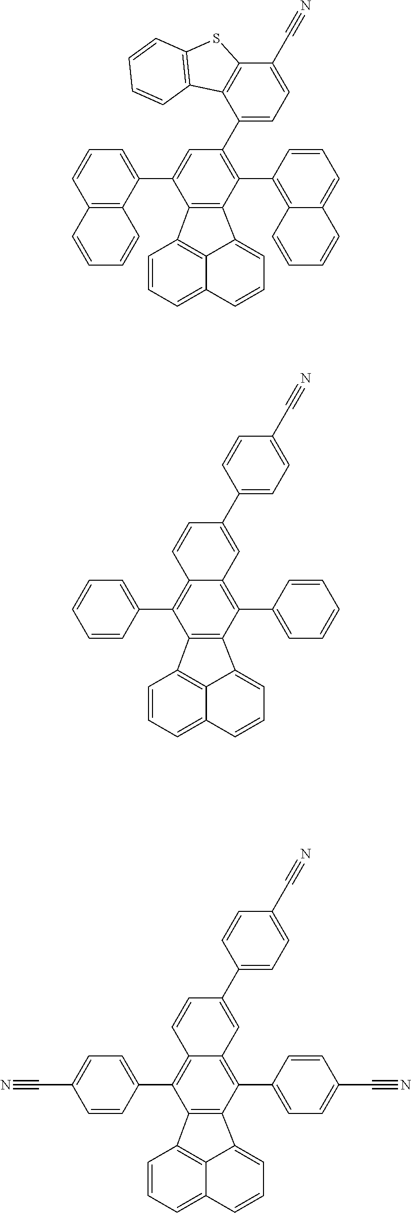 [SMILES: N#Cc1ccc(-c2cc(-c3cccc4ccccc34)c3c(c2-c2cccc4ccccc24)-c2cccc4cccc-3c24)c2c1sc1ccccc12 ; N#Cc1ccc(-c2ccc3c(-c4ccccc4)c4c(c(-c5ccccc5)c3c2)-c2cccc3cccc-4c23)cc1 ; N#Cc1ccc(-c2ccc3c(-c4ccc(C#N)cc4)c4c(c(-c5ccc(C#N)cc5)c3c2)-c2cccc3cccc-4c23)cc1]

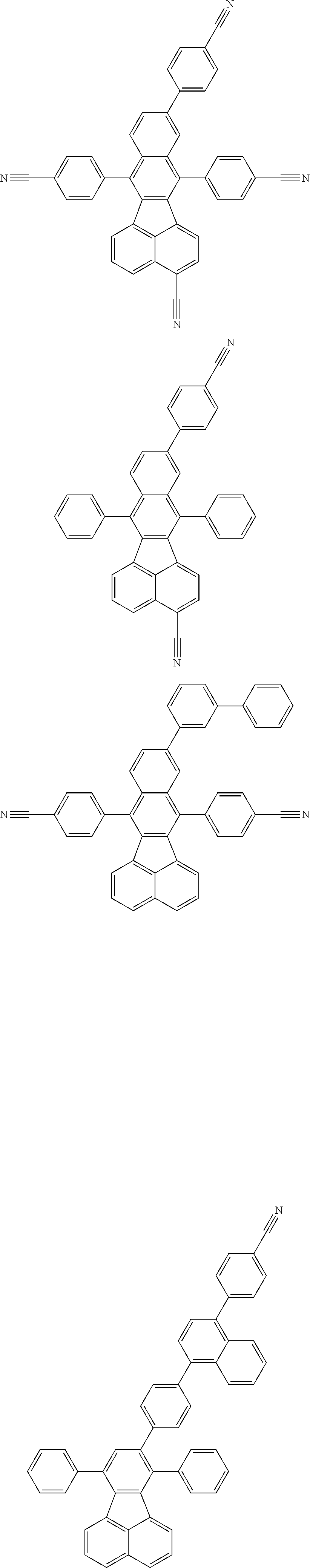

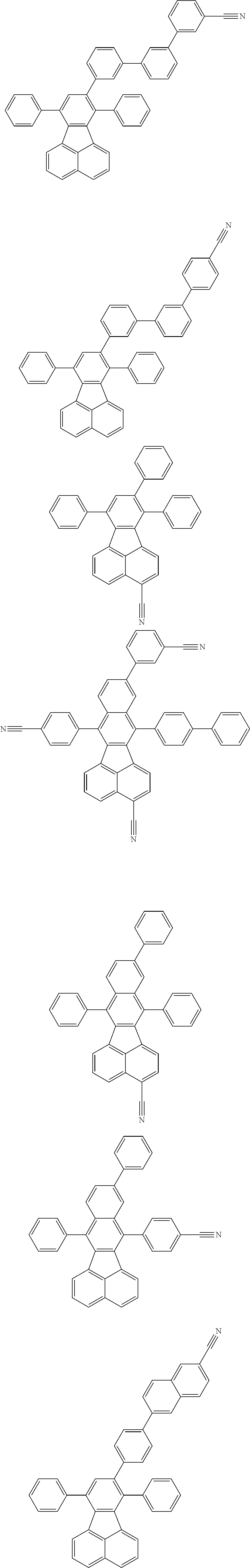

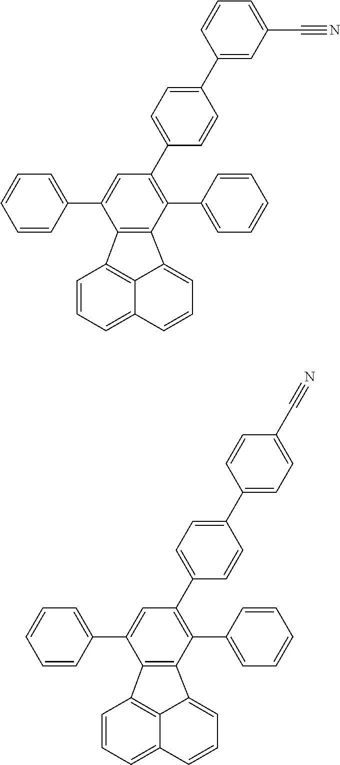 [SMILES: N#Cc1cccc(-c2ccc(-c3cc(-c4ccccc4)c4c(c3-c3ccccc3)-c3cccc5cccc-4c35)cc2)c1 ; N#Cc1ccc(-c2ccc(-c3cc(-c4ccccc4)c4c(c3-c3ccccc3)-c3cccc5cccc-4c35)cc2)cc1]

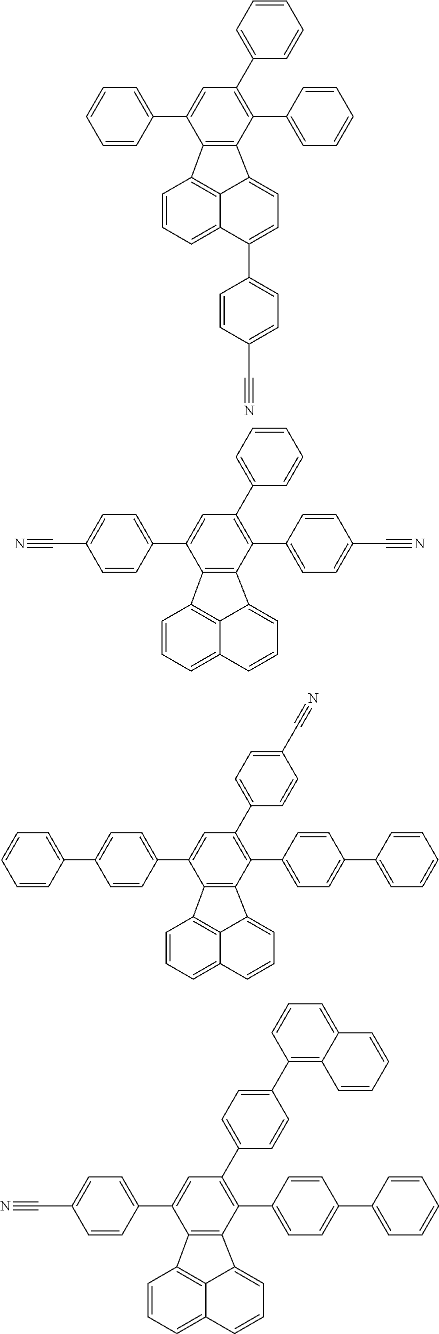

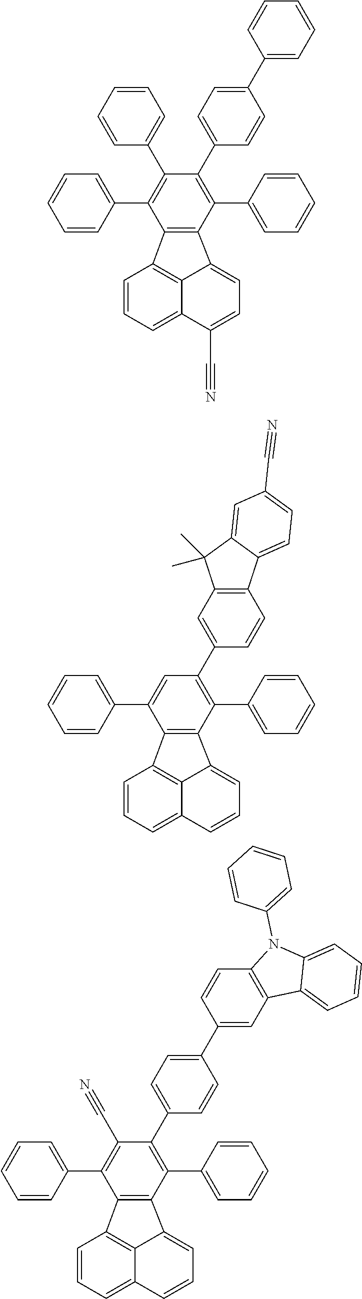

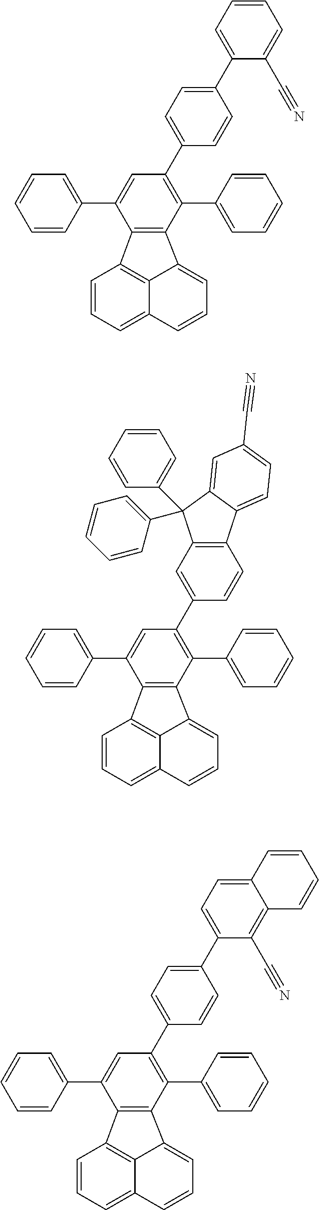 [SMILES: N#Cc1ccccc1-c1ccc(-c2cc(-c3ccccc3)c3c(c2-c2ccccc2)-c2cccc4cccc-3c24)cc1 ; N#Cc1ccc2c(c1)C(c1ccccc1)(c1ccccc1)c1cc(-c3cc(-c4ccccc4)c4c(c3-c3ccccc3)-c3cccc5cccc-4c35)ccc1-2 ; N#Cc1c(-c2ccc(-c3cc(-c4ccccc4)c4c(c3-c3ccccc3)-c3cccc5cccc-4c35)cc2)ccc2ccccc12]

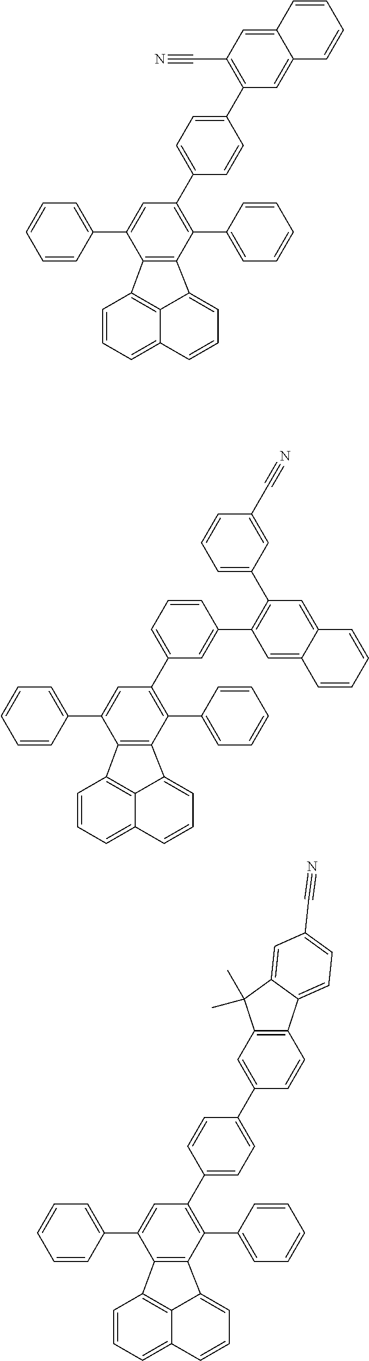 [SMILES: N#Cc1cc2ccccc2cc1-c1ccc(-c2cc(-c3ccccc3)c3c(c2-c2ccccc2)-c2cccc4cccc-3c24)cc1 ; N#Cc1cccc(-c2cc3ccccc3cc2-c2cccc(-c3cc(-c4ccccc4)c4c(c3-c3ccccc3)-c3cccc5cccc-4c35)c2)c1 ; CC1(C)c2cc(C#N)ccc2-c2ccc(-c3ccc(-c4cc(-c5ccccc5)c5c(c4-c4ccccc4)-c4cccc6cccc-5c46)cc3)cc21]

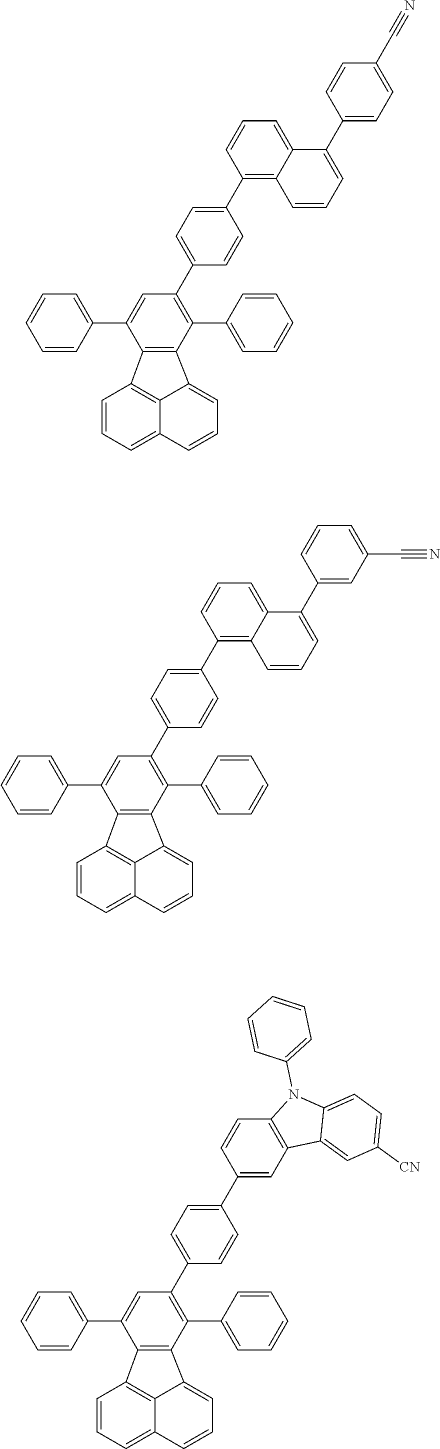 [SMILES: N#Cc1ccc(-c2cccc3c(-c4ccc(-c5cc(-c6ccccc6)c6c(c5-c5ccccc5)-c5cccc7cccc-6c57)cc4)cccc23)cc1 ; N#Cc1cccc(-c2cccc3c(-c4ccc(-c5cc(-c6ccccc6)c6c(c5-c5ccccc5)-c5cccc7cccc-6c57)cc4)cccc23)c1 ; N#Cc1ccc2c(c1)c1cc(-c3ccc(-c4cc(-c5ccccc5)c5c(c4-c4ccccc4)-c4cccc6cccc-5c46)cc3)ccc1n2-c1ccccc1]

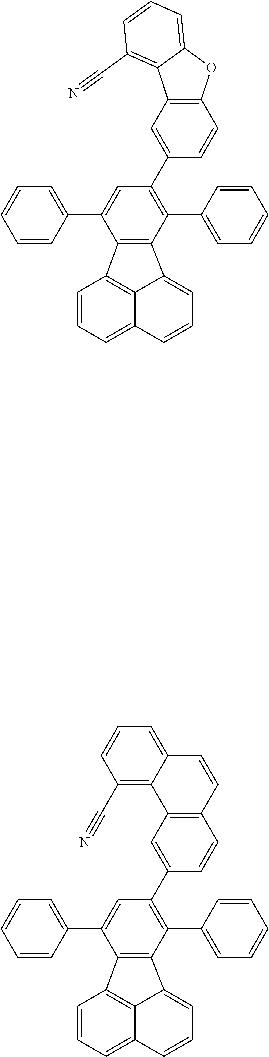 [SMILES: N#Cc1cccc2oc3ccc(-c4cc(-c5ccccc5)c5c(c4-c4ccccc4)-c4cccc6cccc-5c46)cc3c12 ; N#Cc1cccc2ccc3ccc(-c4cc(-c5ccccc5)c5c(c4-c4ccccc4)-c4cccc6cccc-5c46)cc3c12]

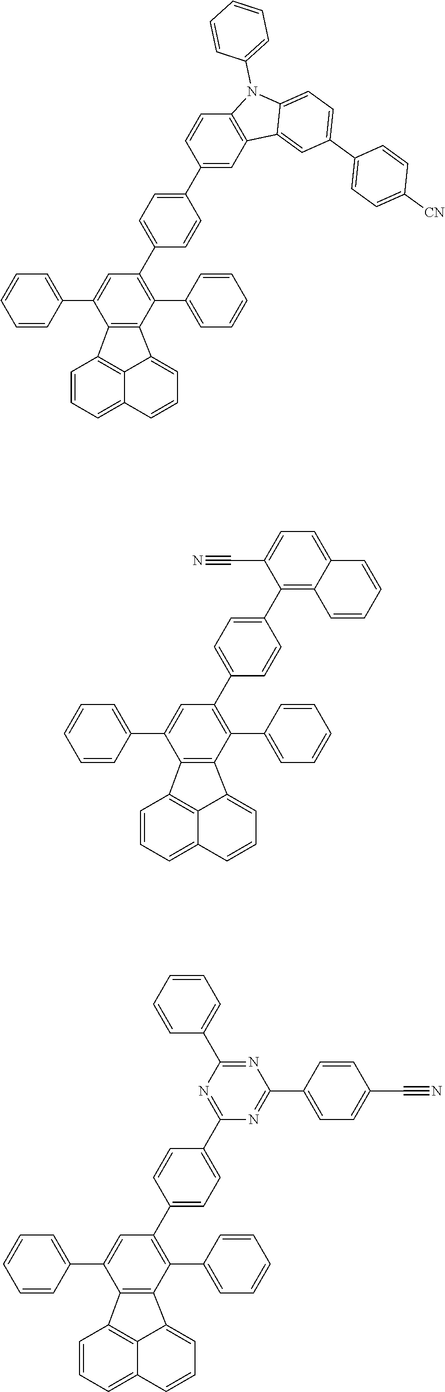 [SMILES: N#Cc1ccc(-c2ccc3c(c2)c2cc(-c4ccc(-c5cc(-c6ccccc6)c6c(c5-c5ccccc5)-c5cccc7cccc-6c57)cc4)ccc2n3-c2ccccc2)cc1 ; N#Cc1ccc2ccccc2c1-c1ccc(-c2cc(-c3ccccc3)c3c(c2-c2ccccc2)-c2cccc4cccc-3c24)cc1 ; N#Cc1ccc(-c2nc(-c3ccccc3)nc(-c3ccc(-c4cc(-c5ccccc5)c5c(c4-c4ccccc4)-c4cccc6cccc-5c46)cc3)n2)cc1]

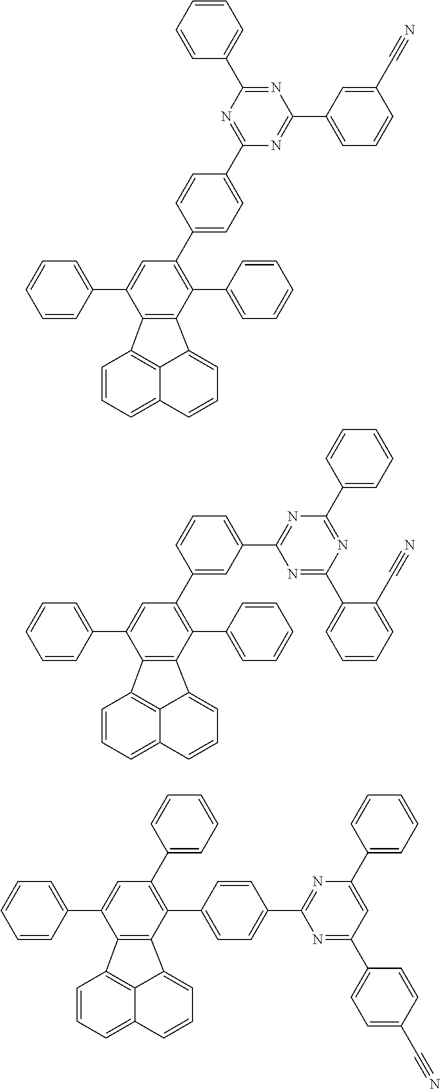

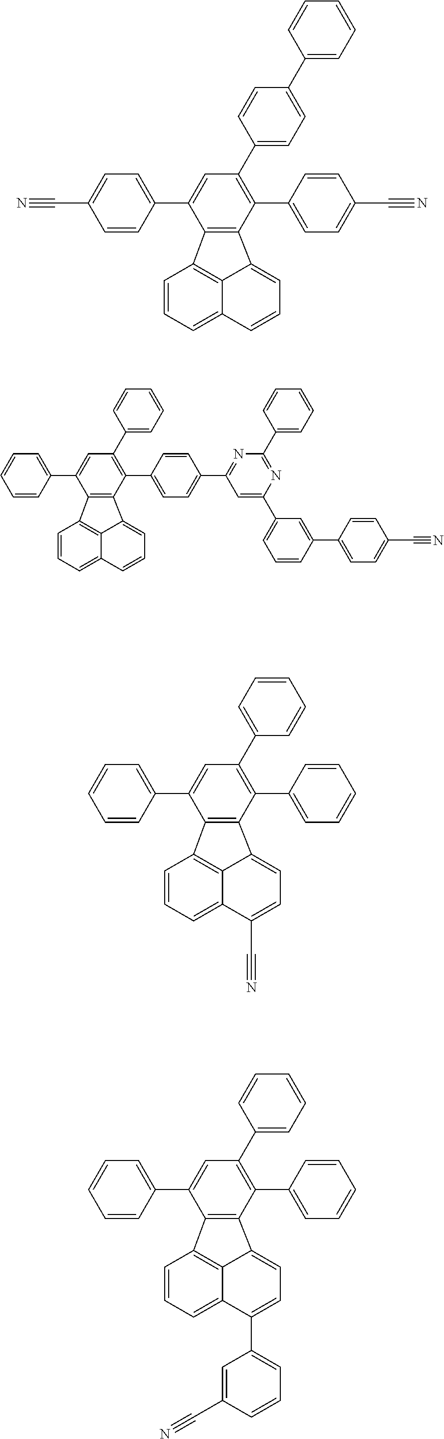 [SMILES: N#Cc1ccc(-c2cc(-c3ccc(-c4ccccc4)cc3)c(-c3ccc(C#N)cc3)c3c2-c2cccc4cccc-3c24)cc1 ; N#Cc1ccc(-c2cccc(-c3cc(-c4ccc(-c5c(-c6ccccc6)cc(-c6ccccc6)c6c5-c5cccc7cccc-6c57)cc4)nc(-c4ccccc4)n3)c2)cc1 ; N#Cc1ccc2c3c(cccc13)-c1c(-c3ccccc3)cc(-c3ccccc3)c(-c3ccccc3)c1-2 ; N#Cc1cccc(-c2ccc3c4c(cccc24)-c2c(-c4ccccc4)cc(-c4ccccc4)c(-c4ccccc4)c2-3)c1]

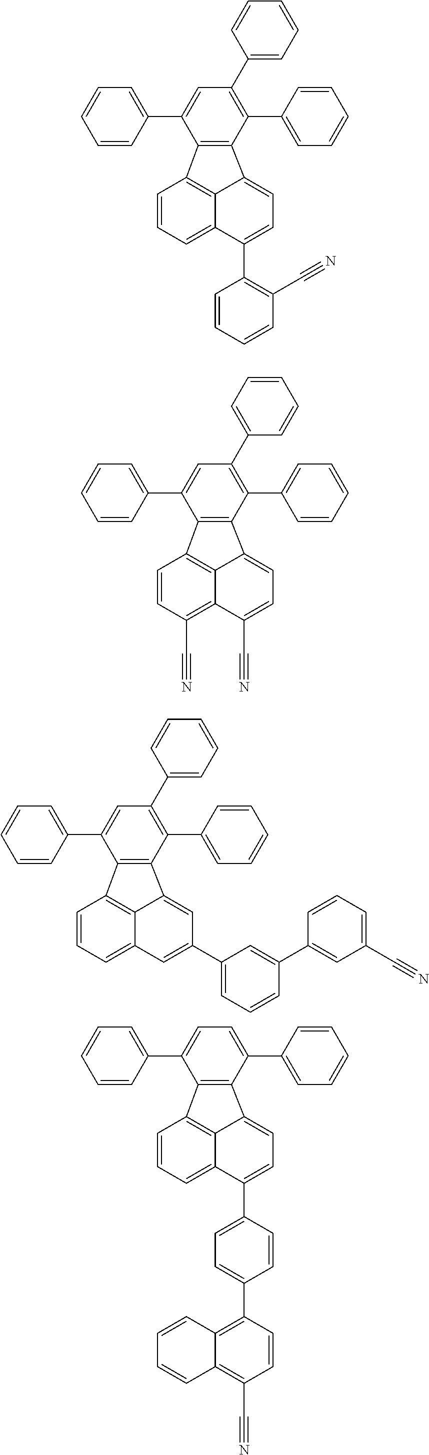

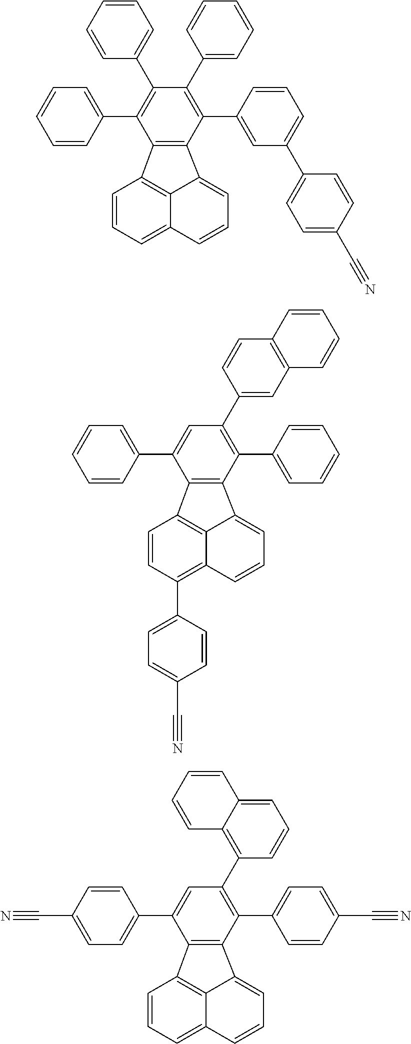

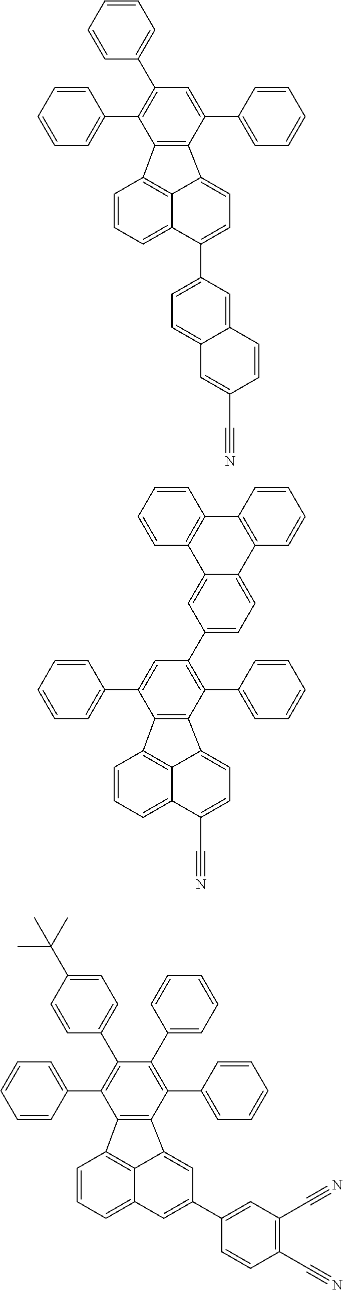

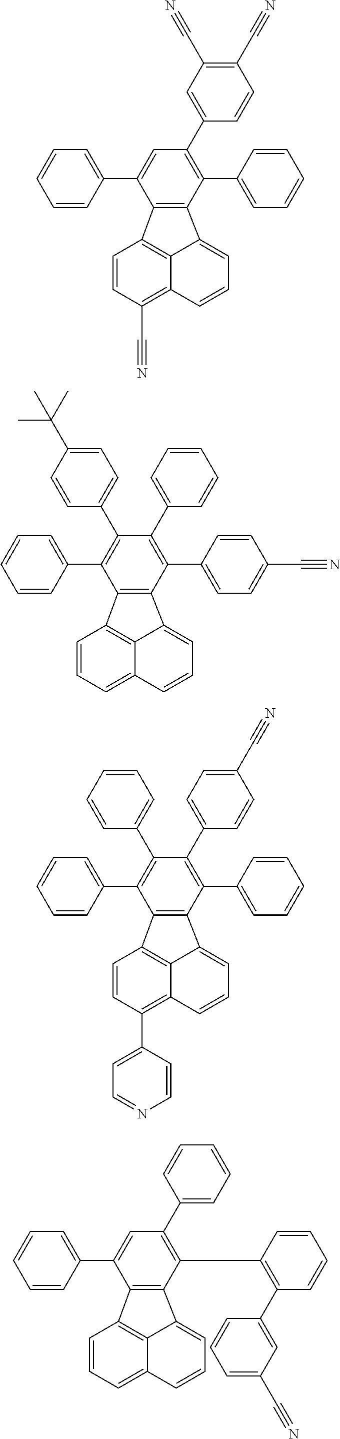 [SMILES: N#Cc1ccc(-c2cc(-c3ccccc3)c3c(c2-c2ccccc2)-c2cccc4c(C#N)ccc-3c24)cc1C#N ; CC(C)(C)c1ccc(-c2c(-c3ccccc3)c(-c3ccc(C#N)cc3)c3c(c2-c2ccccc2)-c2cccc4cccc-3c24)cc1 ; N#Cc1ccc(-c2c(-c3ccccc3)c(-c3ccccc3)c3c(c2-c2ccccc2)-c2cccc4c(-c5ccncc5)ccc-3c24)cc1 ; N#Cc1cccc(-c2ccccc2-c2c(-c3ccccc3)cc(-c3ccccc3)c3c2-c2cccc4cccc-3c24)c1]

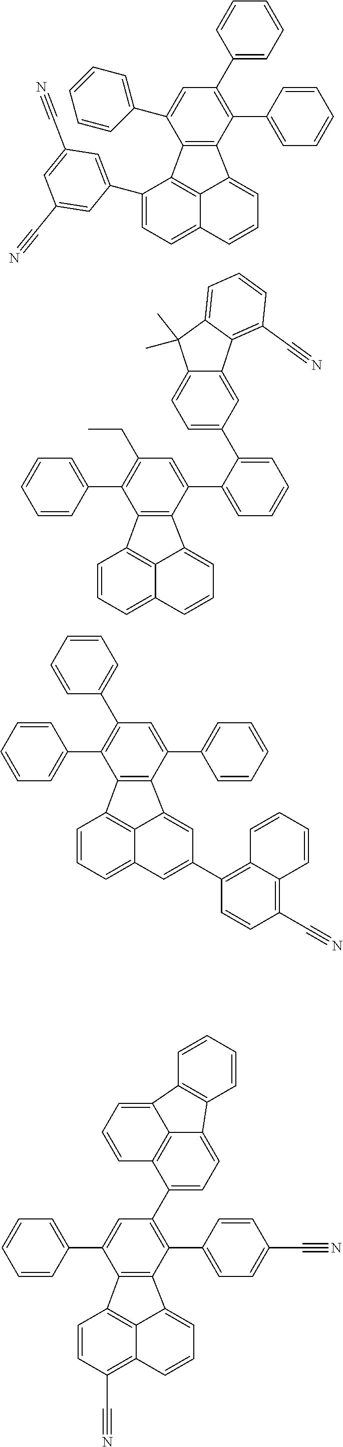

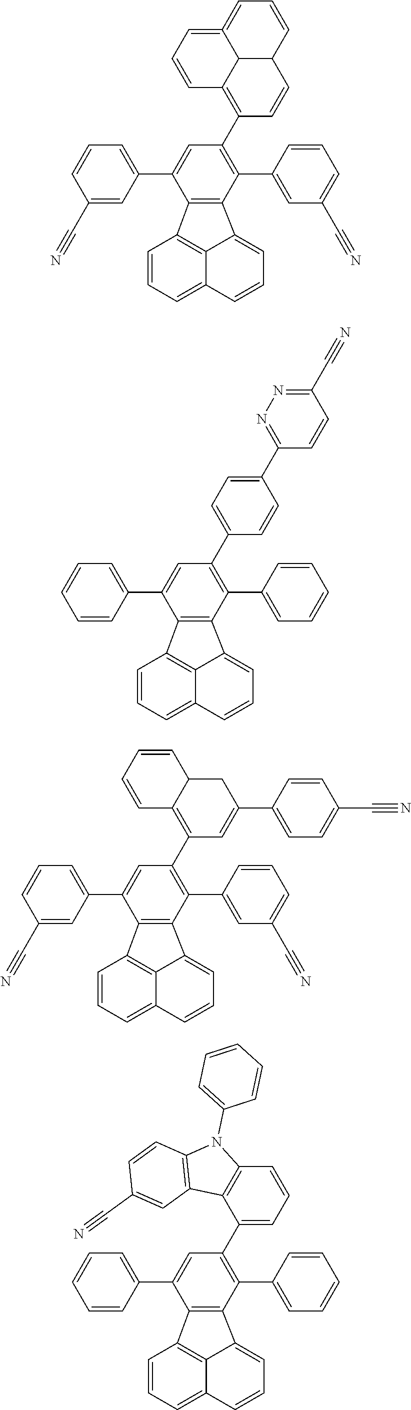

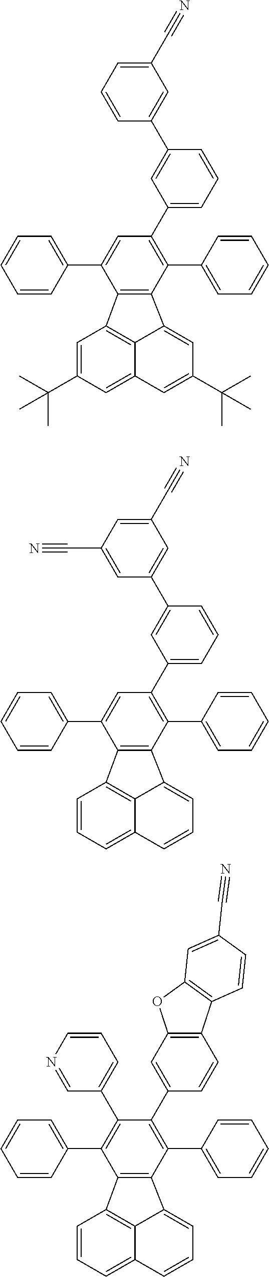 [SMILES: CC(C)(C)c1cc2c3c(cc(C(C)(C)C)cc3c1)-c1c(-c3ccccc3)c(-c3cccc(-c4cccc(C#N)c4)c3)cc(-c3ccccc3)c1-2 ; N#Cc1cc(C#N)cc(-c2cccc(-c3cc(-c4ccccc4)c4c(c3-c3ccccc3)-c3cccc5cccc-4c35)c2)c1 ; N#Cc1ccc2c(c1)oc1cc(-c3c(-c4cccnc4)c(-c4ccccc4)c4c(c3-c3ccccc3)-c3cccc5cccc-4c35)ccc12]

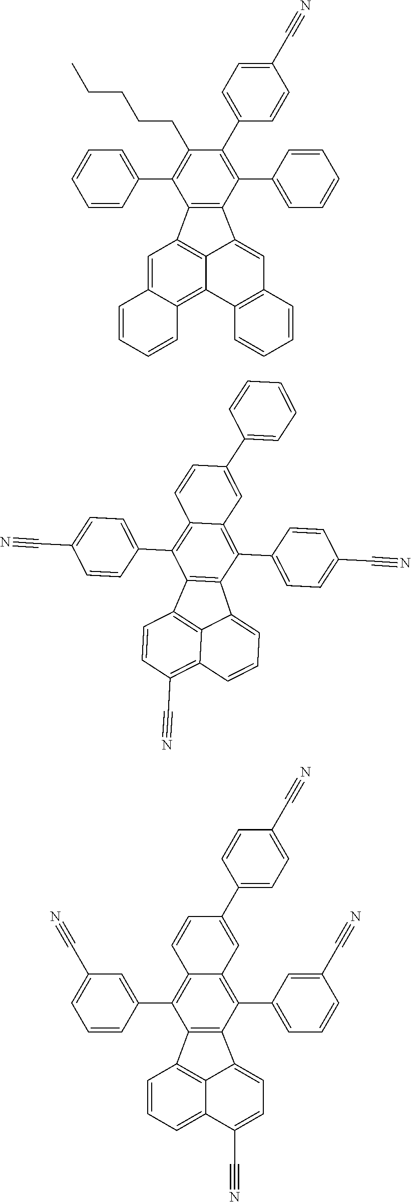

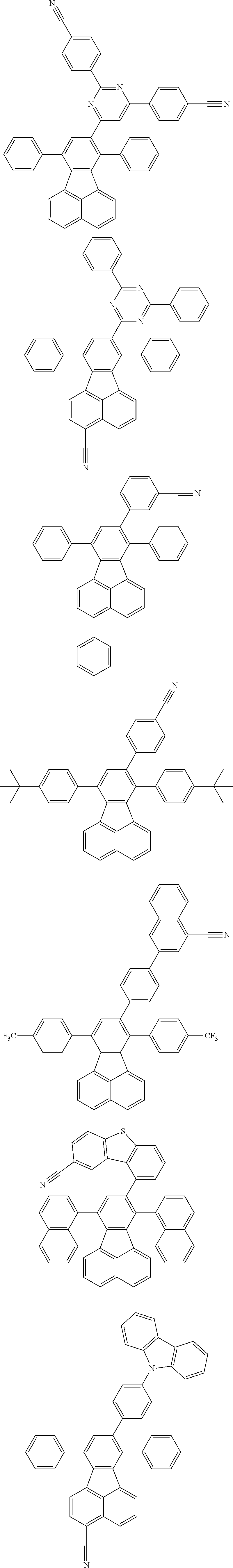

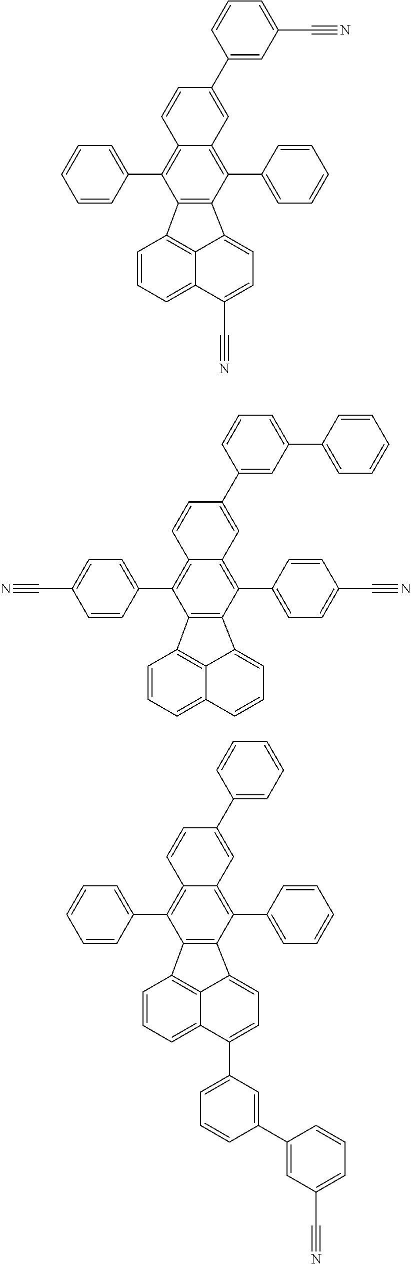

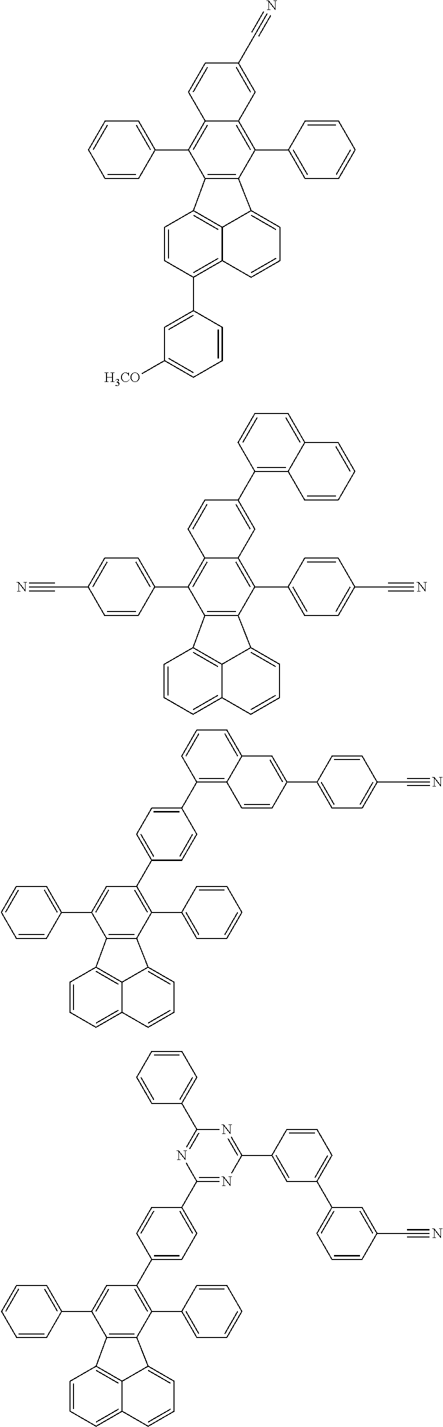

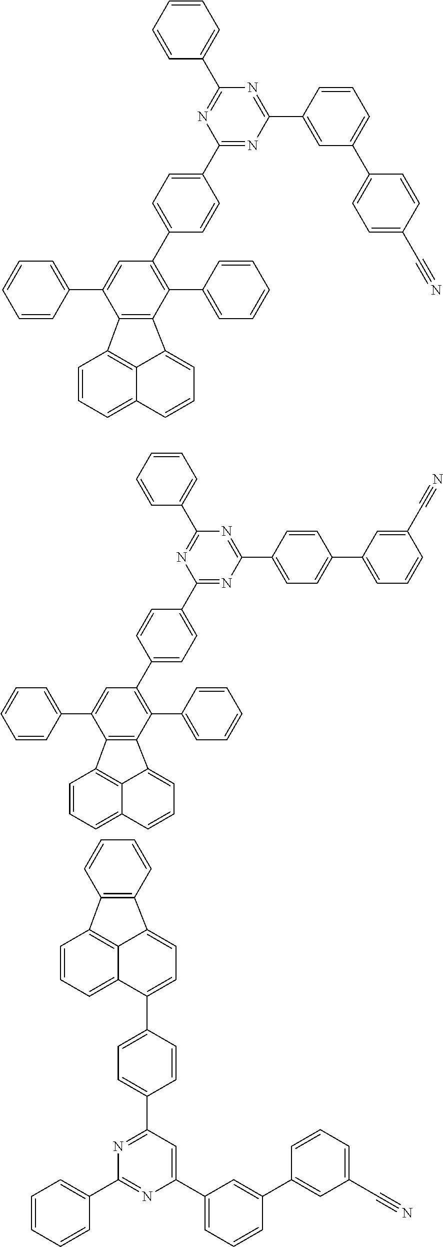

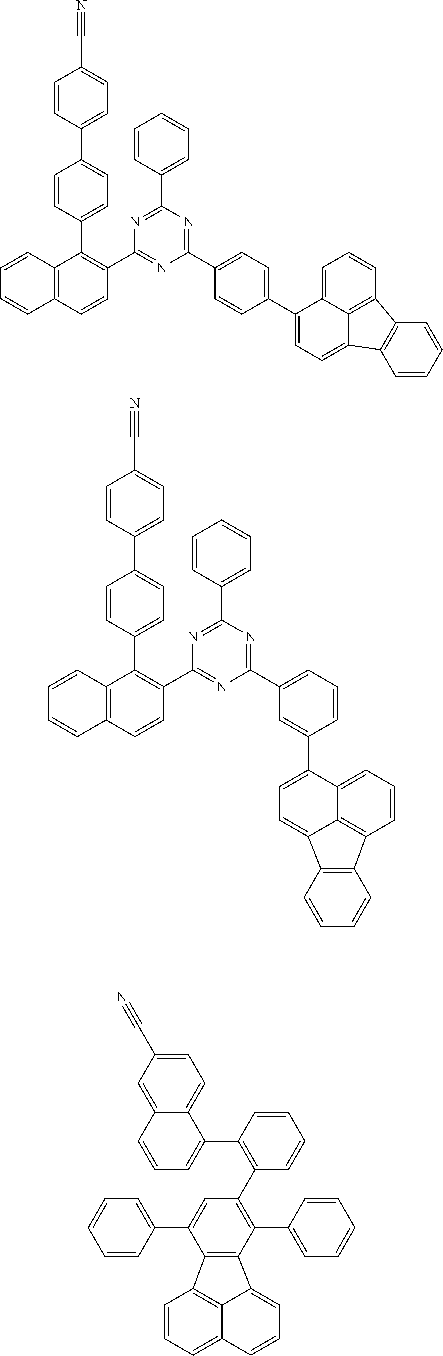 [SMILES: N#Cc1ccc(-c2ccc(-c3c(-c4nc(-c5ccccc5)nc(-c5ccc(-c6ccc7c8c(cccc68)-c6ccccc6-7)cc5)n4)ccc4ccccc34)cc2)cc1 ; N#Cc1ccc(-c2ccc(-c3c(-c4nc(-c5ccccc5)nc(-c5cccc(-c6ccc7c8c(cccc68)-c6ccccc6-7)c5)n4)ccc4ccccc34)cc2)cc1 ; N#Cc1ccc2c(-c3ccccc3-c3cc(-c4ccccc4)c4c(c3-c3ccccc3)-c3cccc5cccc-4c35)cccc2c1]

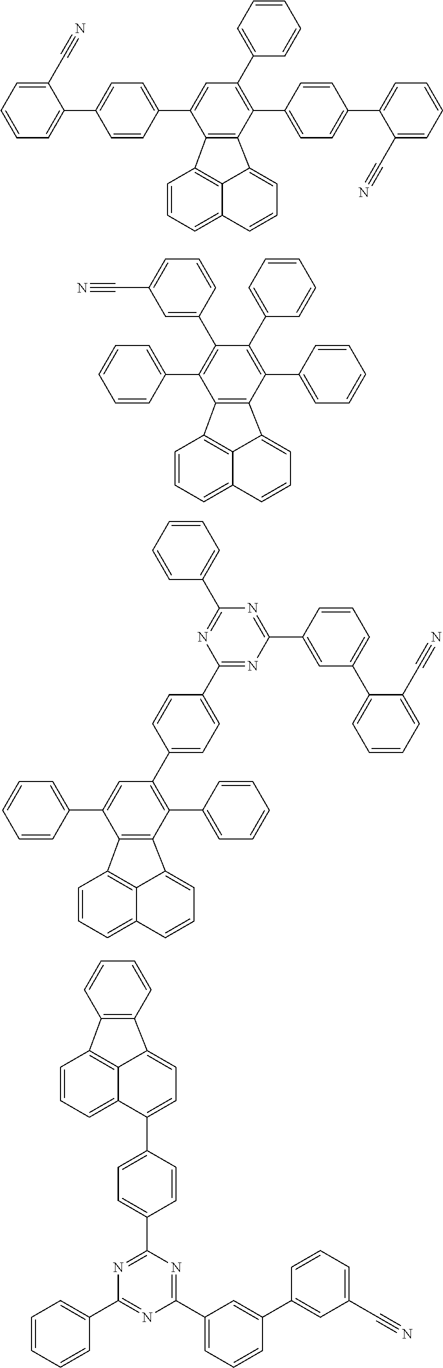

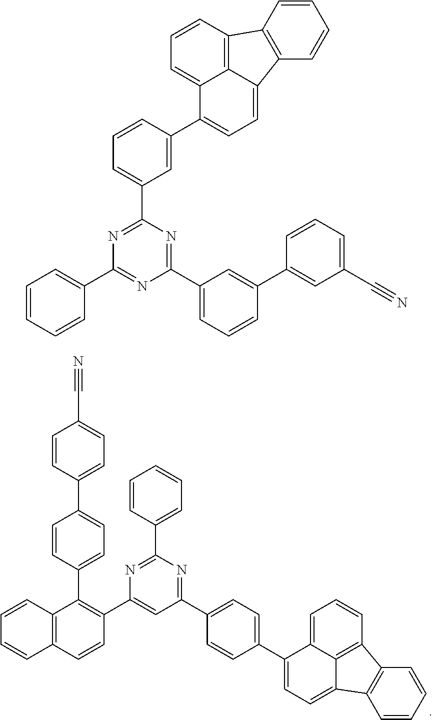

6. An organic light emitting device comprising:
a first electrode;
a second electrode provided opposite to the first electrode; and
one or more organic material layers provided between the first electrode and the second electrode,
wherein one or more layers of the organic material layers include the compound of claim 1.

7. The organic light emitting device of claim 6, wherein the organic material layer includes an electron injection layer, an electron transfer layer, or an electron injection and transfer layer, and the electron injection layer, the electron transfer layer, or the electron injection and transfer layer includes the compound.

8. The organic light emitting device of claim 6, wherein the organic material layer includes a hole blocking layer, and the hole blocking layer includes the compound.

9. An organic light emitting device comprising:
a first electrode;
a second electrode provided opposite to the first electrode; and
one or more organic material layers provided between the first electrode and the second electrode,
wherein one or more layers of the organic material layers include the compound of claim 5.
